(19) United States
(12) Patent Application Publication
(10) Pub. No.: US 2021/0046183 A1
(43) Pub. Date: Feb. 18, 2021

(54) COMPOSITIONS AND METHODS FOR PREVENTION AND TREATMENT OF IMMUNE COMPLEX DISEASE

(71) Applicant: Fred M. COWAN, Colora, MD (US)

(72) Inventor: Fred M. COWAN, Colora, MD (US)

(21) Appl. No.: 17/064,669

(22) Filed: Oct. 7, 2020

Related U.S. Application Data
(63) Continuation-in-part of application No. PCT/US2019/026712, filed on Apr. 10, 2019.
(60) Provisional application No. 62/656,084, filed on Apr. 11, 2018.

(51) Int. Cl. A61K 39/395; A61K 39/215; A61K 38/17; A61K 38/16

(52) U.S. Cl. CPC A61K 39/39566; A61K 39/215; A61K 2039/627; A61K 38/1774; A61K 38/164; A61K 38/1725

(57) ABSTRACT

The disclosure pertains to methods of immunotherapy for treating diseases and disorders which involve cellular Fc receptor mediated immune responses in humans and animals.

This application is a bypass continuation in part application of PCT/US2019/026712 filed Apr. 10, 2019, which claims benefit of U.S. Ser. No. 62/656,084 filed Apr. 11, 2018 the entirety of which are incorporated herein by reference.

### SPECIFICATION

### Background of the Invention

The disclosure pertains to methods of immunotherapy for treating diseases and disorders which involve cellular Fc receptor mediated immune responses in humans and animals. Abnormal or undesirable cellular Fc receptor mediated immune reactions are beneficially altered by locally or systemically administering, for example, an antibody binding molecule such as an immunoglobulin binding factor (“IBF”) reagent, for example, an Fc receptor polypeptide, Fc reagent, and a Fab antibody receptor, Fab reagent, in particular a multivalent protein having in its molecule several Fc receptor sites and/or a Fab receptors such as Staphylococcus protein A, a streptococcal Protein G, a streptococcal Protein G with the albumin binding site removed, or monovalent Fc receptor possessing only a single Fc receptor site and/or a Fab receptor. The disclosure further provides that the IBF reagent is further coupled and/or fixed to an antigen or antigen-antibody immune complex (“IC”) in a manner to act as an adjuvant-vaccine-checkpoint to enhance or inhibit immunity to said antigen or antigen antibody immune complex. Staphylococcus aureus protein A (“SPA”) has both Fc and Fab IBF, Fc reagent or Fab reagent actions. The mechanism of Fc receptor binding is constant for Fc antibody region and binding of Fab constant region of antibody to Fab IBF, thus and the resultant FcR immunity remains constant for all antigens as does the consistent Fc reagent and Fab reagent binding AND IBF action.

The immune system (“IS”) is vital to maintaining the health and physical integrity of the body. Primary functions of the immune system include detecting and destroying invading microbes and abnormal (e.g., cancer) cells, and detecting and promoting the healing of damaged tissues. Another primary function is to identify “self” tissues and suppress pathological autoimmune responses directed against self.

The immune system is divided into two branches, innate and adaptive immunity. Innate defenses against pathogen infection include the physical barriers formed by epithelia that cover the body's surfaces, and molecular receptors that recognize general features of pathogens. Such receptors activate complement proteins to attack pathogen membranes, and they stimulate phagocytes such as macrophages to engulf and destroy the pathogens. Activated complement and phagocytes also trigger inflammatory responses that promote the movement of antibodies and cytotoxic pathogen-killing cells of the adaptive branch, together with increased amounts of complement and phagocytes, to the site of infection.

Adaptive immune responses are driven by specific recognition of antigenic molecules by receptors on the surfaces of T cells and B cells, and by soluble antibodies that bind with high specificity and affinity to antigens to form immune complexes. Non-self, or “foreign,” antigens that are bound by lymphocyte receptors and antibodies include antigens of microbes, damaged tissue, and cancer neoantigens. Lymphocytes sometimes mistakenly recognize self antigens, which can result in autoimmune disease. Immune mechanisms are much the same for all antigens. The binding of antigen-specific lymphocyte receptors and antibodies to epitopes or determinants of “foreign” antigens on pathogens and cancer cells induces protective responses by diverse immune cell populations. These culminate in destruction of the pathogenic cells by cytotoxic T cells (cellular immunity), and in antibody-mediated elimination of antigenic molecules, pathogens, and abnormal cells (humoral immunity). A key feature of adaptive immune responses is the ability of lymphocytes to develop long lasting memory of specific antigens, such that protective immune responses are generated significantly faster after re-infection than they were after the initial infection. Long-term memory and specific recognition of pathological antigens by lymphocytes and antibodies enable the protective immunity produced by vaccination.

The immune system maintains homeostasis in the health of the body through wound healing and defense against microbial pathogens and cancer. In turn, the immune system itself is homeostatically governed by complex networks of regulatory signals that coordinate activities of the innate and adaptive branches, and maintains a balance between the induction of “positive,” stimulatory immune responses, and “negative,” suppressive responses. Positive, stimulating regulatory reactions activate pathogen destroying and wound healing immune responses following infection or injury. Negative regulatory signaling downregulates defensive immune responses; for example, in returning to the normal resting state after actively responding to infection or injury. Negative signaling can also suppress immune responses and induce tolerance towards self antigens that are recognized by lymphocyte receptors, thereby preventing pathological autoimmune responses. (Guy A. Viosin, “Immunity and Tolerance: A Unified Concept,” Cellular Immunology 2:670-689, 1971)

In human physiology, the complexity of the immune system and its regulation is superseded only by that of the brain. Further the nervous and immune systems interact with many molecules having dual function in both systems a basis for the field of Neuroimmunology and collateral linkage of homeostasis and disease to both systems. Notwithstanding, regulation of the immune system is largely mediated by structure-specific binding interactions between soluble and cell-bound receptors and their ligands that promote either activation or inhibition of immune responses. The importance of such “lock and key” binding interactions in immune regulation was recognized by Paul Ehrlich over a century ago. (C. Prull, “Part of a Scientific Master Plan” Paul Ehrlich and the Origins of his Receptor Concept,” 2003, Medical History 47: 332-356). The control and feedback systems that regulate the balance of positive and negative immune responses are mediated by biochemical signaling pathways formed of binding interactions between cell-bound and soluble receptors and ligands. Soluble extracellular signaling molecules that coordinate and regulate immune responses include (but are not limited to) antibodies, antigens, immune complexes, cytokines, chemokines, and autacoids and even neurotransmitters or dual function immune molecules and neurotransmitters (Neuroimmunology). There are many different types of receptors, cell-bound or soluble, inside or outside of cells of the immune system, that, upon binding to their ligands, initiate signaling to stimulate positive, protective immune responses, e.g. to destroy pathogens or cancer cells, or repair tissue damage. Likewise, there are many different receptors that, upon binding their ligands, generate negative, inhibitory signals that induce suppression of innate and adaptive immune responses; e.g., to govern an active anti-pathogen response, or to prevent active, autoimmune responses against self tissues.

Upon binding to their ligand, some regulatory receptors may initiate either positive signaling to activate effector responses, or negative signaling to inhibit responses, depending on signals they receive from other regulatory molecules. For example, activation of a naïve T cell requires signaling by two independent receptor-ligand interactions. The T cell receptor must bind specifically to a foreign antigen presented on the surface of an antigen presenting cell (APC), and a second receptor protein on the T cell surface, called “CD28,” must also bind to another protein ligand on the surface of the APC, called “B7.” If the T cell receptor binds specifically to the foreign antigen presented by the APC and the CD28 receptor also binds to the B7 ligand, the T cell is activated and undergoes clonal expansion and differentiation. However, if the T cell receptor binds to the foreign antigen on the APC, but the CD28 receptor is unable to binds to the B7 ligand, T cell responses are inhibited and the T cell becomes anergic, tolerant of the antigen. Because their binding to one another sends a costimulatory signal that is required to activate T cells to respond to foreign antigens, the CD28 receptors on T cells and the B7 ligands on APCs are called “costimulatory” receptors and ligands, respectively.

Activation of a T cell induces increased expression of the ligand receptor CTLA-4, which acts as a brake to restrain T cell effector activities. CTLA-4 binds specifically to the B7 ligand with much greater affinity than the CD28 receptor, so it blocks the binding of CD28 to B7 and inhibits CD28-dependent T cell activation. Because the binding of CTLA-4 to B7 blocks costimulation by CD28 and sends a negative signal that inhibits T cell activation, CTLA-4 receptors are called “coinhibitory” receptors, and their effects on positive, T cell activation are called “coinhibition.” In 1999 Sinclair identified about 30 coinhibitory receptors on lymphocytes and other cells of the reticuloendothelial system in addition to CTLA-4, and stated that “others are being defined regularly.” (N. R. StC. Sinclair, “Why so many coinhibitory receptors?,” Scandinavian Journal of Immunology, 50(1): 10-3 Jul. 1999)

Fc-specific Abs and microbial and viral Fc-binding and Fab-binding proteins interact with and modulate this regulatory network, e.g., causing pathological responses such as rheumatoid arthritis, or to evade defensive host immune responses.

To avoid detection and destruction by the immune system, bacteria and other pathogens have evolved multiple molecular systems that disrupt/inhibit/modulate the systems that regulate the homeostatic balance between immune activation/rejection & tolerance/suppression.

Immune inhibitory checkpoints are receptor-ligand mediated signaling pathways that suppress immune responses, like braking a car, and protect against autoimmunity. The 2018 Nobel Prize in Physiology or Medicine was awarded jointly to James P. Allison and Tasuku Honjo for showing that inhibiting immune checkpoints CTLA-4 and PD-1, respectively, can release the brake and unleash immunity against cancer. This work overcame dogma that immunotherapy in animals is not relevant to humans, and revitalized the field of immunotherapy. (www.nobelprize.org/prizes/medicine/2018/press-release/).

In 2018 the prestigious Robert Koch Award was given to Dr. Jeffery Ravetch for his “groundbreaking work” cloning and studying the different types of Fc receptors (FcR) on the surfaces of immune cells, and how the FcR interact with antibodies and immune complexes (IC) to regulate immune responses through “a balance of both activating and inhibiting signals.” www.rockefeller.edu/news/22523-jeffrey-v-ravetch-to-receive-2018-robert-koch-award/

The discoveries recognized by these different awards are connected, as evidence indicates that anti-tumor activities induced by monoclonal antibodies targeting CTLA-4 and the PD-1 ligand PD-L1 are modulated by FcR interactions. (Simpson T R, James P. Allison J P. Fc Gamma R. #Springer Science+Business Media New York 2017 J. L. Marshall (ed.), Cancer Therapeutic Targets, DOI 10.1007/978-1-4419-0717-2_140. and Chen X, Song X, Li K, Zhang T. FcγR-Binding Is an Important Functional Attribute for Immune Checkpoint Antibodies in Cancer Immunotherapy. Front Immunol. 2019 Feb. 26; 10:292).

### FcR are Immune Checkpoints

Analogous to the immune checkpoints described by Drs. Allison and Honjo, the binding of IC to FcR has long been recognized as a nexus of homeostatic immune regulation, governing both activation and suppression of immune responses, and an important target for “immune checkpoint” drugs and FcR IBF treatment of IC disease.
“Much effort has been expended to activate the immune response by employing immune stimulators. The fact that the immune system must contain its own set of checks and balances to avoid over reaction has received less attention. The neutralization of immune complex inhibition of antibody-dependent cellular toxicity demonstrates how at least one form of cellular immunity can be regulated by relative concentrations of sera factors and lymphocyte receptors. If the binding of antigen-antibody complexes to IgG Fc cellular receptors is a major mechanism of immune regulation, Staphylococcus aureus protein A could prove a powerful tool for studying immune mechanisms and perhaps altering immune response.”
   (Cowan F M, Klein, D L, Armstrong, G R, Pearson, J W. (1979). Neutralization of immune complex inhibition of antibody dependent cellular cytotoxicity in vitro by Staphylococcus aureus protein A. Biomedicine, 30, 23-27). These perceptions, based on the work of many researchers, have proved true for Fc receptors, T cell immunity, cancer immunotherapy, autoimmune disorders, and many other immune pathologies (reviewed, U.S. Pat. No. 5,189,014, Feb. 23, 1993 (original application filled Dec. 3, 1979; reviewed, Cowan F M, Klein D L., Armstrong G R, Stylo, W A and Pearson J W. (1980) Fc receptor mediated immune regulation and gene expression. Biomedicine, 32, 108-110; Cowan F M. Connecting science past and future. J Chin Med Assoc 2017; 80:1-2).

Before the term “immune checkpoints” became widely used, Dr. Nicholas R. StC. Sinclair applied the term “coinhibitory receptors” to receptors such as CTLA-4, PD-1, and inhibitory FcR (FcγRIIB), describing them as transmembrane glycoprotein receptors “that display negative signaling functions on lymphocytes and other cells of the reticuloendothelial system” and “are armed by the action of activating receptors and inhibit signaling by activating receptors.” (Sinclair N. R. StC. “Why so many coinhibitory receptors?,” Scandinavian Journal of Immunology, 50(1): 10-3 Jul. 1999). Tiege et al. describe Fc gamma receptors (FcgRs) as, “therapeutically important immune checkpoints.” and propose referring to the activating and inhibitory FcγRs as activating and inhibitory “antibody checkpoints,” respectively. (Teige I, Mirtensson L, Frendéus B L. Targeting the Antibody Checkpoints to Enhance Cancer Immunotherapy-Focus on Fc RIIB. Frontiers in Immunology|www.frontiersin.org 1 Mar. 2019|Volume 10|Article 481). For the purpose of describing the invention, the term “FcR antigen specific checkpoint” is used herein to refer to both positive (activating) and negative (inhibitory) FcR of all types that regulate immune effector cell activity or modulate immune responses.

### Relevant Events in the History of Immunotherapy

The history of cancer immunotherapy reaches back to c 2600 BC, when the Egyptian physician Imhotep recommended treating tumors (swellings) with a poultice followed by incision, which would inevitably lead to an infection at the tumour site. In the 1890s in New York, Dr. William Coley saw a patient's “incurable” facial sarcoma go into go into complete remission after the tumor site became severely infected with Streptococcus pyogenes and the patient developed a high fever. Suspecting the microbial infection and fever induced the cure, Dr. Coley eventually developed a cancer vaccine containing two killed bacteria, Streptococcus pyogenes and Serratia marcescens, that became known as “Coley's toxins”. Hundreds of patients with sarcomas as well as carcinomas, lymphomas, melanomas, and myelomas were treated with Coley's toxins by Dr. Coley and his contemporaries, and many complete remissions and cures resulted, even in patients in their final stages of disease. (Hoption Cann S A, van Netten J P, van Netten C. Dr William Coley and tumour regression: a place in history or in the future. Postgraduate Medical Journal 2003; 79:672-680.)

In 1910, Austrian physicians Ernest Freund and Gisa Kaminer noticed that blood sera of healthy people could dissolve cancer cells whereas sera of cancer patients did not, and they proposed that sera of cancer patients contained “anti-carcinolytic” factors that protected cancer cells from a destructive agent in normal serum.

In 1959 Dr. Lloyd Old showed that iv injection of BCG (attenuated animal tubercle bacilli) into mice prior to tumor challenge slowed tumor growth and prolonged survival, and BCG is now widely used to treat superficial bladder cancer. He was also a leader in the search for tumor-specific antigens that may serve as cancer biomarkers and targets for vaccines and other immunotherapies. (www.cancerresearch.org/news/2011/cri-director-lloyd-old-dies-of-prostate-cancer).

In the 1960 and '70s in Seattle, Drs. Karl and Ingegerd Hellström observed that immune complexes (IC) in serum of mice with chemically induced tumors can act as “blocking factors” that block immune reactions against the tumors. (Sjögren H O, Hellström I, Bansal S C, Hellström K E. Suggestive Evidence That the “Blocking Antibodies” of Tumor-Bearing Individuals May Be Antigen-Antibody Complexes. Proc Natl Acad Sci USA. 1971 June; 68(6):1372-5; Moss, R W, www.mossreports.com/history-of-immune-checkpoint-inhibition/; and www.whatisbiotechnology.org/index.php/science/summary/checkpoint-inhibitor/immune-checkpoint-inhibitors-are-key-cancer-treatment-tool). Such antibody-containing “blocking factors” can be removed from tumor-bearer sera by absorption to Staphylococcal protein A. (Steele G Jr, Ankerst J, Sjögren H O, Vang J, Lannerstad O. Absorption of blocking activity from human tumor-bearer sera by Staphylococcus aureus, Cowan I. Int J Cancer. 1975 Feb. 15; 15(2):180-9).

In 1966 Berken and Benacerraf showed that antibodies and ICs bind to macrophages through their Fc portion. (Berken A, Benacerraf B. Properties of antibodies cytophilic for macrophages. J Exp Med. 1966 Jan. 1; 123(1):119-44.)

In 1968 Sinclair et al. demonstrated that immune suppression by antibodies requires the Fc fragments. (Sinclair, N. R. StC., Lees, R. K., Elliott, E. V. (1968) Role of Fc fragment in the regulation of the primary immune response. Nature, 220, 1048 1049.)

In 1979 Fred M. Cowan, Jr. filed a U.S. patent application that describes methods of immunotherapy comprising administration of exogenous polypeptides having a cellular FcR-like binding capability for endogenous antibodies and ICs, to treat FcR-mediated immune disorders in humans and animals, including methods for treating cancer and autoimmune disorders. (Fred M. Cowan, Jr., U.S. Pat. No. 5,189,014, Feb. 23, 1993).

In the 1980s multiple groups reported that administration of Staphylococcal protein A (SPA), a microbial FcR IBF, can induce antitumor activity in animals. (Ray, P. K., Bandyopadhyay, S., Dohadwala, M. et al. Antitumor activity with nontoxic doses of protein A. Cancer Immunology, Immunotherapy, October 1984, Volume 18, Issue 1, pp 29-34; R. W. Engelman, R. D. Tyler, L. Q. Trang, W. T. Liu, R. A. Good, N. K. Day. Clinicopathologic responses in cats with feline leukemia virus-associated leukemia-lymphoma treated with staphylococcal protein A. Am J Pathol. 1985 March; 118(3): 367-378; Cowan F M, Klein D L, Armstrong G R, Ablashi D V, Pearson J W, Bensinger W I. Inhibition of rat adenocarcinoma metastases by “Staphylococcus aureus” protein A. Biomed Pharmacother. 1982 January; 36(1):29-31.) Immunotherapy has experienced a “dark age” of lack of understanding, interest and funding (For James Allison, PhD, Perseverance and Hard Science Are Paramount in Cancer Research By Ronald Piana May 25, 2018. ASCO Post. www.ascopost.com/issues/may-25-2018/james-allison-perseverance-and-hard-science-in-cancer-research/?email=82996f5e18210672858ab4259c70d6418c72a87d22b1dcbe274bc05fac84ee17 During the 1980s and 1990s scientific interest in cancer immunotherapy faded, until Drs. Allison and Honjo reawakened interest in the field by showing that immune checkpoint inhibition can activate immune responses against cancer in human patients. The lack of knowledge and citation of prior art on IC-FcR interaction has impaired progress and reflects not just an emphasis on more recent research and some disassociation between science and patent systems (Cowan F M. Connecting science past and future. J Chin Med Assoc 2017; 80:1-2; Concord and Conflicts Blur Science and Invention. Fred Cowan. Opinion, The Scientist, Mar. 30, 1998 www.the-scientist.com/?articles.view/articleNo/18875/title/Concord-And-Conflicts-Blur-Science-And-Invention/). This gap broke continuity in the record of discovery and hampered exchange of ideas between researchers. The many chasms separating collective knowledge combined to at times stall and continue to hinder transfer of research on immunity to clinical application; and such “systemic” fractures impacts not just science citation and intellectual property prior art but utility in patient care.

Virulence factors, e.g. the numerous & diverse VFs of S. aureus. Invading pathogens and tumor cells evade destruction by the immune system by producing molecules that interact with immune checkpoints to downregulate or block defensive immune responses.

Cancer co-opts immune checkpoints to induce tolerance and evade destruction by the immune system. Receptor-ligand interactions that regulate T cells are of central importance because they can activate either positive, defensive immune responses or negative, suppressive responses, depending on their interactions with co-stimulatory or co-inhibitory molecules.

The new immune checkpoint inhibitors (e.g. YERVOY® (ipilimumab) & KEYTRUDA® (pembrolizumab)) block the signaling to checkpoints to dampen immune responses, thereby allowing the immune system to be activated to recognize and attack the invading pathogens or tumor cells.

Receptor ligand regulated immunity can be both positive and negative therefore the following focus on positive immunity to cancer neoantigens is exemplary not limiting. The complexity of immunity has contributed to both a lack of understanding and appreciation of the role of immunity in cancer immunotherapy. The field is now focused on very important and commendable work on checkpoint drugs identified over the last two decades by Dr Allison and others (Suzanne L. Topalian, Charles G. Drake, Drew M. Pardoll. Immune Checkpoint Blockade: A Common Denominator Approach to Cancer Therapy. Cancer Cell 27, Apr. 13, 2015). However, most of these checkpoints have a disadvantage of being antigen nonspecific. Further these molecules are antibodies often containing an active Fc region that could collaterally influence Fc receptor immunity (Mark J Smyth, Shin Foong Ngiow, Michele W L Teng. Using FcR to suppress cancer. Targeting regulatory T cells in tumor immunotherapy. Immunology and Cell Biology (2014) 92, 473-474.) Even microbial molecule IC not specific to cancer can up regulate immunity to cancer (Anissa S. H. Chan, Adria Bykowski Jonas, Xiaohong Qiu, Nadine R. Ottoson, Richard M. Walsh, Keith B Gorden, Ben Harrison, Peter J. Maimonis, Steven M. Leonardo, Kathleen E. Ertelt, Michael E. Danielson, Kyle S. Michel, Mariana Nelson, Jeremy R. Graff, Myra L. Patchen, Nandita Bose* Imprime PGG-Mediated Anti-Cancer Immune Activation Requires Immune Complex Formation. PLOS ONE|DOI:10.1371/journal.pone.0165909 Nov. 3, 2016).

Checkpoint drugs are often comprised of monoclonal antibody that contain Fc constant region that can bind to FcR. Many of the actions of such checkpoint drugs are now known to be FcR mediated and often independent of their checkpoint actions. FcR mediated checkpoint drug actions is predicted to be due to only ADCC checkpoint activation PD-1, CTL-4 and phagocytosis inhibition of some checkpoints that actually significantly bind their target molecule, PD-L1 (Xuexiang Du, Fei Tang, Mingyue Liu, Juanjuan Su, Yan Zhang, Wei Wu, Martin Devenport, Christopher A Lazarski, Peng Zhang, Xu Wang, Peiying Ye, Changyu Wang, Eugene Hwang, Tinghui Zhu, Ting Xu, Pan Zheng, Yang Liu. A reappraisal of CTLA-4 checkpoint blockade in cancer immunotherapy. Cell Research (2018) 0:1-17; doi.org/10.1038/s41422-018-0011-0), (Rony Dahan, Emanuela Sega, John Engelhardt, Mark Selby, Alan J. Korman, Jeffrey V. Ravetch, FcgRs Modulate the Anti-tumor Activity of Antibodies Targeting the PD-1/PD-L1 Axis. Cancer Cell 28, 285-295, Sep. 14, 2015). Finally, many cellular receptors to include CTLA4 (Saverino D, Simone R, Bagnasco M, Pesce G. The soluble CTLA-4 receptor and its role in autoimmune diseases: an update. Auto Immun Highlights. 2010 Nov. 4; 1(2):73-81) have a soluble form like FcR IBF or TNF receptors that are proposed as or have been used as “receptor drugs.” CTLA4 monoclonal binds soluble CTLA4s forms IC and activates FcR immunity, similar to the antineoplastic action of Imprime IC and CTLA4s would perhaps make for a good receptor drug, like proposed for FcR IBF pioneer patent.

FcR-IC interaction influences T cell mediated immunity, “Even though earlier studies documented the presence of low-affinity FcRs on CD4+ T-cells, neglect in examining the contribution of these receptors in CD4+ T-cell responses over the past two decades has hampered progress in establishing the contribution of FcRs to adaptive immune responses. Emerging data reconfirm some of the earlier findings that activated CD4+ T-cells not only express FcRs, but signaling via these receptors modulates adaptive immune responses. Engagement of FcRs by the ligand contributes to the development of CD4+ effector T-cell responses. Low-affinity FcRs are critical for innate immune responses, and their presence on CD4+ T-cells, cells of adaptive immunity, suggests their critical role in adaptive immunity.” (Anil K. Chauhan. Human CD4+ T-Cells: A Role for Low-Affinity Fc Receptors. Frontiers in Immunology|www.frontiersin.org June 2016, Volume 7, Article 215), (Cowan, F. M. A Method for Treating Cellular Fc Receptor Mediated Immune Disorders. U.S. Pat. No. 5,189,014, Feb. 23, 1993 (original application filed 1979). While the authors of FcR mediated checkpoint drug actions point to mechanisms such as ADCC and phagocytosis the Fc constant region of checkpoint drugs could engage the full scope of FcR mediated immunity; to include checkpoints binding their soluble ligands and form IC or cellular ligands that further crosslinking with FcR on T cells or other cells to regulate immunity and have efficacy for cancer. Imprime's yeast IC antigen nonspecific activation of cancer immunity may also have implication for checkpoint drug IC. (Anissa S. H. Chan, Adria Bykowski Jonas, Xiaohong Qiu, Nadine R. Ottoson, Richard M. Walsh, Keith B Gorden, Ben Harrison, Peter J. Maimonis, Steven M. Leonardo, Kathleen E. Ertelt, Michael E. Danielson, Kyle S. Michel, Mariana Nelson, Jeremy R. Graff, Myra L. Patchen, Nandita Bose* Imprime PGG-Mediated Anti-Cancer Immune Activation Requires Immune Complex Formation. PLOS ONE|DOI:10.1371/journal.pone.0165909 Nov. 3, 2016.) FcR positive or negative regulatory signals of effector functions may be critical to the efficacy of checkpoint drugs. IBF bond to cancer IC or checkpoint IC or other IC would greatly augment FcR-IC mediated immunity and efficacy. It is a particular advantage of IBF-checkpoint antibody drugs that the IBF-checkpoint antibody drug can engage its ligand/receptor after in vivo injection to form the IBF-checkpoint-IC that promotes FcR immunity and efficacy. Further linker such as biotin-avidin conjugates can be used to promote such IBF-checkpoint antibody-IC in vitro or in vivo.

Immunotherapy has a rich history spanning five millenniums, with strong science technology foundations of well over a century, and intensive effort and much better understanding over the last half century. This has more recently predicted and produced discovery of in addition to Fc Receptor (“FCR”) other checkpoint regulators and “checkpoint blockers” that confirmed the merit of earlier concepts. So far immunotherapy for major cancers lacks precision leaving an average complete response to major metastatic cancer of about 7% the same as for each element of the triad of surgery, chemotherapy and radiation (Brendon J Coventry, Martin L Ashdown. Complete clinical responses to cancer therapy caused by multiple divergent approaches: a repeating theme lost in translation. Cancer Management and Research 2012:4 137-149). The potential for predictive biomarker for efficacy and response, synergy between the many checkpoint regulators, other immunotherapy and peripherally the anticancer and immune modulating efficacy of the triad and anti-inflammatory compounds or drugs could improve efficacy. Immunotherapy using a synergy of multiple agents is yielding some better results but also increased toxicity. Checkpoint drugs can also cause hyperprogression of cancers that can also occur with chemotherapy that quickly produce terminal disease (Campiat S, Dercle L, Ammari S, et al. Hyperprogressive disease (HPD) is a new pattern of progression in cancer patients treated by anti-PD-1/PD-L1. Clin Cancer Res. 2016; 23(8):1920-1928. doi: 10.1158/1078-0432.ccr-16-1741). The Clinicians that observed hyperprogression did not at first report it because they did not understand the history of immune enhancement of cancer (Viosin G A. Immunity and Tolerance a Unified Concept. Cellular Immunology 1971, 2, 670-89).

The cancer remissions caused by bacteria infection that contain immunoglobulin-binding factor (IBF) activity has an intellectual bloodline from Imhotep to Coley's toxins to the pioneer patent of Cowan on Fc receptor IBF drugs. The concept of cancer being the result of anomalous tolerance to cancer antigens was proposed and reviewed by Voisin nearly half a century ago. In that same time-frame, the original work on “blocking factors” by the Hellstroms (H. O. Sjögren, I. Hellström, S. C. Bansal, and K. E. Hellström. Suggestive Evidence That the “Blocking Antibodies” of Tumor-Bearing Individuals May Be Antigen-Antibody Complexes. Proc. Nat. Acad. Sci. USA Vol. 68, 6, 1372-1375, June 1971; (Ralph Moss. HISTORY OF IMMUNE CHECKPOINT INHIBITION. /www.ralphmossblog.com/2017/02/history-of-immune-checkpoint-inhibition.html) demonstrated antigen antibody complex feedback could down regulate lymphocyte immunity to cancer. Such cancer antigen antibody immune complex blockade of immunity was antigen specific, FcR mediated and could be reversed by FcR-like IBF (Cowan, F. M. A Method for Treating Cellular Fc Receptor Mediated Immune Disorders. U.S. Pat. No. 5,189,014, Feb. 23, 1993 (original application filled 1979) (Cowan, F. M., Klein, D. L., Armstrong, G. R., Stylos, W. A. and Pearson, J. W. (1980). (Fc receptor mediated immune regulation and gene expression. Biomedicine, 32, 108-110).

IBF immunotherapy yielded antigen specific checkpoint-like reversal of immune complex inhibition of lymphocyte immunity to the cancer antigen. IBF immunotherapy antigen specificity reduces the likelihood of autoimmune or inflammatory side effects seen with standard checkpoint drugs and can also be used to generate cancer antigen specific immunity that can be further amplified by synergy with antigen nonspecific immunotherapy such as checkpoint drugs. Microbial IBF are preferred because they have over millions of years of evolution evolved to alter host immunity in ways that can be usurped for immunotherapy. Further biomarkers of inflammation that can impair cancer immunity such as complement reactive protein (CRP), cytokines and immune complex can be assayed to both identify potential responders to immunotherapy and degree of response to said therapy. Finally, drugs or compounds that have demonstrated anti-inflammatory and anti-cancer pharmacology and/or influence FcR mediated response such as statins, heparin, metformin or turmeric/curcumin can reduce the inhibitory immunity boarding the immunotherapy responders and increasing response. (Karen S. Anderson, Joshua LaBaer. The sentinel within: exploiting the immune system for cancer biomarkers. Proteome Res. 2005; 4(4): 1123-1133), (Cowan, F M, Broomfield, C A, Stojiljkovic, M P, Smith, W J. A Review of Multi-Threat Countermeasures against Chemical Warfare and Terrorism. Military Medicine, 169, 11:850, 2004.)

With regard to the basis for immunotherapy, specific receptor-ligand interactions are also central to inducing anti-tumor immune responses in new methods of cancer immunotherapy.

The root problem discussed for decades is that with immunotherapy, for example, towards altered self neoantigen for treatment of, for example, cancer, it is difficult to achieve a balance between rejection and tolerance of the immunotherapy. Cancers with more mutated genes have the greater number of mutated antigens for immune identification and have better prognosis for immunotherapy. Most checkpoint regulators are antigen nonspecific and just increase the body's existing immunity to antigen. This can be beneficial or sometimes pathological causing pathological inflammation, autoimmunity or hyper-progression of cancer. If the immune response you are enhancing is too weak or misdirected you get no efficacy or perhaps autoimmunity. There are other forms of immunotherapy such as vaccination to cancer antigens that specifically increase immunity to cancer-self antigens.

The use of microbial FcR mimetics such as Staphylococcus Protein A (SPA), a major secretory product of staphylococcal infections, or streptococcal Protein G, or streptococcal Protein G with the albumin binding site deleted, in therapeutic methods associated with tumor remission relates the ancient observations of Imhotep and Coley's discoveries of bacterial toxin immunotherapy to the successes of modern molecular immunotherapy.

Fc receptors (FcR) are known to be a focal immune regulatory molecule for both innate, adaptive antibody and cellular immunity and coordination of all these elements. The diversity of FcR actions as a cellular receptor and soluble receptor places it under many immunological lexicons to include cytokine and checkpoint. The defining function of FcR binding to antibody Fc constant regions allowing these receptors to specifically influence immunity to any antigen bound to the Fab variable region of antibody. (reviewed, (Cowan, F. M., Klein, D. L., Armstrong, G. R., Stylos, W. A. and Pearson, J. W. 1980. Fc receptor mediated immune regulation and gene expression. Biomedicine, 32, 108-110), (Sinclair N R StC. Immunoregulation by Antibody and Antigen-Antibody Complexes. Proc. 1978, 10, 349-53), Sinclair N R StC. & Panoskaltsis A. Immunoregulation by Fc signals A mechanism for self-nonself discrimination. Immunol Today. 1987; 8(3):76-9), (Hogarth P M, Pietersz G A. Fc receptor-targeted therapies for the treatment of inflammation, cancer and beyond. Nat Rev Drug Discov. 2012; 11:311e31), N. R. StC. Sinclair, “Why so many coinhibitory receptors?,” Scandinavian Journal of Immunology, 50(1): 10-3 Jul. 1999).

Antigen antibody immune complex (IC) further engaging FcR is a cornerstone of positive and negative immune regulation to include antigen specific immunity. Immune complexes (“IC”) are present in the sera of cancer patients and can sometimes act as blocking factors to FcR mediated checkpoints inhibiting cellular immunity to cancer neoantigens (Hellstroms).

Many cellular receptors do not just reside on cells but circulate in soluble humoral form, such soluble FcR are immunoglobulin binding factors (IBF) that moderate IC-FcR binding, FcR mediated immunity and immune regulation (Léonetti M, Galon J, Thai R, Sautès-Fridman C, Moine G, Menez A. Presentation of antigen in immune complexes is boosted by soluble bacterial immunoglobulin binding proteins. J Exp Med. 1999 Apr. 19; 189(8):1217-28).

Microbial FcR mimics can act as IBF to influence mammalian immunity to allow the bacteria to evade immune response, and such microbial IBF activity can be usurped for immunotherapy particularly cancer immunotherapy (Cowan, F. M. A Method for Treating Cellular Fc Receptor Mediated Immune Disorders. U.S. Pat. No. 5,189,014, Feb. 23, 1993 (original application filled 1979).

Further microbial IBF have many associated actions to include superantigen, and bind other mammalian molecule besides FcR (list) to include the Fab constant region of antibody. This gives microbial IBF the benefit of millions of years to evolutionary interaction to alter host immunity or function as immunotherapy.

The disclosure combines elements of, for example, IBF adjuvant, FcR checkpoint regulation, and IC vaccination to achieve better efficacy and less toxicity for treatment. The disclosure adds both safety and precision, to include an antigen specific “checkpoint” that can act as an adjuvant for synergy with other nonspecific check point regulators/blockers. Further immune “biomarkers”, for example, complement reactive protein (“CRP”) can identify sub populations of patients that will respond to immunotherapy and possible predict ways to make non-responders respond. Further, IC are also central biomarkers of immunity that can predict efficacy of immunotherapy. Certain drugs such as low molecular weight heparin (LMW heparin), statins, metformin and compounds such as turmeric/curcumin have efficacy for cancer. These drugs can reduce CRP and underlying inflammation and some directly influence IC-FcR mediated immunity and may augment immunotherapy (S. J. Drakeley, P. N. Furness. Heparin inhibits the binding of immune complexes to cultured rat mesangial cells. Nephrol Dial Transplant (1994) 9: 1084-1089), (J. D. Loike, D. Y. Shabtai, R. Neuhut, S. Malitzky, E. Lu, J. Husemann, I. J. Goldberg, S. C. Silverstein. Statin Inhibition of Fc Receptor-Mediated Phagocytosis by Macrophages Is Modulated by Cell Activation and Cholesterol. Arterioscler Thromb Vasc Biol. 2004, 2051-56.)

Inflammation can enhance cancer and pathological mutations, metastasis, growth and evasiveness (Orsolya Kiraly, Guanyu Gong, Werner Olipitz, Sureshkumar Muthupalani, Bevin P. Engelward. Inflammation-Induced Cell Proliferation Potentiates DNA Damage-Induced Mutations In Vivo. PLOS Genetics|DOI:10.1371/journal.pgen.1004901 Feb. 3, 2015 1/24). Statin use increases cancer survival for the conventional triad of cancer therapy. Biomarkers that reduce treatment populations to responder patients can increase efficacy in this subpopulation.

The disclosure combines, for example, IBF adjuvant, IC antigen specific vaccination, and FcR mediated checkpoint regulation. This can further be enhanced by predictive immune biomarkers and synergy of drugs and compounds and other immunotherapy that further measure or influence IC-FcR mediated immunity and associated inflammation.

The current disclosure also envisions the binding of microbial IBF, containing both Fab and FcR reagent actions, bound to IC to achieve immunotherapy. It is to be noted that in such a molecule, either IC or IBF can be isolated from sera standard isolation and/or culture procedures, or modified by known means to produce “equivalent” immunotherapy constructs with improve valance, number of binding sites, affinity of binding, and can further be made by or synthesized by amino acid sequence and synthesis. Such IBF-IC and equivalent immunotherapy constructs is especially suited for, for example, cancer and will not disassociate in immunoglobulin excess in patients' sera.

The current disclosure envisions the binding of microbial IBF containing both Fab and FcR reagent actions bound to IC to achieve immunotherapy. It is to be noted that such molecule, either IC of IBF can be isolated from sera standard culture procedures, or modified by known means to improve valance, number of binding sites, affinity of binding, and can further be made by or synthesized by amino acid sequence and synthesis. Such IBF-IC immunotherapy is especially suited for cancer and will not disassociate in immunoglobulin excess in patients sera.

It is an advantage of aspects of the disclosurethat the IBF-IC vaccine can be administered by systemic injection, incubated with blood, sera, and/or selected cell populations such as T cells. Further, the cells can be administer to the patient after incubation or further expanded in cell culture before infusion to the patient. It is a particular peripherally advantage that free IBF might be injected or incubated with cells and or sera and infused in situations where cost is prohibitive, and does not allow more sophisticated use of technology. Even developing Nations might gain a benefit from the technology as it is harmonized for broader and less expensive use.

All references cited herein are incorporated herein by reference in their entireties.

### BRIEF SUMMARY OF THE INVENTION

The disclosure provides a vaccine comprising: an immunoglobulin binding factor (IBF) reagent; at least one antigen or antigen immune complex (IC) reagent. The disclosure provides a vaccine wherein the IBF reagent is an Fc reagent, with collateral Fab reagent activity. The disclosure provides a vaccine wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized to yield, for example, 4 or 5 FcR-Fab reagent binding units that can be further polymerized to form 10 to 100 FcR-Fab reagent, an FcR fragmented to give, for example, less than 4 to include monovalent single (added because of difference in action MSPA) of FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof. The disclosure provides a vaccine wherein the IC reagent is selected from the group consisting of synthetic, genetically engineered, monovalent, multivalent, isolated from a patient, and combinations thereof. The disclosure provides a vaccine wherein the patient has an immune complex disease or disorder. The disclosure provides a vaccine wherein the patient has a condition selected from the group consisting of cancer, abnormal immunity to antigen, abnormal FcR mediated immunity to antigen, and combinations thereof. The disclosure provides a vaccine wherein the vaccine prevents and/or treats an immune complex disease or disorder. The disclosure provides a vaccine wherein the immune complex disease or disorder is selected from the group consisting of cancer, abnormal immunity to antigen, abnormal FcR mediated immunity to antigen, and combinations thereof. The disclosure provides a vaccine wherein the IBF reagent and IC reagent will not dissociate upon administration to a patient. The disclosure provides a vaccine wherein the binding between the IBF reagent and IC reagent is selected from the group consisting of van der Waals forces, hydrogen bonds, ionic bonds, hydrophobic interactions, and combinations thereof. The disclosure provides a vaccine wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides a vaccine wherein IBF reagent is covalently bound to IC reagent. The disclosure provides a vaccine wherein IBF reagent is covalently bound to IC reagent by a peptide, by a non-peptide linker, or combinations thereof. The disclosure provides a vaccine wherein the IBF reagent and IC reagent are covalently bound. The disclosure provides a vaccine wherein the IBF reagent and IC reagent are covalently bound by a linker. The disclosure provides a vaccine wherein the IBF reagent and IC reagent are covalently bound by a linker which is selected from the group consisting of a peptide, avidin, streptavidin, NeutrAvidin, biotin, and combinations thereof. The disclosure provides a vaccine wherein the vaccine treats and/or prevents a pathological infection. The disclosure provides a vaccine wherein the pathological infection is selected from the group consisting of bacterial infection, viral infection, fungal infection, parasitic infection, and combinations thereof.

The disclosure provides a vaccine composition comprising: At least one immunoglobulin binding factor (IBF) reagent; At least one immune complex (IC) reagent; Optionally at least one adjuvant; At least one pharmaceuticals acceptable excipient. The disclosure provides a vaccine composition wherein the IBF reagent is an Fc reagent. The disclosure provides a vaccine composition wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR polymerized to give, for example, at least 4 FcR-Fab reagent binding units, an FcR fragmented to give less than 4 FcR-Fab reagent binding units, an FcR comprising, for example, at least one but no more than 3 FcR-Fab reagent binding units, a microbial FcR mimic polypeptide, a bacterial FcR mimic polypeptide, an IgG binding bacterial polypeptide, a Staphylococcus aureus protein A, a fragment of Staphylococcus protein A, a streptococcal Protein G, a streptococcal Protein G with the albumin binding site removed, and combinations thereof. The disclosure provides a vaccine composition wherein the IC reagent is selected from the group consisting of synthetic, genetically engineered, monovalent, multivalent, isolated from a patient, and combinations thereof. The disclosure provides a vaccine composition wherein the patient has an immune complex disease or disorder. The disclosure provides a vaccine composition wherein the patient has a condition selected from the group consisting of cancer, abnormal immunity to antigen, abnormal FcR mediated immunity to antigen, and combinations thereof. The disclosure provides a vaccine composition wherein the vaccine prevents and/or treats an immune complex disease or disorder. The disclosure provides a vaccine composition wherein the immune complex disease or disorder is selected from the group consisting of cancer, abnormal immunity to antigen, abnormal FcR mediated immunity to antigen, and combinations thereof. The disclosure provides a vaccine composition wherein the IBF peptide and IC reagent will not dissociate upon administration to a patient. The disclosure provides a vaccine composition wherein the binding between the IBF reagent and IC reagent is selected from the group consisting of van der Waals forces, hydrogen bonds, ionic bonds, hydrophobic interactions, and combinations thereof. The disclosure provides a vaccine composition wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides a vaccine composition wherein IBF reagent is covalently bound to IC reagent. The disclosure provides a vaccine composition wherein IBF reagent is covalently bound to IC reagent by a peptide, by a non-peptide linker, or combinations thereof. The disclosure provides a vaccine composition wherein the IBF reagent and IC reagent are covalently bound. The disclosure provides a vaccine composition wherein the IBF reagent and IC reagent are covalently bound by a linker. The disclosure provides a vaccine composition wherein the IBF reagent and IC reagent are covalently bound by a linker which is selected from the group consisting of a peptide, avidin, streptavidin, NeutrAvidin, biotin, and combinations thereof. The disclosure provides a vaccine composition wherein the vaccine treats and/or prevents a pathological infection. The disclosure provides a vaccine composition wherein the pathological infection is selected from the group consisting of bacterial infection, viral infection, fungal infection, parasitic infection, and combinations thereof. The disclosure provides a vaccine composition wherein the adjuvant is tetanus toxin, and combinations thereof. The disclosure provides a vaccine composition wherein the at least one pharmaceutically acceptable excipient is selected from the group consisting of stabilizers, pH adjusting agents, bulking agents, buffers, carriers, diluents, vehicles, solubilizers, binders, and combinations thereof.

The disclosure provides a method of making a vaccine, wherein the vaccine comprises: An immunoglobulin binding factor (IBF) reagent; At least one immune complex (IC) reagent; the method comprising the steps of: providing an immunoglobulin binding factor (IBF) reagent; Providing at least one immune complex (IC) reagent; binding the IBF to the IC. The disclosure provides a method wherein the IBF reagent is an Fc reagent. The disclosure provides a method wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized to yield, for example, 4 or 5 FcR-Fab reagent binding units that can be further polymerized to form, for example, 10 to 100 FcR-Fab reagent, an FcR fragmented to give less than 4 to include monovalent single (added because of difference in action MSPA) of FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof. The disclosure provides a method wherein the IC reagent is selected from the group consisting of, synthetic, genetically engineered, monovalent, multivalent, isolated from a patient, and combinations thereof. The disclosure provides a method wherein the patient has an immune complex disease or disorder. The disclosure provides a method wherein the patient has a condition selected from the group consisting of cancer, abnormal immunity to antigen, abnormal FcR mediated immunity to antigen, and combinations thereof. The disclosure provides a method wherein the vaccine prevents and/or treats an immune complex disease or disorder. The disclosure provides a method wherein the immune complex disease or disorder is selected from the group consisting of cancer, abnormal immunity to antigen, abnormal FcR mediated immunity to antigen, and combinations thereof. The disclosure provides a method wherein the IBF reagent and IC reagent will not dissociate upon administration to a patient. The disclosure provides a method wherein the binding between the IBF reagent and IC reagent is selected from the group consisting of van der Waals forces, hydrogen bonds, ionic bonds, hydrophobic interactions, and combinations thereof. The disclosure provides a method wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides a method wherein IBF reagent is covalently bound to IC reagent. The disclosure provides a method wherein IBF reagent is covalently bound to IC reagent by a peptide, by a non-peptide linker, or combinations thereof. The disclosure provides a method wherein the IBF reagent and IC reagent are covalently bound. The disclosure provides a method wherein the IBF reagent and IC reagent are covalently bound by a linker. The disclosure provides a method wherein the IBF reagent and IC reagent are covalently bound by a linker which is selected from the group consisting of a peptide, avidin, streptavidin, NeutrAvidin, biotin, and combinations thereof. The disclosure provides a method wherein the vaccine treats and/or prevents a pathological infection. The disclosure provides a method wherein the pathological infection is selected from the group consisting of bacterial infection, viral infection, fungal infection, parasitic infection, and combinations thereof.

The disclosure provides a method of preventing and/or treating antigen antibody immune complex diseases and disorders in a patient, the method comprising the steps of: selecting a patient in need of preventing and/or treating antigen antibody immune complex diseases and disorders; administering to the patient a vaccine which comprises: an immunoglobulin binding factor (IBF) reagent; at least one immune complex (IC) reagent. The disclosure provides a method wherein the antigen antibody immune complex diseases and disorders involve cellular Fc receptor mediated pathological dysregulation or dysfunction of immune responses. The disclosure provides a method wherein the IBF reagent is an Fc reagent. The disclosure provides a method wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized to yield, for example, 4 or 5 FcR-Fab reagent binding units that can be further polymerized to form for example, 10 to 100 FcR-Fab reagent, an FcR fragmented to give less than 4 to include monovalent single (added because of difference in action MSPA) of FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof. The disclosure provides a method wherein the IC reagent is selected from the group consisting of, synthetic, genetically engineered, monovalent, multivalent, isolated from a patient, and combinations thereof. The disclosure provides a method wherein the patient has an immune complex disease or disorder. The disclosure provides a method wherein the patient has a condition selected from the group consisting of cancer, abnormal immunity to antigen, abnormal FcR mediated immunity to antigen, and combinations thereof. The disclosure provides a method wherein the vaccine prevents and/or treats an immune complex disease or disorder. The disclosure provides a method wherein the immune complex disease or disorder is selected from the group consisting of cancer, abnormal immunity to antigen, abnormal FcR mediated immunity to antigen, and combinations thereof. The disclosure provides a method wherein the IBF reagent and IC reagent will not dissociate upon administration to a patient. The disclosure provides a method wherein the binding between the IBF reagent and IC reagent is selected from the group consisting of van der Waals forces, hydrogen bonds, ionic bonds, hydrophobic interactions, and combinations thereof. The disclosure provides a method wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides a method wherein IBF reagent is covalently bound to IC reagent. The disclosure provides a method wherein IBF reagent is covalently bound to IC reagent by a peptide, by a non-peptide linker, or combinations thereof. The disclosure provides a method wherein the IBF reagent and IC reagent are covalently bound. The disclosure provides a method wherein the IBF reagent and IC reagent are covalently bound by a linker. The disclosure provides a method wherein the IBF reagent and IC reagent are covalently bound by a linker which is selected from the group consisting of a peptide, avidin, streptavidin, NeutrAvidin, biotin, and combinations thereof. The disclosure provides a method wherein the vaccine treats and/or prevents a pathological infection. The disclosure provides a method wherein the pathological infection is selected from the group consisting of bacterial infection, viral infection, fungal infection, parasitic infection, and combinations thereof. The disclosure provides a method wherein the vaccine is administered at a dose selected from the group consisting of about 0.1 ng to about 100 mg per day, or about 1 ng to about 10 mg per day, or about 10 ng to about 1 mg per day. The disclosure provides a method wherein the vaccine is administered to the patient on a regimen of, for example, one, two, three, four, five, six, or other doses per day. The disclosure provides a method wherein the vaccine is administered for example, for one day, two days, three days, four days, five days, six days, a week, two weeks, three weeks, four weeks, five weeks, six weeks, a month, two months, three months, four months, or more.

The disclosure provides a method wherein the dose and treatment schedule of Fc reagent is flexible, individualized and varies with different phases of the immune complex disease, and from patient to patient, being raised or lowered according to alterations in the course of the disease or the development of undesirable effects and levels of biomarkers that predict efficacy or toxicity. The disclosure provides a method further comprising the step of the administering at least one therapeutic agent to the patient. The disclosure provides a method wherein the at least one additional therapeutic agent is administered prior to, concurrently with, subsequent to, or in combination with, the vaccine.

The disclosure provides an isolated nucleic acid sequence selected from the group consisting of a nucleic acid encoding IBF, a nucleic acid encoding an IC, a nucleic acid encoding an IBF-IC, fragments thereof, variants thereof, and mutants thereof. The disclosure provides a host cell comprising a nucleic acid encoding IBF, a nucleic acid encoding an IC, a nucleic acid encoding an IBF-IC, fragments thereof, variants thereof, and mutants thereof. The disclosure provides a host cell wherein the host cell is a member selected from the group consisting of eukaryotic cells and prokaryotic cells. The disclosure provides a cell line stably transfected with a nucleic acid encoding IBF, a nucleic acid encoding an IC, a nucleic acid encoding an IBF-IC, fragments thereof, variants thereof, and mutants thereof. The disclosure provides a substantially purified polypeptide selected from the group consisting of IBF, IC, IBF-IC, fragments thereof, variants thereof, and mutants thereof.

The disclosure provides a vaccine wherein the vaccine further comprises a checkpoint inhibitor. The disclosure provides a vaccine composition wherein the vaccine composition further comprises a checkpoint inhibitor.

The disclosure provides a method of preventing and/or treating antigen antibody immune complex diseases and disorders in a patient, the method comprising the steps of: selecting a patient in need of preventing and/or treating antigen antibody immune complex diseases and disorders; administering to the patient a vaccine which comprises: At least one immunoglobulin binding factor (IBF) reagent; At least one immune complex (IC) reagent; Optionally at least one adjuvant; At least one pharmaceuticals acceptable excipient. The disclosure provides a method of preventing and/or treating antigen antibody immune complex diseases and disorders in a patient wherein the antigen antibody immune complex diseases and disorders involve cellular Fc receptor mediated pathological dysregulation or dysfunction of immune responses. The disclosure provides a method of preventing and/or treating antigen antibody immune complex diseases and disorders in a patient wherein the IBF reagent is an Fc reagent. The disclosure provides a method of preventing and/or treating antigen antibody immune complex diseases and disorders in a patient, wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized to yield 4 or 5 FcR-Fab reagent binding units that can be further polymerized to form 10 to 100 FcR-Fab reagent, an FcR fragmented to give less than 4 to include monovalent single (added because of difference in action MSPA) of FcR-Fab reagent binding units, an FcR comprising at least one but no more than 3 FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof. The disclosure provides a method of preventing and/or treating antigen antibody immune complex diseases and disorders in a patient wherein the IC reagent is selected from the group consisting of, synthetic, genetically engineered, monovalent, multivalent, isolated from a patient, a checkpoint inhibitor, and combinations thereof. The disclosure provides a method of preventing and/or treating antigen antibody immune complex diseases and disorders in a patient wherein the patient has an immune complex disease or disorder. The disclosure provides a method of preventing and/or treating antigen antibody immune complex diseases and disorders in a patient wherein the patient has a condition selected from the group consisting of cancer, abnormal immunity to antigen, abnormal FcR mediated immunity to antigen, and combinations thereof. The disclosure provides a method of preventing and/or treating antigen antibody immune complex diseases and disorders in a patient wherein the vaccine prevents and/or treats an immune complex disease or disorder. The disclosure provides a method of preventing and/or treating antigen antibody immune complex diseases and disorders in a patient wherein the immune complex disease or disorder is selected from the group consisting of cancer, abnormal immunity to antigen, abnormal FcR mediated immunity to antigen, and combinations thereof. The disclosure provides a method of preventing and/or treating antigen antibody immune complex diseases and disorders in a patient wherein the IBF reagent and IC reagent will not dissociate upon administration to a patient. The disclosure provides a method of preventing and/or treating antigen antibody immune complex diseases and disorders in a patient wherein the binding between the IBF reagent and IC reagent is selected from the group consisting of van der Waals forces, hydrogen bonds, ionic bonds, hydrophobic interactions, and combinations thereof. The disclosure provides a method of preventing and/or treating antigen antibody immune complex diseases and disorders in a patient wherein the IBF reagent and IC reagent are covalently bound. The disclosure provides a method of preventing and/or treating antigen antibody immune complex diseases and disorders in a patient wherein the IBF reagent and IC reagent are covalently bound by a linker. The disclosure provides a method of preventing and/or treating antigen antibody immune complex diseases and disorders in a patient wherein the IBF reagent and IC reagent are covalently bound by a linker which is selected from the group consisting of a peptide, avidin, streptavidin, NeutrAvidin, biotin, and combinations thereof. The disclosure provides a method wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides a method wherein IBF reagent is covalently bound to IC reagent. The disclosure provides a method wherein IBF reagent is covalently bound to IC reagent by a peptide, by a non-peptide linker, or combinations thereof. The disclosure provides a method of preventing and/or treating antigen antibody immune complex diseases and disorders in a patient wherein the vaccine treats and/or prevents a pathological infection. The disclosure provides a method of preventing and/or treating antigen antibody immune complex diseases and disorders in a patient wherein the pathological infection is selected from the group consisting of bacterial infection, viral infection, fungal infection, parasitic infection, and combinations thereof. The disclosure provides a method of preventing and/or treating antigen antibody immune complex diseases and disorders in a patient wherein the vaccine is administered at a dose selected from the group consisting of about 0.1 ng to about 100 mg per day, or about 1 ng to about 10 mg per day, or about 10 ng to about 1 mg per day. The disclosure provides a method of preventing and/or treating antigen antibody immune complex diseases and disorders in a patient wherein the vaccine is administered to the patient on a regimen of, for example, one, two, three, four, five, six, or other doses per day. The disclosure provides a method of preventing and/or treating antigen antibody immune complex diseases and disorders in a patient wherein the vaccine is administered for example, for one day, two days, three days, four days, five days, six days, a week, two weeks, three weeks, four weeks, five weeks, six weeks, a month, two months, three months, four months, or more. The disclosure provides a method of preventing and/or treating antigen antibody immune complex diseases and disorders in a patient wherein the dose and treatment schedule of Fc reagent is flexible, individualized and varies with different phases of the immune complex disease, and from patient to patient, being raised or lowered according to alterations in the course of the disease or the development of undesirable effects and levels of biomarkers that predict efficacy or toxicity. The disclosure provides a method of preventing and/or treating antigen antibody immune complex diseases and disorders in a patient further comprising the step of the administering at least one therapeutic agent to the patient. The disclosure provides a method of preventing and/or treating antigen antibody immune complex diseases and disorders in a patient wherein the at least one additional therapeutic agent is administered prior to, concurrently with, subsequent to, or in combination with, the vaccine.

The disclosure provides for the use of the compositions of the invention for the production of a medicament for treating the indications as set forth herein. In accordance with a further embodiment, the present disclosure provides a use of the pharmaceutical compositions described above, an amount effective for use in a medicament, and most preferably for use as a medicament for treating a disease or disorder in a subject. In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease in a subject.

The disclosure provides a vaccine wherein the IBF reagent is an FcR mimetic agent selected from the group consisting of complement reactive protein (CRP), complement Clq, microbial immunoglobulin binding factors (IBF), corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof. The disclosure provides a vaccine which has anti-viral activity.

The disclosure provides a method wherein the IBF reagent is an FcR mimetic agent selected from the group consisting of complement reactive protein (CRP), complement Clq, microbial immunoglobulin binding factors (IBF), corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof. The disclosure provides a method wherein the administration of a COVID-19 antigen(s) Spike (S)-protein without an IBF FcR mimetic region results in long lasting protective immunity with IBF interference. The disclosure provides a method wherein the vaccine has anti-viral activity.

The disclosure provides a vaccine comprising: an immunoglobulin binding factor (IBF) reagent; At least one antigen or antigen immune complex (IC) reagent. The disclosure provides a vaccine wherein the IBF reagent is an Fc reagent, with collateral Fab reagent activity. The disclosure provides a vaccine wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized with FcR-Fab reagent binding units that can be further polymerized to form FcR-Fab reagent, an FcR fragmented to give a plurality of monovalent single (added because of difference in action MSPA) of FcR-Fab reagent binding units, an FcR comprising at least one FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof. The disclosure provides a vaccine wherein the IBF reagent is an FcR mimetic agent selected from the group consisting of complement reactive protein (CRP), complement Clq, microbial immunoglobulin binding factors (IBF), corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof. The disclosure provides a vaccine wherein the IBF is free from attachment to immune complex (IC). The disclosure provides a vaccine wherein the IBF is free from attachment to immune complex (IC) and is selected from the group consisting of a corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof. The disclosure provides a vaccine which has anti-viral activity. The disclosure provides a vaccine wherein the IC reagent is selected from the group consisting of synthetic, genetically engineered, monovalent, multivalent, isolated from a patient, a checkpoint inhibitor, and combinations thereof. The disclosure provides a vaccine for use in preventing and/or treating an immune complex disease or disorder in a patient. The disclosure provides a vaccine for use in preventing and/or treating a condition selected from the group consisting of cancer, abnormal immunity to antigen, abnormal FcR mediated immunity to antigen, and combinations thereof, in a patient. The disclosure provides a vaccine wherein the vaccine prevents and/or treats an immune complex disease or disorder in a patient. The disclosure provides a vaccine wherein the immune complex disease or disorder is selected from the group consisting of cancer, abnormal immunity to antigen, abnormal FcR mediated immunity to antigen, and combinations thereof. The disclosure provides a vaccine wherein the IBF reagent and IC reagent will not dissociate upon administration to a patient. The disclosure provides a vaccine wherein the binding between the IBF reagent and IC reagent is selected from the group consisting of van der Waals forces, hydrogen bonds, ionic bonds, hydrophobic interactions, and combinations thereof. The disclosure provides a vaccine wherein the IBF reagent and IC reagent are covalently bound. The disclosure provides a vaccine wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides a vaccine wherein IBF reagent is covalently bound to IC reagent by a peptide, by a non-peptide linker, or combinations thereof. The disclosure provides a vaccine wherein the vaccine treats and/or prevents a pathological infection.

The disclosure provides a vaccine wherein the pathological infection is selected from the group consisting of bacterial infection, viral infection, fungal infection, parasitic infection, and combinations thereof. The disclosure provides a method of preventing and/or treating antigen antibody immune complex diseases and disorders in a patient, the method comprising the steps of: selecting a patient in need of preventing and/or treating antigen antibody immune complex diseases and disorders; administering to the patient a vaccine which comprises: an immunoglobulin binding factor (IBF) reagent; at least one immune complex (IC) reagent, thereby preventing and/or treating antigen antibody immune complex diseases and disorders in the patient. The disclosure provides a method wherein the antigen antibody immune complex diseases and disorders involve cellular Fc receptor mediated pathological dysregulation or dysfunction of immune responses. The disclosure provides a method wherein the IBF reagent is an Fc reagent. The disclosure provides a method wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized with FcR-Fab reagent binding units that can be further polymerized to form FcR-Fab reagent, an FcR fragmented to give a plurality of monovalent single (added because of difference in action MSPA) of FcR-Fab reagent binding units, an FcR comprising at least one FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof. The disclosure provides a method wherein the IBF reagent is an FcR mimetic agent selected from the group consisting of complement reactive protein (CRP), complement Clq, microbial immunoglobulin binding factors (IBF), corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof. The disclosure provides a method wherein the IBF is free from attachment to immune complex (IC). The disclosure provides a method wherein the IBF is free from attachment to immune complex (IC) and is selected from the group consisting of a corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof, wherein the IBF can engage circulating IC to increase efficacious immunity, or diminish immunity, or immune complex hypersensitivity disease. The disclosure provides a method wherein the administration of a COVID-19 antigen(s) Spike (S)-protein with an IBF FcR mimetic region results in long lasting protective immunity with IBF interference. The disclosure provides a method wherein the vaccine has anti-viral activity. The disclosure provides a method wherein the IC reagent is selected from the group consisting of, synthetic, genetically engineered, monovalent, multivalent, isolated from a patient, a checkpoint inhibitor, and combinations thereof. The disclosure provides a method wherein the patient has an immune complex disease or disorder. The disclosure provides a method wherein the patient has a condition selected from the group consisting of cancer, abnormal immunity to antigen, abnormal FcR mediated immunity to antigen, and combinations thereof. The disclosure provides a method wherein the vaccine prevents and/or treats an immune complex disease or disorder. The disclosure provides a method wherein the immune complex disease or disorder is selected from the group consisting of cancer, abnormal immunity to antigen, abnormal FcR mediated immunity to antigen, and combinations thereof. The disclosure provides a method wherein the IBF reagent and IC reagent will not dissociate upon administration to a patient. The disclosure provides a method wherein the binding between the IBF reagent and IC reagent is selected from the group consisting of van der Waals forces, hydrogen bonds, ionic bonds, hydrophobic interactions, and combinations thereof. The disclosure provides a method wherein the IBF reagent and IC reagent are covalently bound. The disclosure provides a method wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides a method wherein IBF reagent is covalently bound to IC reagent by a peptide, by a non-peptide linker, or combinations thereof. The disclosure provides a method wherein the vaccine treats and/or prevents a pathological infection. The disclosure provides a method wherein the pathological infection is selected from the group consisting of bacterial infection, viral infection, fungal infection, parasitic infection, and combinations thereof. The disclosure provides a method wherein the vaccine is administered at a dose selected from the group consisting of about 0.1 ng to about 100 mg per day, or about 1 ng to about 10 mg per day, or about 10 ng to about 1 mg per day. The disclosure provides a method wherein the vaccine is administered to the patient on a regimen of, for example, one, two, three, four, five, six, or other doses per day. The disclosure provides a method wherein the vaccine is administered for example, for one day, two days, three days, four days, five days, six days, a week, two weeks, three weeks, four weeks, five weeks, six weeks, a month, two months, three months, four months, or more. The disclosure provides a method wherein the dose and treatment schedule of Fc reagent is flexible, individualized and varies with different phases of the immune complex disease, and from patient to patient, being raised or lowered according to alterations in the course of the disease or the development of undesirable effects and levels of biomarkers that predict efficacy or toxicity. The disclosure provides a method further comprising the step of the administering at least one therapeutic agent to the patient. The disclosure provides a method wherein the at least one additional therapeutic agent is administered prior to, concurrently with, subsequent to, or in combination with, the vaccine.

### DETAILED DESCRIPTION OF THE INVENTION

The term “administration” of the pharmaceutically active compounds and compositions of the disclosure can be administered to a patient or subject by direct injection to a preselected site, systemically, on or around the surface of an acceptable matrix, or in combination with a pharmaceutically acceptable carrier. Oral, nasal, parenteral, intravenous, intramuscular, sublingual, topical, transdermal, and buccal administration is particularly preferred in the present disclosure.

“Ameliorate” or “amelioration” means a lessening of the detrimental effect or severity of the disease in the subject receiving therapy, the severity of the response being determined by means that are well known in the art.

By “compatible” herein is meant that the components of the compositions which comprise the present disclosure are capable of being commingled without interacting in a manner which would substantially decrease the efficacy of the pharmaceutically active compound under ordinary use conditions.

The terms “effective amount” or “pharmaceutically effective amount” refer to a relatively nontoxic but sufficient amount of the agent to provide the desired biological result. That result can be reduction and/or alleviation of the signs, symptoms, or causes of a disease, such as cancer. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” encompasses carriers, diluents, vehicles, solubilizers, stabilizers, bulking agents, acidic or basic pH-adjusting agents and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

As used herein, the terms “formulate” refers to the preparation of a pharmaceutical composition in a form suitable for administration to a mammalian patient, preferably a human. Thus, “formulation” can include the addition of pharmaceutically acceptable excipients, diluents, or carriers and pH adjusting agents.

By “pharmaceutically acceptable” or “pharmacologically acceptable” is meant a material which is not biologically or otherwise undesirable, i.e., the material may be administered to an individual without causing undesirable biological effects or interacting in a deleterious manner with any of the components of the composition in which it is contained.

As used herein, a “pharmaceutically acceptable carrier” is a material that is relatively nontoxic and generally inert and does not affect the functionality of the active ingredients adversely. Examples of pharmaceutically acceptable carriers are well known and they are sometimes referred to as diluents, vehicles or excipients. The carriers may be organic or inorganic in nature. In addition, the formulation may contain additives such as flavoring agents, coloring agents, thickening or gelling agents, emulsifiers, wetting agents, buffers, stabilizers, and preservatives such as antioxidants.

The term “pharmaceutical composition” as used herein means a composition that is made under conditions such that it is suitable for administration to, for example, humans, e.g., it is made under GMP conditions and contains pharmaceutically acceptable excipients, e.g., without limitation, stabilizers, pH adjusting agents, bulking agents, buffers, carriers, diluents, vehicles, solubilizers, and binders.

As used herein, the terms “patient” or “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, such as by suppressing or relieving the disease, i.e., causing regression of the disease or its clinical symptoms.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Gene activation” refers to any process that results in an increase in production of a gene product. A gene product can be either RNA (including, but not limited to, mRNA, rRNA, tRNA, and structural RNA) or protein. Accordingly, gene activation includes those processes that increase transcription of a gene and/or translation of an mRNA. Examples of gene activation processes that increase transcription include, but are not limited to, those that facilitate formation of a transcription initiation complex, those that increase transcription initiation rate, those that increase transcription elongation rate, those that increase processivity of transcription and those that relieve transcriptional repression (by, for example, blocking the binding of a transcriptional repressor). Gene activation can constitute, for example, inhibition of repression as well as stimulation of expression above an existing level. Examples of gene activation processes which increase translation include those that increase translational initiation, those that increase translational elongation and those that increase mRNA stability. In general, gene activation comprises any detectable increase in the production of a gene product, in some instances an increase in production of a gene product by about 2-fold, in other instances from about 2- to about 5-fold or any integer therebetween, in still other instances between about 5- and about 10-fold or any integer therebetween, in yet other instances between about 10- and about 20-fold or any integer therebetween, sometimes between about 20- and about 50-fold or any integer therebetween, in other instances between about 50- and about 100-fold or any integer therebetween, and in yet other instances between 100-fold or more.

“Gene repression” and “inhibition of gene expression” refer to any process which results in a decrease in production of a gene product. A gene product can be either RNA (including, but not limited to, mRNA, rRNA, tRNA, and structural RNA) or protein. Accordingly, gene repression includes those processes which decrease transcription of a gene and/or translation of a mRNA. Examples of gene repression processes which decrease transcription include, but are not limited to, those which inhibit formation of a transcription initiation complex, those which decrease transcription initiation rate, those which decrease transcription elongation rate, those which decrease processivity of transcription and those which antagonize transcriptional activation (by, for example, blocking the binding of a transcriptional activator). Gene repression can constitute, for example, prevention of activation as well as inhibition of expression below an existing level. Examples of gene repression processes which decrease translation include those which decrease translational initiation, those which decrease translational elongation and those which decrease mRNA stability. Transcriptional repression includes both reversible and irreversible inactivation of gene transcription. In general, gene repression comprises any detectable decrease in the production of a gene product, in some instances a decrease in production of a gene product by about 2-fold, in other instances from about 2- to about 5-fold or any integer therebetween, in yet other instances between about 5- and about 10-fold or any integer therebetween, in still other instances between about 10- and about 20-fold or any integer therebetween, sometimes between about 20- and about 50-fold or any integer therebetween, in other instances between about 50- and about 100-fold or any integer therebetween, in still other instances 100-fold or more. In yet other instances, gene repression results in complete inhibition of gene expression, such that no gene product is detectable.

“Modulation” refers to a change in the level or magnitude of an activity or process. The change can be either an increase or a decrease. For example, modulation of gene expression includes both gene activation and gene repression. Modulation can be assayed by determining any parameter that is indirectly or directly affected by the expression of the target gene. Such parameters include, e.g., changes in RNA or protein levels, changes in protein activity, changes in product levels, changes in downstream gene expression, changes in reporter gene transcription (luciferase, CAT, beta-galactosidase, beta-glucuronidase, green fluorescent protein (see, e.g., Mistili & Spector, Nature Biotechnology 15:961-964 (1997)); changes in signal transduction, phosphorylation and dephosphorylation, receptor-ligand interactions, second messenger concentrations (e.g., cGMP, cAMP, IP3, and Ca2+), and cell growth. These assays can be in vitro, in vivo, and ex vivo. Such functional effects can be measured by any means known to those skilled in the art, e.g., measurement of RNA or protein levels, measurement of RNA stability, or identification of downstream or reporter gene expression, e.g., via chemiluminescence, fluorescence, colorimetric reactions, antibody binding, inducible markers, ligand binding assays, and the like.

It will be appreciated that a wide variety of host cells and vectors can be used for the instant disclosure. The term “host cell” refers to one or more cells into which a recombinant DNA molecule is introduced. A “vector” is a replicon, such as a plasmid, phage, or cosmid, to which another DNA segment may be attached so as to bring about the replication of the attached segment. A DNA “coding sequence” or a “nucleotide sequence encoding” a particular protein, is a DNA sequence which is transcribed and translated into a polypeptide in vitro or in vivo when placed under the control of appropriate regulatory elements. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence.

DNA “control elements” refers collectively to promoters, ribosome binding sites, polyadenylation signals, transcription termination sequences, upstream regulatory domains, enhancers, RES (“internal ribosomal entry site”) and the like, which collectively provide for the transcription and translation of a coding sequence in a host cell. Not all of these control sequences need always be present in a recombinant vector so long as the desired gene is capable of being transcribed and translated.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence. A control element, such as a promoter, “directs the transcription” of a coding sequence in a cell when RNA polymerase will bind the promoter and transcribe the coding sequence into mRNA, which is then translated into the polypeptide encoded by the coding sequence. Operably linked may also refer to an arrangement of two or more genes encoded on the same transcript. This arrangement results in the co-transcription of the genes, i.e., both genes are transcribed together since they are present on the same transcript. This operably linked arrangement of genes can be found in a naturally occurring DNA or constructed by genetic engineering. Further, according to this operable linkage, the genes can be translated as a polyprotein, i.e., translated as a fused polypeptide such that the resultant proteins are interlinked by a peptide bond from a single initiation event, or the genes can be translated separately from independent translation initiation signals, such as an RES, which directs translation initiation of internally-situated open reading frames (i.e., protein-coding regions of a transcript.

### Therapeutic FcR IBF

During millions of years of interaction with host immune defenses infectious microbes have evolved to produce molecules that can modulate hosts' immune responses, by both immune activation and suppression, to confer survival advantage. Microbial molecules may be co-opted and used to produce beneficial drugs such as antibiotics, agents used in immunotherapy of infection and of toxins, as vaccines and adjuvants, and as anti-cancer agents.

Selective pressures acting on microbes usually favor rapid replication and efficient use of energy and substrates. The limited genomes of microbes demand evolutionary efficiency that can yield mutations in microbial proteins useful as therapeutic agents that can have multiple binding sites and additive or synergistic activities. Such therapeutic, multifunctional microbial factors include but are not limited to SPA and other microbial FcR IBF. SPA and other soluble microbial FcR IBF have many similarities and cross-reactions with eukaryotic FcR, and they can be used as effectors and for vaccines to activate antitumor responses, and also for suppressing pathological autoimmune disorders. An immunotherapy “polypharmacy” can be contained in a single microbial molecule such as FcR IBF, and microbial FcR IBF can be more efficacious than monoclonal antibody or recombinant eukaryotic IBF, and can generally be produced more cheaply. Therapeutic methods comprising administration of a soluble FcR IBF to treat a multitude of immune disorders mediated by IC and FcR, including T cell FcR mediated immunity to cancer and other antigens are described in a pioneer patent for FcR IBF pharmacology (Cowan F M. A Method for Treating Cellular Fc Receptor Mediated Immune Disorders. U.S. Pat. No. 5,189,014, Feb. 23, 1993 (original application filled Dec. 3, 1979).

### SPA as an FcR-IBF Drug

SPA is expressed by Staphylococcus aureus bacteria and is an immunoglobulin-binding factor (IBF) that binds to the Fc regions of human and animal antibodies (Eftimiadi G, Vinai P, Eftimiadi C (2017) Staphylococcal Protein A as a Pharmacological Treatment for Autoimmune Disorders. J Autoimmune Disord Vol 3:40); also reviewed in U.S. Pat. No. 5,189,014, Feb. 23, 1993 (original application filled Dec. 3, 1979). SPA and Fc receptors (FcR) compete in binding to the Fc portion of IgG, with cross-reactivity and “equivalent” actions. (Biguzzi S. Interaction of anti-staphylococcal protein A antisera with Fc receptor-bearing human normal lymphocytes. Eur J Immunol. 1979 January; 9(1):52-60). SPA is an example of an FcR IBF drug; it can both augment and inhibit T cell immunity and has demonstrated efficacy against cancer after parenteral administration in animals, and in humans by SPA extracorporeal profusion. Work on FcR IBF drugs and their pharmacology and has been described and reviewed (Cowan F M, Klein D L., Armstrong G R, Stylo, W A and Pearson J W. (1980) Fc receptor mediated immune regulation and gene expression. Biomedicine, 32, 108-110; Cowan F M. Connecting science past and future. J Chin Med Assoc 2017; 80:1-2).

### Immune Regulation by IC-FcR Interactions

Antigen-antibody immune complex (IC), as FcR ligand, has long been recognized as a key factor in immune regulation (Sinclair, N. R. StC., Lees, R. K., Elliott, E. V. 1968. Role of Fc fragment in the regulation of the primary immune response. Nature, 220, 1048 1049), immunotherapy, and cancer outcomes. IC-FcR interactions elicit positive, effector-activating or negative, inhibitory checkpoint immune responses that are antigen-specific. Most immune checkpoint inhibitors are antigen non-specific. They de-suppress and amplify these rejection or tolerance responses, contributing to either therapeutic efficacy or cancer hyperprogression. Decades ago, without benefit of recombinant DNA or monoclonal antibody technology, researchers pioneered the foundational concepts of IC antibody-Fc/FcR interaction in positive and negative immune regulation (reviewed, Cowan F M. Connecting science past and future. J Chin Med Assoc 2017; 80:1-2). The pharmacology of Fc antibody regions, anti-Fc antibody, FcR-mediated immune responses, and with respect to Coley's toxins, microbial FcR-like immunoglobulin binding factors (IBF) such as Staphylococcus protein A (SPA), were studied for immunotherapy.

Using the automobile metaphor of immunity, immunotherapies such as vaccines and adjuvants that activate immunity to antigen are analogous to stepping on the gas pedal. Stimulation of immune checkpoints suppresses immune responses, analogous to applying a brake, and checkpoint inhibitory drugs release the brake by unmasking “blocked” existing immunity to attack cancer antigens. While checkpoint inhibition can result in immunotherapy efficacy, it may also or cause side-effects that include self-antigen autoimmunity, inflammatory toxicity, tolerance immunity, and cancer hyperprogression.

Antigen-containing IC interacting with FcR can, in addition to effector and inflammatory responses, can provide either positive, antigen-specific immune activation or negative checkpoint inhibition that can peripherally influence or be influenced by antigen-nonspecific checkpoints such as PD1 and CTLA4. In accord with the automobile metaphor, IC-FcR interactions are analogous to the transmission that regulates whether immunity to an antigen is in [D] drive—active immunity, in [N] neutral—no response, or in [R] reverse—immune suppression and possible hyperprogression. IC-FcR interactions and IBF soluble checkpoint receptor immunotherapy has been presented by many investigators as fertile ground for developing potent immune checkpoint modulating drugs and therapeutic methods.

In certain embodiments as disclosed here, the checkpoint is an antibody checkpoint that binds to antibody Fc or Fc receptor (FcR) defining a specific checkpoint receptor site:

Antibody Checkpoints can consist of or Fc and Fab Receptor/Reagent FcR mimetic peptides or drug as set forth herein. This can include many types of FcR mimetic molecule such expressed by complement reactive protein (CRP), complement Clq, microbial immunoglobulin binding factors (IBF) like Staphylococcus protein A (SPA) and some corona virus spike (S) protein FcR mimetic, that are very like SPA the prototype IBF used in my original pioneer application. Microbes use these immunoglobulin binding factors to subvert/jam host immunity to allow their replication and this can cause collateral pathology that looks like antibody mediated autoimmunity or immune complex hypersensitivity disease. IBF can be usurped for immunotherapy OR play hell and havoc with vaccine or treatment R&D. Corona virus make about 30 proteins playing one of these limited deck of genic cards as an antibody binding checkpoint is not without mechanistic consequence.

Certain bacteria, virus and other microbes genetically evolved Fc receptor (FcR) immunoglobulin binding factor (IBF), also called FcR mimetics, to influence FcR antibody checkpoints and effector functions. Such IBF can in addition to Fc antibody (FcA) sometimes also contain an antibody Fab binding site or other binding site such as an albumin binding site. Corona virus spike proteins can contain both IBF and receptor for angiotensin-converting enzyme 2 (ACE2) infectious receptor. Many drugs molecules and drugs “Have multiple sites of action and/or pharmacology” as stated herein. Microbial IBF have evolved to cause immune subversion and redirection of immunity to protect microbial survival and replication and can collaterally cause pathology. A role for immune complex (IC) engaging cellular FcR and soluble FcR IBF in immune regulation, effector or checkpoint and/or the pathology and treatment of infection has long been predicted and demonstrated.

The prototype microbial IBF Staphylococcus protein A (SPA) discovered near a century ago has been usurped for immunotherapy to include treatment of infection and cancer. IC has long been associated with cancer pathology; however, so far no one has checked SARS-CoV-2 for IBF or done commercially available circulating IC assays in COVID-19 patients. IC and IBF can have major implications for pathology, treatment and vaccination. The microbial IBF SPA has defeated vaccine development to this microbe. The number of binding sites for FcR or valance has proved critical to FcR inflammatory pathology for microbial bacterial SPA IBF anaphylactoid/anaphylaxis and Arthrus reactions. For example, monovalent SPA MSPA can inhibit polyvalent SPA IBF IC or antibody mediated aggregation and IC disease. A single FcR cite binding peptide such as monovalent SPA (MSPA) can inhibit multi-FcR site SPA microbial antibody aggregation and FcR inflammation. The method of the present invention is using FcR protein/peptide or FcR binding drug to block, inhibit, or diminish viral-IBF inflammation and immunity in a patient in need of such immunotherapy. Other species of molecule that can be used as monomers of their active site are CRP and Clq and isolated segment of corona virus spike (S)-protein containing IBF or drugs that bind either Fc antibody or FcR.

The new corona virus SARS-CoV-2 causes COVID-19 associated pathology that has ALL the hallmarks of immune complex disease complicated by viral IBF. Further such IC and IBF immune dysfunction can explain both high risks of age and comorbidities and prolonged immune dysfunction and pathology following COVID-19 “long haulers.” Many corona virus code for and produce IBF. Singularly or more potently collectively IC and/or IBF can cause Arthrus like reactions that focus inflammatory pathology to sites of infection and/or collaterally influence immune regulation, effector function or even destroy antigen specific immune cell clones that are responding to the viral antigen contained in the IC.

FcR can be cellular or circulate as soluble IBF and microbial IBF can be associated with the microbe surface membrane or capsid or shed in soluble form. Both attached and soluble FcR or IBF are “antibody” checkpoints of immunity that can influence both innate and antigen specific adaptive inflammation, immunity and immune regulation. Checkpoints are described as both the brake and accelerator of immune response and FcR/IBF antibody checkpoints as the transmission that determines whether the immunity is in driver [D] rejection, neutral [N] or reverse [R]. Most other checkpoints are influenced by FcR mediated immunity. FcR are can function as a potential antigen specific checkpoint of checkpoints. Such powerful immune balance of tolerance and rejection has been have used by microbes to enhance their replication and usurped for immunotherapy to augment or inhibit both tolerance rejection to antigens for immunotherapy of cancer and autoimmunity other diseases. Microbes have evolved by mutation or captured by recombination IBF to enhance survival against host immunity to insure their replication.

The pathology of coronavirus such as severe acute respiratory syndrome (SARS) and COVID-19 (SARS-CoV-2) involves hypercytokinemia, “cytokine storm” induced ARDS that develops in a time fame consistent with host antibody response and higher antibody titers are associated with critical/pathological outcomes. Some coronavirus used their limited genome to code for an IBF on “spike” S-protein. A viral IBF polymerized on the virus “coat” or infected cell that binds IC would cause an IC-FcR mediated Athrus-like pseudo-immune reaction or anaphylactoid shock, contributing to development of hypercytokinemia and ARDS and the many other immune complex disease-like pathology associated with SARS-CoV-2. This would involve the complement system Clq, complement reactive protein (CRP) and antibody Fc and FcR. All these components species can involve multivalent aggregation of antibody/immunoglobulin (Ig) to activate either physiological or pathological inflammation by antibody mediated innate or adaptive antigen mediated immunity. Monomers of Clq, CRP and antibody Fc and FcR/IBF peptides or chemical compound drugs can provide a method to reduce aggregation and associated pathological immunity. Pathology of SARS-CoV-2 would occur as antiviral antibody formed IC that bound to virus IBF and created inflammatory foci in the lungs that cause inflammation cumulating in ARDS or other IC pathology.

Thus, microbial IBF binding to antibody Fc region and can cause a pseudo immune complex disease (Type II hypersensitivity reaction) (Athrus-like pseudo-immune reaction) that can produce the broad spectrum of pathology and select for this pathology in susceptible patient groups associated with SARS-CoV-2.

Antigen antibody immune complex (IC) and soluble and cellular FcR mediated immunity can be generated in a concentration dependent manner of these few “foundational” basic participants. This can be understood as receptor-ligand interactions. There are hundreds of receptors and their respective ligands. However, this one system is central and can predict much of the result of the peripheral complexity to more elementary concentration [C] dependent reactions.

COVID-19, the disease caused by SARS-CoV2 infection, has all the hallmarks of immune complex hypersensitivity disease. COVID-19 infection patients who have severe pathology, it generally appears in a time-frame consistent to development of antibody that can form immune complex (IC) that interact with viral IBF, soluble or cellular FcR to mediated immunity and EVERY class of contraindicated patient (age, hypertension, diabetes, obese etc. has propensity for increased autoimmune IC and/or immune dysfunction that fails to more efficiently clear IC, predisposing to IC-FcR mediated inflammation. Coronavirus Viral IC can further infect cells through FcR. There are four types of immune hypersensitivity reaction. A “modified” type III reaction that is killing patients. (See www.bing.com/videos/search?q=type+III+hypersensitivity&&view=detail&mid=2DD4D2CBD EB4BD85C7262DD4D2CBDEB4BD85C726&&FORM=VRDGAR&ru=%2Fvideos%2Fsearch%3Fq%3Dtype%2BIII%2Bhypersensitivity%26FORM%3DHDRSC3). This is also in keeping with symptoms/pathologies effecting lungs and other organs brain, liver, heart, skin and systems coagulation and inflammation associated symptoms hypercytokinemia, ARDS and immune complex disease.

Implications: This type of IC disease pathology has proved rather refractory to interventions. Since antibody binding to FcR can increase infectivity or pathology, vaccines must be considered with great caution. One would not want to make young and healthy person more susceptible to this virus. Some coronavirus enhance the danger of type III hypersensitivity that also provide a target for therapy.

The pathology of coronavirus, such as SAR and COVID-19, IC disease would involve the complement system Clq, CRP and antibody Fc and FcR. All these components involve multivalent aggregation of antibody/Ig to activate either physiological or pathological inflammation by antibody mediated pseudo-immune and/adaptive antigen mediated immunity. Monomers of Clq, CRP and antibody Fc and FcR/IBF peptides or chemical compound drugs can reduce aggregation and associated pathological immunity. Pathology of COVID-19 would occur as antiviral antibody formed IC that bound to virus IBF and created inflammatory foci in the lungs or other organs that cause inflammation cumulating in ARDS. Other sources of IC collateral infection, autoimmune IC, or even IC and or IBF generated immune influences could have both short and long term effects on immune pathology or immunotherapy.

The disclosure provides for the use of vaccination with a COVID-19 antigen(s) such as spike (S)-protein with, for example, IBF FcR mimetic activity absent or removed, so that long lasting protective immunity can be achieved with IBF interference.

Immunological concentration [C] dependent destination of Arthus reaction is analogues to a dust-initiator explosion in a grain silo where just the right concentration can cause devastation/pathology. COVID-19 mortality and morbidity as an immune complex disease type III and preferably-peripheral treatments that influence IC disease can encompass anti-viral, anti-inflammatory or anti-coagulant drugs and/or plasmapheresis, IVIg or specific anti-viral antibody sera factor treatments to alter the concentration of “participants” to less pathological configuration. It is a particular advantage that IVIg and heparin has been used to treat COVID 19.

In addition, for COVID-19 is patients with the higher antibody levels are having the most critical or fatal outcomes. Perhaps vaccines that increase antibody or antibody therapy that can form immune complex should be considered with greater caution. Antibody can bind viral antigen and neutralize a virus OR form antigen-antibody immune complex that are too small for the immune system to clear that when deposited in tissue cause immune complex hypersensitivity disease that can affect any system or organ, just/exactly like COVID-19 pathology. Corona virus for decades and COVID-19 more recently “Are” predicted to cause immune complex hypersensitivity disease with some supporting data in the science and patent literature.

Antibody drugs might best be given early on in infection to neutralize the virus before the patient mounts their own antibody response. Dexamethasone is given late to quell pathological inflammation. Timing is critical in immunotherapy it is not just what you do, but when you do it. For example, if an antibody drug that forms immune complex is given when a COVID-19 patient is already experiencing immune complex hypersensitivity disease, a negative outcome may result. Looking at antibody titer and especially circulating immune complex levels, even immunoglobulin levels might be wise before giving an antibody drug that can form immune complex. Further with high antibody titer as a biomarker of COVID-19 pathology vaccines should be studied will before broad application. For example, rituximab was associated with significantly higher risk of hospitalization, ICU admission, and ventilation. Weaker but similar associations were seen for ocrelizumab, and these did not always reach statistical significance. Data has shown that when pooled anti-CD20 DMTs were compared with other DMTs. MS patients on anti-CD20 drugs were 1.5 times more likely to be admitted to the hospital (aPR 1.49), 2.6 times more likely to be admitted to the ICU (aPR 2.55), and 3.1 times more likely to need artificial ventilation (aPR 3.05, P<0.05 for all) than those using other drugs. (Some MS Drugs Linked With Worse COVID-19 Outcomes Registry data looks at risks of hospitalization, ICU admission, ventilation by Judy George, Senior Staff Writer, MedPage Today Sep. 28, 2020, www.medpagetoday.com/meetingcoverage/ectrims/88842?utm_source=Sailthru&utm_medium=email&utm_campaign=Weekly %20Review %202020-10-04&utm_term=NL_DHE_Weekly_Active In addition, the evidence demonstrates that in COVID-19 vasculitis a life-threatening escalation from type 2 T-helper immune response (humoral immunity) to type 3 hypersensitivity (immune complex disease) takes place. The subsequent deposition of immune complexes inside the vascular walls is supposed to induce a severe inflammatory state and a cytokine release syndrome, whose interleukin-6 is the key myokine, from the smooth muscle cells of blood vessels. Type 3 hypersensitivity in COVID-19 vasculitis. LucaRoncatia, et al. doi.org/10.1016/j.clim.2020.108487.

There is evidence that binding of antibodies to viral antigens can create antigen-antibody immune complexes, which, if not eliminated in certain individuals having dysfunctional complement systems, can start inflammatory type III hypersensitivity symptoms, including protease releases that can disrupt epithelium, mesothelium, and endothelium basement membranes, and induce pervasive inflammation throughout the body. This could continue after SARS-CoV-2 infections end if the first wave of protease attacks on basement membranes created new secondary autoantibodies and new uncleared antigen-antibody immune complexes.

There is molecular mimicry between the S peplomer protein of Mouse Hepatitis Virus (MHV) and Fc gamma Receptor (Fc gamma R) of IgG. Rabbit IgG, but not its F(ab′)2 fragments, monoclonal rat and mouse IgG and the rat 2.4G2 anti-mouse Fc gamma R monoclonal antibody (mab) immunoprecipitated natural and recombinant MHV S protein. In addition, MHV S peplomer protein exhibits Fc IgG binding ability. There is also molecular mimicry between the S peplomer protein of Bovine Coronavirus (BCV) and Fc gamma R. BCV S peplomer protein which belongs to the same antigenic subgroup as MHV also binds Fc portion of rabbit IgG and is immunoprecipitated by the 2.4G2 anti-Fc gamma R mab. (Oleszak E L, Perlman S, Parr R, Collisson E W, Leibowitz J L. Molecular mimicry between S peplomer proteins of coronaviruses (MHV, BCV, TGEV and IBV) and Fc receptor. Adv Exp Med Biol. 1993; 342:183-8). Molecular mimicry between S peplomer protein of MHV-JHM and Fc gamma R has been extended to other members of Coronaviridae, namely bovine coronavirus and transmissible gastroenteritis virus but not to infectious bronchitis virus. The molecular mimicry of viral antigens to Fc receptors has been described also for members of Herpesviridae, namely Herpes simplex, cytomegalovirus and Varicella zoster. (Arch Immunol Ther Exp (Warsz). 1994; 42(2):83-8. Molecular mimicry between Fc receptors and viral antigens. Oleszak ELi).

The effect of C Reactive Protein (CRP) on immune cells is mediated by the activation of complement and the activation of cell surface receptors. CRP was previously thought to bind to specific CRP receptors on myeloid cells. However, more recent studies suggest that CRP interacts with the same receptors used by IgG, the FcγR. The binding of CRP to FcγR is expected to activate the same signaling pathways as the binding of IgG. Binding to FcγR containing immunoreceptor tyrosine-based activation motifs, such as FcγRI, FcγRIIa/c and FcγRIIIa, would be expected to cause activation leading to inflammatory cascades, while binding to FcγRIIb, which contains an immunoreceptor tyrosine-based inhibitory motif, leads to anti-inflammatory responses. As FcγR signaling is at the interface of CRP's interaction with the immune system, these interactions are responsible for many of the immunoregulatory activities of CRP. C-reactive Protein at the Interface Between Innate Immunity and Inflammation Andres Peisajovich; Lorraine Marnell; Carolyn Mold; Terry W Du Clos. Disclosures Expert Rev Clin Immunol. 2008; 4(3):379-390. www.medscape.com/viewarticle/575924_4.

Mortality is a word that refers to casualties or deaths resulting from disease or any calamity. Even in an outbreak of an epidemic, the seriousness or intensity of the disease is judged on the basis of mortality rates. Morbidity, on the other hand is a word that is associated with sickness or illness.

Comorbidity refers to the simultaneous presence of two chronic diseases or conditions in a patient. “the comorbidity of anxiety and depression in Parkinson's disease” Another antibody checkpoint is FcR binding ligand consisting of an antibody Fc portion or mimic peptide or drug of said antibody Fc region that binds to Fc receptor as an antibody-ligands.

### Biomarkers and Assays

Due to their central role in IC-Fc/FcR interactions as mediators of both innate and adaptive immune responses, variation in types and concentrations of a patient's IC can serve as “biomarkers” that are be predictive of immunotherapy efficacy or tumor hyperprogression. For each patient, some but not extensive assays of the patient's medical and immunological status, comprising collection, analysis, and processing of clinical samples, e.g., “liquid biopsies” of blood/sera, can identify disease parameters and type and concentration of IC. Assay results permit better determination of the type and dosage range of IBF to be provided for efficacy, and can reduce the unpredictability, inconsistency and toxicity now associated with immunotherapy. This prognostic utility can be independent of the type of immune checkpoint inhibitor, either antigen-specific or non-specific, or associated differences in FcR binding, that is used to overcome immune suppression.

Examples of IC-FcR interaction in immunotherapy efficacy, and new methods for using IC-receptor interactions to elicit antigen-specific immune responses for which IC assay can be predictive of outcome include and are not limited to: (a) IC-induced dendritic cell (DC) maturation in vitro that enables DCs to prime peptide-specific CD8⁺ CTLs in vivo (Schuurhuis D H, Ioan-Facsinay A, Nagelkerken B, van Schip J J, Sedlik C, Melief C J, Verbeek J S, Ossendorp F. Antigen-Antibody Immune Complexes Empower Dendritic Cells to Efficiently Prime Specific CD8+ CTL Responses In Vivo. J Immunol 2002; 168:2240-2246); and b) infusion of donor mononuclear cells into chronic myelogenous leukemia (CML) patients to generate plasma IC containing nucleic acids that engage innate immune sensors and provide long-lasting tumor remissions (Lin Y, Zhang L, Cai A X, Lee M, Zhang W, Neuberg D, Canning C M, Soiffer R J, Alyea E P, Ritz J, Hacohen N, Means T K, Wu C J. Effective posttransplant antitumor immunity is associated with TLR-stimulating nucleic acid-immunoglobulin complexes in humans. J Clin Invest 2011; 121:1574-1584).

In addition, it has been reported that members of the pentraxin family such as C-reactive protein (CRP) and serum amyloid P component (SAP) “can crosslink and activate a subgroup of Fc receptors upon opsonization, similar to Fc receptor activation by immune-complexes.” (Lu J, Mold C, Du Clos T W, Sun P D. Pentraxins and Fc Receptor-Mediated Immune Responses. Frontiers in Immunology|www.frontiersin.org 2 Nov. 2018 Volume 9 Article 2607). Through their interactions with FcR, pentraxins such as CRP and SAP can also have utility as biomarkers to pathology and immunotherapy efficacy.

### Immune Complex Vaccines:

“From therapeutic antibodies to immune complex vaccines” by Xuan-Yi Wang, Bin Wang and Yu-Mei Wen in npj Vaccines (2019) 4:2; Also see (Wen Y M, Mu L, Shi Y. Immunoregulatory functions of immune complexes in vaccine and therapy. EMBO Mol Med. 2016 Oct. 4; 8(10):1120-1133. doi: 10.15252/emmm.201606593. Print 2016 October) doi.org/10.1038/s41541-018-0095-z) the authors highlight recent research on the use of immune complex (IC)-Fc interactions with FcR for immunotherapy, especially IC vaccines, a subject relating to the methods for IC-IBF vaccine-effector-checkpoint-adjuvant molecular complex claimed herein. Quote, “This review summarizes the background, the mechanistic studies on Fc-FcR functions, the translational research on Fc-FcR and the prospects of IC-based vaccines,” The authors imply that progress on understanding “Fc-FcR interactions” to achieve pharmacology was possible only after more recent innovations. The discovery of IC-FcR interactions and FcR IBF pharmacology relating to IC vaccines have deeper roots.

### IBF-IC Vaccine

It is a particular advantage of the disclosure that a construction of IBF-IC can address the pathology of any antigen bound to an IC and can treat for example, both tolerance and autoimmune components of an IC disease with associated abnormal FcR checkpoint or immunity (Yun Lin, Li Zhang, Ann X. Cai, Mark Lee, Wandi Zhang, Donna Neuberg, Christine M. Canning, Robert J. Soiffer, Edwin P. Alyea, Jerome Ritz, Nir Hacohen, Terry K. Means, Catherine J. Wu. Effective posttransplant antitumor immunity is associated with TLR-stimulating nucleic acid-immunoglobulin complexes in humans. The Journal of Clinical Investigation 121-4, 1574-84, 2011), (Zengguang Xu, Fengying Wu, Chunhong Wang, Xiyu Liu, Baoli Kang, Shan Shan, Xia Gu, Kailing Wang, Tao Ren. The stimulatory activity of plasma in patients with advanced non-small cell lung cancer requires TLR-stimulating nucleic acid immunoglobulin complexes and discriminates responsiveness to chemotherapy. Xu et al. Cancer Cell International 2014, 14:80 www.cancerci.com/content/14/1/80).

The IBF of the IBF-IC vaccine of the disclosure may, for example, provoke an adjuvant action that presents the IC vaccine of the patient's IC containing neoantigens to antigen presenting cells (APC) that activate T cell immunity that destroys, for example, cancer cells. Wherein, a targeted antigen specific individual immune response to the patient's cancer can be delivered without more major and expensive immune manipulation or very expensive monoclonal or vaccine or more extensive cell population ex vivo culture. If such expensive additional immunotherapy is further required, IBF-IC could result in lower doses and shorter duration of other expensive immunotherapy reducing costs and increasing efficacy. It is a particular advantage of the present disclosure that other forms of immunotherapy such as other vaccination and other checkpoint drugs can increase baseline IC levels so that responder populations to IBF-IC treatment can be increased.

In certain aspects as disclosed herein, the IBF-IC vaccine for example, and can be the result of immunizing with an exogenous tumor antigen. However, in certain aspects, the IBF-IC vaccine may be derived using a patient's own existing immunity to endogenous (self) tumor or cancer-neoantigens, that are in the patient's own sera. And which contain cancer-antigen antibody IC to build an individualized vaccine to promote efficacy. (Wirth T C, Kühnel F. Neoantigen Targeting-Dawn of a New Era in Cancer Immunotherapy? Front Immunol. 2017; 8: 1848. Published online 2017 Dec. 19). In certain aspects of the disclosure, a patient's neoantigen may be derived from, for example, KRAS, p53, BRAF, JAK2, histone H3, isocitrate dehydrogenase-1, Dpagt1, Reps1, Adpgk, PD-1, CD96, and CD137. Hundreds of tumor and cancer neoantigens have been identified. An aspect of the disclosure does not require identification, just isolation and application of the patients IC to form the IC-IBF vaccine. Cancer immunity is very individual and efficacy may require a method that takes this into account.

One critical embodiment/aspect of the invention is the number of binding sites: for example, IBF “valance” can be singular “monovalent” or multivalent for antigens, IC, IBF and linkers. The number of active sites per each ligand or receptor molecular element of the disclosure to include the antigen and antibody that forms the IC vaccine, IBF adjuvant FcR-Fab reagent and the biotin avidin, streptavidin, and “NeutrAvidin” linkers can all have singular or multiple binding sites. The molecular “lattice” or these combined reactions can form in smaller or larger complex/aggregates. Large complexes would be more quickly phagocytized by antigen presenting cells and presented to T regulatory cells for active immunity; whereas smaller even single antigenic determinant hapten complexes could desensitize T cells producing tolerance reflective of the two way street of tolerance and immunity in homeostasis and pathology of the immune the response (Voisin 1971).

The systemic administration of IBF or IBF-IC can be directly administered, or modified by incubated with cell and sera fractions separated by know method (David S. Terman PROTEIN A AND STAPHYLOCOCCAL PRODUCTS IN NEOPLASTIC DISEASE. CRC Critical Reviews in Ontology˜Hematology Volume 4, Issue 2 103-124, 1985). U.S. Pat. No. 5,189,014 (Cowan) describes using Fc receptor (FcR) to treat diverse pathologies having an FcR-mediated immune component, including bacterial and viral infections, autoimmune disorders, transplant rejection, and cancer. This patent and information derived from earlier studies and their related publications helped to pioneer the field of receptors as drugs and establish the role of FcRs as effectors of immune regulation that can influence gene expression, cellular differentiation, viral replication, and malignant transformation. The field of FcR-based drugs remains a focus of contemporary science long after the original discovery (Fred M. Cowan. Editorial Connecting science past and future. Journal of the Chinese Medical Association Volume 80, Issue 1, January 2017, Page 1-2. authors.elsevier.com/sd/article/S1726-4901(16)30184-8).

The decades breach in immunotherapy of cancer interest and funding has left a disconnect of much information generated prior to more recent renewed interest in the subject, to include the relative roles FcR IBF and IC. “Previous work on FcR immunotherapy suggested a broad range of immune regulatory effects on endogenous FcR-mediated immunity, and potential therapeutic effects in many diseases with associated abnormal FcR-mediated immunity.3,4 For example, the early work by Coley showed antineoplastic activity of bacterial extracts likely to have contained FcR-like proteins.⁶ Interestingly, sera perfused through SPA columns were observed to have antineoplastic activity in animal models and human patients, but the antineoplastic action of the perfused sera was lost as the SPA column technology improved to provide better binding of SPA to the support matrix and less leaching of SPA. Systemic injection of SPA in animal models also produced antineoplastic results. However, to my knowledge, the concept of systemic SPA-FcR immunotherapy with known injected doses was never tested clinically in human patients. At the time of our early publications, only SPA-FcR activity was known and proposed as the best mode of action.³ Other binding actions of SPA such as Fab antibody “superantigen” are now known. Many of the broad observations we made could be caused by or influenced synergistically by binding mechanisms other than SPA-FcR. SPA deserves new study that brings together past discovery and modern technology. Exciting progress in this direction has been made by Protalex, Inc., which is developing immunotherapies using MSPA to treat pathological inflammatory responses and autoimmune diseases” (Fred M. Cowan. Editorial Connecting science past and future. Journal of the Chinese Medical Association Volume 80, Issue 1, January 2017, Page 1-2. authors.elsevier.com/sd/article/S1726-4901(16)30184-8).

It is a particular advantage of the present disclosure of IBF-IC vaccine that one embodiment can use an individual patients own immune complex without excessive experimentation well within the skill of any good clinical laboratory or drug manufacturer. In one embodiment of the disclosure the patient's immune complex can be harvested from their blood sera and screened for interaction with different IBF to determine the best IBF and doses of said IBF for the patient's type and concentration of immune complex and immune cells to achieve the best clinical outcome. It is a particular advantage of this procedure that the patient's own peripheral blood immune cells such as antigen presenting cells and regulatory T and B lymphocytes can be assayed or further isolated separated with the bound IBF-immune complex adjuvant vaccine to determine best application and doses of the disclosure to each individual patient. Further this antigen specific immunity can be further enhanced by other antigen nonspecific checkpoint regulators or blockers or other antigen specific vaccines. Without a “baseline” level of antigen specific immunity to cancer antigen no “active” immunotherapy can work; although passive immunotherapy can still function. This disclosure is specifically designed to induce or enhance such baseline immunity. This will allow more successful application of other active or passive immunotherapy and perhaps break the stalemate of low response rates for major systemic cancers (Brendon J Coventry, Martin L Ashdown. Complete clinical responses to cancer therapy caused by multiple divergent approaches: a repeating theme lost in translation. Cancer Management and Research 2012:4 137-149). Such synergy between immunotherapy is proposed for achieving better efficacy (reviewed Cowan, JCMA 2017, Parchment, et al. Semin Oncol. 2016 August; 43(4):501-13. Epub 2016 Jun. 16. Immuno-pharmacodynamics for evaluating mechanism of action and developing immunotherapy combinations doi:10.1053/j.seminoncol.2016.06.008.

### Cancer Immunotherapy

The cancer triad of surgery, chemotherapy, radiation has added a forth leg of immunotherapy. While this has seen more recent success with check point blockers giving complete responses in a minority of patients and facilitating potentially lethal autoimmune or cancer hyperprogression side effects in a lesser minority of patients. Coventry suggests that all forms of conventional triad for metastatic cancer therapy have immunotherapeutic actions in addition to removal or cytotoxicity to cancer cells. The remission of cancers associated with microbial infection and causing infection to treat cancer with some success and much danger has been noted since ancient Egyptian physician Imhotep (c 2600 BC) (Jessy T. Immunity over inability: The spontaneous regression of cancer. J Nat Sc Biol Med 2011; 2:43-9). Coley toxins around the 1890s-1900s Used safer attenuated microbial extracts that could have contained proteins with FcR-Fab IBF activity (Cowan, F. M. A Method for Treating Cellular Fc Receptor Mediated Immune Disorders. U.S. Pat. No. 5,189,014, Feb. 23, 1993 (original application filled 1979), (Cowan, F. M., Klein, D. L., Armstrong, G. R., Stylos, W. A. and Pearson, J. W. (1980). Fc receptor mediated immune regulation and gene expression. Biomedicine, 32, 108-110). (Jessy T. Immunity over inability: The spontaneous regression of cancer. J Nat Sc Biol Med 2011; 2:43-9). “Stimulated immunotherapies ran a natural death in the latter half of the 20th century due to a number of reasons.

First, with the newer concept of asepsis, cancer surgery like any other operation became a sterile procedure with fewer postsurgical infections especially after Lister's aseptic techniques in the late 1800s. Second, by the time of Coley's death in 1936, radiotherapy was an established treatment for cancer and chemotherapy was slowly gaining acceptance. Such therapies though highly immunosuppressive could more easily be standardized than Coley's approach. Third, the administration of antibiotics further reduced the incidence of postsurgical infections and antipyretics came into routine use to eliminate fever and discomforting symptoms of an immune response, and the lastly due to an unfavorable approach of the medical industrial regulatory complex of the 1960s. Cancer therapies have been standardized and have improved since Coley's day, but these improvements in treatment have resulted for the most part in prolonging the disease rather than curing it.” Only 7% of systemic metastatic cancers achieve complete responses to any therapy (Brendon J Coventry, Martin L Ashdown. Complete clinical responses to cancer therapy caused by multiple divergent approaches: a repeating theme lost in translation. Cancer Management and Research 2012:4 137-149).

The importance of vaccination, immune surveillance of cancer cell, development of monoclonal antibody and receptor-ligand interaction such as FcR and immune complex are noted as key to immunotherapy of cancer and monoclonal anti-receptor check point regulators. However, the concept of using receptors or their mimics such as anti-ligand monoclonal antibodies or microbial receptor mimics has been less visible. This despite the fact that cellular receptor are the core of immunology and their interaction with antigen or other ligands the driving force of either tolerance or rejection. These abstractions where apparent to Ehrlich over a hundred years ago who also considered immunotherapy “magic bullets” against cancer. Despite the fact that immunotherapy of cancer was stalled for a few decades receptor drugs to increase tolerance and reduce autoimmune disease have been developed and used clinically as drugs for decades (Enbrel, Etanercept. Earlier Microbial FcR such as SPA where used in Immunotherapy of cancer (reviewed Cowan JCMA 2017, Cowan U.S. Pat. No. 5,189,014. The decades of a dearth of support for cancer immunotherapy also stalled this technology. However, again these molecules were further developed for autoimmune disease treatments (Protalex) a use described in the original patent (Cowan, F. M. A Method for Treating Cellular Fc Receptor Mediated Immune Disorders. U.S. Pat. No. 5,189,014, Feb. 23, 1993 (original application filled 1979).

The present disclosure also provides SPA fixed to -antigen or -immune complex and cancer immunotherapy; however, other sources of Immunoglobulin Binding Factor (IBF) may be used and other immune diseases with positive or negative immune dysfunction will benefit from this exemplary not limiting technology. The immune response is complex and variable in specific positive rejection or negative tolerance response and intensity of response; however, the basic immune mechanisms are the same for all antigens, self such as cancer, autoimmunity or external antigens, microbial, grafts or both foreign and self as in a fetus. This premise and experimental evidence allows the broad disease claims in the pioneer (U.S. Pat. No. 5,189,014, Cowan 1993, filed 1979) and present disclosure that include cancer in the pioneer FcR patent. The basic precepts of FcR immunity and immune regulation have been known for decades and where cited in the application. More recent publications further support the concepts put forth in the pioneer U.S. Pat. No. 5,189,014 filed in 1979 and issued 1993) (see references list).

Another aspect of the disclosure relates to a method for inducing an immunological response in an individual, particularly a mammal which comprises inoculating the individual with, for example, IBF and/or IC, an IBF-IC vaccine, or a fragment or variant thereof, adequate to produce antibody and/or T cell immune response to protect said individual from a pathogenic infection, such as a viral or bacterial infection. Another aspect of the disclosure relates to a method for inducing an immunological response in an individual, particularly a mammal which comprises inoculating the individual with, for example, IBF and/or IC, an IBF-IC vaccine, or a fragment or variant thereof, adequate to produce antibody and/or T cell immune response to protect said individual from a condition, for example, cancer. Also provided are methods whereby such immunological response slows viral and/or bacterial replication. Yet another aspect of the disclosure relates to a method of inducing immunological response in an individual which comprises delivering to such individual a nucleic acid vector to direct expression of, for example, IBF and/or IC, or a fragment or a variant thereof, for expressing, for example, IBF and/or IC, an IBF-IC vaccine, or a fragment or a variant thereof in vivo in order to induce an immunological response, such as, to produce antibody and/or T cell immune response, including, for example, cytokine-producing T cells or cytotoxic T cells, to protect said individual from disease, whether that disease is already established within the individual or not. One way of administering the gene is by accelerating it into the desired cells as a coating on particles or otherwise. Such nucleic acid vector may comprise DNA, RNA, a modified nucleic acid, or a DNA/RNA hybrid.

A further aspect of the disclosure relates to an immunological composition which, when introduced into an individual capable or having induced within it an immunological response, induces an immunological response in such individual to, for example, a IBF and/or IC gene, an IBF-IC encoding nucleic acid, or protein coded therefrom, wherein the composition comprises, for example, a recombinant IBF and/or IC gene or protein coded therefrom comprising DNA which codes for and expresses an antigen of said IBF and/or IC, an IBF-IC vaccine encoding nucleic acid, or protein coded therefrom. The immunological response may be used therapeutically or prophylactically and may take the form of antibody immunity or cellular immunity such as that arising from CTL or CD4+T cells.

In an exemplary embodiment, an IBF and/or IC polypeptide, an IBF-IC vaccine polypeptide or a fragment thereof may be fused with co-protein which may not by itself produce antibodies, but is capable of stabilizing the first protein and producing a fused protein which will have immunogenic and protective properties. Thus fused recombinant protein, preferably further comprises an antigenic co-protein, such as lipoprotein D from Hemophilus influenzae, Glutathione-S-transferase (GST) or beta-galactosidase, relatively large co-proteins which solubilize the protein and facilitate production and purification thereof. Moreover, the co-protein may act as an adjuvant in the sense of providing a generalized stimulation of the immune system. The co-protein may be attached to either the amino or carboxy terminus of the first protein.

Also, provided by this disclosure are methods using the described polynucleotide or particular fragments thereof which have been shown to encode non-variable regions of bacterial cell surface proteins in DNA constructs used in such genetic immunization experiments in animal models of infection with Streptococcus pneumoniae will be particularly useful for identifying protein epitopes able to provoke a prophylactic or therapeutic immune response. It is believed that this approach will allow for the subsequent preparation of monoclonal antibodies of particular value from the requisite organ of the animal successfully resisting or clearing infection for the development of prophylactic agents or therapeutic treatments of bacterial infection, particularly Streptococcus pneumoniae infection, in mammals, particularly humans.

The polypeptide may be used as an antigen for vaccination of a host to produce specific antibodies which protect against invasion of bacteria, for example by blocking adherence of bacteria to damaged tissue. Examples of tissue damage include wounds in skin or connective tissue caused, e.g., by mechanical, chemical or thermal damage or by implantation of indwelling devices, or wounds in the mucous membranes, such as the mouth, mammary glands, urethra or vagina.

The disclosure also includes a vaccine formulation which comprises an immunogenic recombinant protein of the disclosure together with a suitable carrier. Since the protein may be broken down in the stomach, it is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Results of cancer immunotherapy can range widely, including activation of anti-tumor immunity, negligible clinical effect, and hyperprogression of tumor growth. Along with considerable and significant efficacy, most cancers and patients do not response to current immunotherapy, and in a minority of patients cancer hyperprogression and rapid patient demise has been associated with immunotherapy, and has also been reported after chemotherapy or prior irradiation. (www.cancernetwork.com/lung-cancer/hyperprogression-nsclc-patients-pd-1pd-l1-inhibitors). Noting the low incidence of complete response to most metastatic cancer, Coventry and Ashdown propose that “. . . inflammatory and immune responses appear intricately associated with, if not causative of, complete responses induced by divergent forms of cancer therapy. Curiously, whether by chemotherapy, radiation, surgery, or other means, therapy-induced cell injury results, leaving inflammation and immune system stimulation as a final common denominator across all of these mechanisms of cancer therapy.”

(Coventry B J, Ashdown M L. Complete clinical responses to cancer therapy caused by multiple divergent approaches: a repeating theme lost in translation. Cancer Manag Res. 2012; 4:137-49; reviewed in Cowan F M. Connecting science past and future. J Chin Med Assoc 2017; 80:1-2).

Hyperprogression is more prevalent in females, perhaps associated with tolerance immunity needed for reproduction. Circulating cancer hyper-mutated DNA is predictive of both efficacy of immunotherapy and hyperprogression. (Khagi Y, Goodman A M, Daniels G A, Patel S P, Sacco A G, Randall J M, Bazhenova L A, Kurzrock R. Hyper-Mutated Circulating Tumor DNA: Correlation with Response to Checkpoint Inhibitor-Based Immunotherapy. Clin Cancer Res. 2017 Oct. 1; 23(19): 5729-5736. doi:10.1158/1078-0432.CCR-17-1439; Cabel L, Proudhon C, Romano E, Girard N, Lantz O, Stern M-H, Pierga J-Y, Bidard F C. Clinical potential of circulating tumour DNA in patients receiving anticancer immunotherapy. Nature Reviews Clinical Oncology 2018. volume 15, pages 639-650.) This may be reflective of cancer-DNA antibody IC FcR mediated tolerance and rejection immunity.

Antigen determinates and size (hapten) can also influence immunogenicity. (Genocea Unveils Possible Mechanism for Tumor Resistance to Immunotherapy at Society for Immunotherapy of Cancer's (SITC) 33rd Annual Meeting. Nov. 6, 2018 08:00 ET Source: Genocea Biosciences, Inc. ATLAS™-identified “inhibitory” neoantigens promote tumor growth in mouse model.

(www.globenewswire.com/news-release/2018/11/06/1645941/0/en/Genocea-Unveils-Possible-Mechanism-for-Tumor-Resistance-to-Immunotherapy-at-Society-for-Immunotherapy-of-Cancer-s-SITC-33rd-Annual-Meeting.html).

Cancer hyperprogression has been specifically associated with FcR immunity (Lo Russo G, Moro M, Sommariva M, Cancila V, Boeri M, Centonze G, Ferro S, Ganzinelli M, Gasparini P, Huber V, Milione M, Porcu L, Proto C, Pruneri G, Signorelli D, Sangaletti S, Sfondrini L, Storti C, Tassi E, Bardelli A, Marsoni S, Torri V, Tripodo C, Colombo M P, Anichini A, Rivoltini L, Balsari A, Sozzi G, Garassino M. Antibody-Fc/FcR interaction on macrophages as a mechanism for hyperprogressive disease in non-small cell lung cancer subsequent to PD-1/PD-L1 blockade. Clin Cancer Res 2019; 25:989-999). Commenting on this work, Knorr and Ravetch state that the mechanisms of tumor HP “remain ill-defined” with “a possible role for Fc receptors,” and they ask “how do we explain the same rate of HP clinically in NSCLC patients treated with PD-1/PD-L1 antibodies as they have major differences in FcR binding?” (Knorr D, Ravetch J V. Immunotherapy and hyperprogression: unwanted outcomes, unclear mechanism. Clin Cancer Res 2019; 25; 904-906). In 1968 Batchelor reviewed studies of antibody-dependent inhibition and “enhancement” of tumor growth and warned that, “[a]s regards human tumors, the main concern will be to avoid the occurrence of enhancement” (Batchelor, J R. The Use of Enhancement in Studying Tumor Antigens. Cancer Res 1968; 28:1410-1414).

### IBF Reagent

IBF and particularly microbial IBF have already demonstrated actions on FcR checkpoints and other FcR mediated immunity to include antibody dependent cellular cytotoxicity (ADCC), delayed hypersensitivity, when delayed hypersensitivity was in the lexicon a synonym for T cell immunity, and implicated in anti-cancer actions in humans and animals (Reviewed, Cowan, F. M. A Method for Treating Cellular Fc Receptor Mediated Immune Disorders. U.S. Pat. No. 5,189,014, Feb. 23, 1993 (original application filled 1979). IBF can further as predicted by Cowan 1979 can act as adjuvant of antigen presentation, further defined by (Léonetti M¹, Galon J, Thai R, Sautès-Fridman C, Moine G, Menez A. J Exp Med. 1999 Apr. 19; 189(8):1217-28). Presentation of antigen in immune complexes is boosted by soluble bacterial immunoglobulin binding proteins.] The disclosure envisions uses IBF-IC to accomplish IBF adjuvant, IC vaccine, FcR checkpoint and immune functions within a single molecular complex, greatly exceeding IBF therapy (Cowan, F. M. A Method for Treating Cellular Fc Receptor Mediated Immune Disorders. U.S. Pat. No. 5,189,014, Feb. 23, 1993 (original application filled 1979) by combining adjuvant, vaccine and checkpoint pharmacology in a single molecular complex that will not disassociate in sera. Said IBF-IC by using, for example, a defined caner antigen or the patient's own immune complex that contain the patient's own cancer specific antigens and/or cancer specific neoantigens that their immune response has already identified and targeted; yielding further amplification by the IBF adjuvant of immunity specific to a patient's cancer. The claimed conception of the pioneer patent and this new composition of matter and method of treatment acknowledges and cites others that saw parts but not the total disclosures of IBF immunotherapy or combination adjuvant IBF-IC vaccine checkpoint drugs. The prior art has not previously proposed or envisioned such a broad spectrum application of immunotherapy in a single molecular complex.

Presentation of antigen in immune complexes is boosted by soluble bacterial immunoglobulin binding proteins]. The disclosure provides for IBF-IC to accomplish, for example, IBF adjuvant, IC vaccine, FcR checkpoint and immune functions within a single molecular complex, greatly exceeding IBF therapy by combining adjuvant, vaccine and checkpoint pharmacology in a single molecule. Said IBF-IC, for example, by using a defined caner antigen or the patient's own immune complex that contain the patient's own cancer specific antigens and/or cancer specific neoantigens that their immune response has already identified and targeted a response toward; yields further amplification by the IBF adjuvant of immunity specific to a patient's cancer.

The Fc receptors, members of the immunoglobulin gene superfamily of proteins, are surface glycoproteins that can bind the Fc portion of immunoglobulin molecules. Each member of the family recognizes immunoglobulins of one or more isotypes through a recognition domain on the A-chain of the Fc receptor. Fc receptors are defined by their specificity for immunoglobulin subtypes. Fc receptors for IgG are referred to as FcγR, for IgE as FcR, and for IgA as FcαR. Different accessory cells bear Fc receptors for antibodies of different isotype, and the isotype of the antibody determines which accessory cells will be engaged in a given response (reviewed by Ravetch J. V. et al. (1991) “Fc Receptors,” Annu. Rev. Immunol. 9: 457-92; Gerber et al. (2001) “Stimulatory And Inhibitory Signals Originating From The Macrophage Fcgamma Receptors,” Microbes and Infection, 3: 131-139; Billadeau et al. (2002), “ITAMs Versus ITIMs: Striking A Balance During Cell Regulation,” The Journal of Clinical Investigation, 2(109): 161-168; Ravetch et al. (2000) “Immune Inhibitory Receptors,” Science, 290: 84-89; Ravetch et al., (2001) “IgG Fc Receptors,” Annu. Rev. Immunol. 19:275-290; Ravetch (1994) “Fc Receptors: RuborRedux,” Cell, 78(4): 553-560). The different Fc receptors, the cells that express them, and their isotype specificity is summarized in Table 1 (adapted from IMMUNOBIOLOGY: THE IMMUNE SYSTEM IN HEALTH AND DISEASE, 4th ed. 1999, Elsevier Science Ltd/Garland Publishing, New York).

FcR immune complex interaction is proposed as the key antigen specific immune regulatory “check point” that could influence all other immunotherapy to include antigen nonspecific check point regulators and inhibitors, a gateway to efficacy for antigen specific “focused cancer immunotherapy” (Cowan, F. M. A Method for Treating Cellular Fc Receptor Mediated Immune Disorders. U.S. Pat. No. 5,189,014, Feb. 23, 1993 (original application filled 1979), (Yu-mei Wen, Libing Mu, Yan Shi. Immunoregulatory functions of immune complexes in vaccine and therapy. EMBO Molecular Medicine. Published online: Aug. 29, 2016), (Mark J Smyth, Shin Foong Ngiow, Michele W L Teng. Using FcR to suppress cancer. Targeting regulatory T cells in tumor immunotherapy. Immunology and Cell Biology (2014) 92, 473-474).

Immune complex analysis reveals the specific and frequent presence of immune complex antigens in lung cancer patients (Zengguang Xu, Fengying Wu, Chunhong Wang, Xiyu Liu, Baoli Kang, Shan Shan, Xia Gu, Kailing Wang, Tao Ren. The stimulatory activity of plasma in patients with advanced non-small cell lung cancer requires TLR-stimulating nucleic acid immunoglobulin complexes and discriminates responsiveness to chemotherapy. Xu et al. Cancer Cell International 2014, 14:80 www.cancerci.com/content/14/1/80).

Ligand-receptor activation can be prevented either by blocking said receptor with an anti-receptor monoclonal antibody or soluble receptors bind the ligand and preventing it from engaging its receptor or by creating a lattice of aggregate antibody or IC greatly augment or enhance FcR binding and immunity such as antigen presentation. FcR are cell surface and soluble receptor IBF that are at the crossroads in coordination of innate to include the complement system, and adaptive antibody humoral and cellular immune regulation to include regulatory B and T cells. FcR also mediate effector function such as antibody dependent cellular cytotoxicity (ADCC) and phagocytosis. Fc antibody FcR ligand receptor interactions are in the new lexicon a central major “check point” for ligand-receptor immune regulation. Further FcR immunotherapy mediated inhibition or augmentation stated in the published science literature and patent prior art for decades are check point regulators or check point inhibitors. Check points are a new lexicon not a new idea.

The concept of use of receptors as drugs which are check point regulators is not much mentioned in immunotherapy histories, although this concept is used by FDA approved TNF monoclonal antibody and SPA-FcR (Protalex). The concept of use of check point regulator receptors drugs to TNF receptor like monoclonal antibodies (1980s) and FcR (1970s) is not new technology. This and several other more recent check point regulators that are check point blockers are FDA approved.

By strict definition binding the Fc portion of an antibody (Y lower part of antibody) to an FcR to block negative feedback tolerance is by definition a check point blocker (N. R. StC. Sinclair, “Why so many coinhibitory receptors?,” Scandinavian Journal of Immunology, 50(1): 10-3 Jul. 1999). FcR drugs such as SPA serve the same function as a check point regulators or blockers. FcR do not bind to FcR directly but perform the same function by preventing inhibitor immune complex Fc antibody from binding to FcR. FcR drugs as stated in our pioneer patent (Cowan, F. M. A Method for Treating Cellular Fc Receptor Mediated Immune Disorders. U.S. Pat. No. 5,189,014, Feb. 23, 1993 (original application filled 1979) can both block or enhance FcR mediated immunity whether this immunity is either positive or negative. If an FcR drug block a negative tolerance response this yield a minus a minus equals a positive (− −=+) or enhanced immune response that can break tolerance exactly as claimed for check point blockers. Positive or negative receptor ligand interactions are long known foundations of immune response that have previously yielded FDA approved drugs. Fc receptors are the “crossroad” of immunity that influence tolerance and rejection, and have long been proposed, tested, published and patented as immunotherapy. Immunity/inflammation can produce tolerance or rejection and this is so for cancer cells and other diseases, injuries, insults and conditions. All therapies for systemic cancer peripherally affect immunity and this effects efficacy and outcome (Coventry). Although the primary examples deal with breaking tolerance to cancer antigens this is exemplary not limiting. FcR immunity is constant for all antigens and can enhance immunity or tolerance. It is particular advantage of this disclosure that cancer protocols such as chemotherapy, radiation, surgery and other immunotherapy can give synergistic efficacy. Further, anti-inflammatory drugs, compounds and phytochemicals and many drugs with other primary actions with secondary anti-inflammatory actions are singularly and synergistically relevant to efficacy for cancer and other diseases, injuries, insults and conditions. Such drugs can include antineoplastic drugs such as statins or heparin and phytochemicals such as curcumin or turmeric.

The disclosure provides for vaccine compositions comprising, for example, IBF-IC, such as wherein the IBF reagent is an Fc reagent. The disclosure further provides for vaccine compositions comprising IBF-IC, such as wherein the IBF reagent is an Fc reagent selected from the group consisting of a microbial Fc receptor mimic, a microbial Fc receptor mimic comprising both Fab and FcR activities, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized to yield 4 or 5 FcR-Fab reagent binding units that can be further polymerized to form 10 to 100 FcR-Fab reagent, an FcR fragmented to give less than 4 to include monovalent single (added because of difference in action MSPA) of FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof.

### IC Reagent

Antigen antibody immune complex (IC) engaging FcR is a cornerstone of positive and negative immune regulation to include antigen specific immunity. IC are present in the sera of cancer patients, and can sometimes act as blocking factors to FcR mediated checkpoints, inhibiting cellular immunity to cancer neoantigens (Hellström K E, Hellström I, Snyder H W Jr, Balint J P, Jones F R. Blocking (suppressor) factors, immune complexes, and extracorporeal immunoadsorption in tumor immunity. Contemp Top Immunobiol. 1985; 15:213-38) One advantage of the present disclosure is instead of studying and searching for a myriad of neoantigens in general patient populations, the patient's own sera, for example in the case of a cancer patient, contains IC bound to their cancer neoantigens, the antigens most pertinent to the individual patient's cancer can be used. Further antigens or immune complex common for cancer can also be used as antigenic common denominators of cancer. It is a particular advantage that such cancer IC can be used as both a vaccine to enhance immunity, especially anticancer T cell immunity, and also as a biomarker for patients that are subjects for successful IBF-IC therapy. Only some perhaps a minority of patients will have circulation cancer neoantigens IC, demonstrated immunity to their cancer. This minority of IC positive patients may well represent a subpopulation of patients that respond to immunotherapy checkpoint drugs or immunological collateral actions of the triad of surgery, radiation and surgery indicatory of a small group to cancer patients who have complete response to cure their disease (Brendon J Coventry, Martin L Ashdown. Complete clinical responses to cancer therapy caused by multiple divergent approaches: a repeating theme lost in translation. Cancer Management and Research 2012:4 137-1490). On the reverse, IC are a double edged sword that can dependent on concentration or antibody isotype can cause feedback inhibit immunity and enhance caner growth, causing hyperprogression of cancer in some checkpoint drug treated patients. FcR containing cells are a biomarker for checkpoint efficacy and likewise IC may be biomarkers of immunotherapy influence on either positive cancer rejection or negative cancer hyper-progression immunity.

The disclosure provides IC from individual patients; In addition, the disclosure further provides IC constructed of known antigens that cause IC disease can be used or even just an isolated antigen bound to IBF. It is to be noted that such molecule, either IC of IBF can be isolated from sera standard culture procedures, or modified or manufactured by known means to improve valance, number of binding sites, affinity of binding, and can further be made by or synthesized by amino acid sequence and synthesis and cloning technology for product. Therefore, many equivalent forms of the disclosure can be made with minimal experimentation. In the disclosure's basic form, IC from cancer sera can be easily isolated and combined and fixed to IBF to form IBF-IC. IBF-IC can be added to an isolated cell population, and if desired such populations further incubated, cultivated and expanded, before infusion or just direct infusion of IBF-IC without ex vivo incubation. Further, IBF can just be administered or a hybrid system of isolated sera contain IC and cell populations can be incubated with free IBF to provide low tech and low cost version for developing nations that due to high costs are currently less served by immunotherapy (Cowan, F. M. A Method for Treating Cellular Fc Receptor Mediated Immune Disorders. U.S. Pat. No. 5,189,014, Feb. 23, 1993 (original application filled 1979), David S. Terman. PROTEIN A AND STAPHYLOCOCCAL PRODUCTS IN NEOPLASTIC DISEASE. CRC Critical Reviews in Ontology˜Hematology Volume 4, Issue 2 103-124, 1985).

It is well established that IC are present in circulation in sera and on immune cells. IC can contribute to, for example, pathological tolerance, immunity or autoimmunity or homeostasis to include cancer antigen and neoantigens containing IC. Antigen ligand antibody receptor for said ligand interaction is the primary lock and key mechanism for Immunity. The further engagement of FcR to Fc antibody renders FcR a central regulator, effector and checkpoint of immunity. IC vaccines have been utilized and can include attached adjuvant such as tetanus toxin; however, IBF coupled to IC as adjutants to treat IC diseases have not been disclosed. Fc reagent IBF can be soluble FcR, microbial proteins or be selected, constructed and synthesized by various means and tailor with specific binding affinity to particular antibody/immunoglobulin classes (IgG, IgA) and their isotypes (IgG1, IgG3). Microbial IBF have a particular advantage in that they can also bind Fab antibody constant region as Fab reagents. Collective FcR-Fab IBF reagents further bound to IC can more efficiently deliver IC to antigen presenting (APC) cells with greater immunity or tolerance efficacy. IC can be bound to IBF by a variety of chemical or synthesis means. Preferred best mode binders that are exemplary not limiting can include IBF-biotin further binding avidin, streptavidin, or “NeutrAvidin” bound to IC. One example of such an BF-biotin molecule is commercially available SPA-biotin.

The disclosure provides for vaccine compositions comprising IBF-IC, such as wherein the IC reagent is selected from the group consisting of synthetic, genetically engineered, monovalent, multivalent, isolated from a patient, and combinations thereof.

### Linker

The disclosure provides for vaccine compositions comprising IBF-IC, such as wherein the IBF reagent and IC reagent will not dissociate upon administration to a patient. The disclosure provides for vaccine compositions comprising IBF-IC, such as wherein the binding between the IBF reagent and IC reagent is selected from the group consisting of van der Waals forces, hydrogen bonds, ionic bonds, hydrophobic interactions, and combinations thereof. The disclosure provides for vaccine compositions comprising IBF-IC, such as wherein the IBF reagent and IC reagent are covalently bound. The disclosure provides for vaccine compositions comprising IBF-IC, such as

wherein the IBF reagent and IC reagent are covalently bound by a linker. The disclosure provides for vaccine compositions comprising IBF-IC, such as wherein the IBF reagent and IC reagent are covalently bound by a linker which is selected from the group consisting of a peptide, avidin, streptavidin, NeutrAvidin, biotin, and combinations thereof. The disclosure provides for vaccine compositions such as wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin. The disclosure provides for vaccine compositions wherein IBF reagent is covalently bound to IC reagent. The disclosure provides for vaccine compositions wherein IBF reagent is covalently bound to IC reagent by a peptide, by a non-peptide linker, or combinations thereof.

Methods for peptide crosslinking to form a covalent linkage between protein chains are well known by those of skill in the art, and include chemical, enzymatic, and photo-crosslinking methods. Such known methods include methods for using chemical linking agents to covalently crosslink antibody to other proteins, including methods used to crosslink antibody Fc region to FcR IBF. (Acchione, Kwon H, Jochheim C M, Atkins W M. Impact of linker and conjugation chemistry on antigen binding, Fc receptor binding and thermal stability of model antibody-drug conjugates, MAbs. 2012 May-June; 4(3):362-72; Sousa M M, Steen K W, Hagen L, Slupphaug G. Antibody cross-linking and target elution protocols used for immunoprecipitation significantly modulate signal-to noise ratio in downstream 2D-PAGE analysis. Proteome Sci. 2011 Aug. 4; 9:45). Methods for safe preparation of crosslinked protein structures of use as therapeutic drugs are also well known by those of skill in the art. Karimi M, Bahrami S, Ravari S B, Zangabad P S, Mirshekari H, Bozorgomid M, Shahreza S, Sori M, Hamblin M R. Albumin nanostructures as advanced drug delivery systems. Expert Opin Drug Deliv. 2016 November; 13(11):1609-1623.

The IBF-IC vaccine may, for example, be a fusion protein, e.g., a fusion protein expressed from a recombinant DNA which encodes the IBF-IC vaccine and at least one protein of interest or a fusion protein formed by chemical synthesis. A conjugate encompasses chemical conjugates (covalently or non-covalently bound), fusion proteins and the like. For instance, the fusion protein may comprise a IBF-IC vaccine and an enzyme of interest, e.g., luciferase, RNasin or RNase, and/or a channel protein, a receptor, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, a transcription factor, a transporter protein and/or a targeting sequence, e.g., a myristilation sequence, a mitochondrial localization sequence, or a nuclear localization sequence, that directs the IBF-IC vaccine, for example, a fusion protein, to a particular location. The IBF-IC vaccine protein of interest may be fused at the N-terminus or the C-terminus. In one embodiment, the IBF-IC vaccine fusion protein comprises Optionally, the proteins in the fusion are separated by a linker connector sequence, e.g., one having at least 1 or 2 amino acid residues, such as one having 13 to 17 amino acid residues. The presence of a linker connector sequence in a fusion protein of the disclosure does not substantially alter the function of either protein in the fusion relative to the function of each individual protein.

As mentioned above, the IBF-IC peptides of the disclosure may comprise at least one IBF domain and at least one IC domain, which may be linked to each other via a non-naturally occurring intervening amino acid sequence. According to the disclosure, said non-naturally occurring intervening amino acid sequence act as a hinge region between said domains, allowing them to move independently of one another while maintaining the three-dimensional shape of the individual domains. In that sense, a preferred non-naturally occurring intervening amino acid sequence according to the disclosure would be a hinge region characterized by a structural softness that enables this motion. In a particular embodiment, said non-naturally occurring intervening sequence is a non-naturally occurring flexible linker. In a preferred embodiment, said flexible linker is a flexible peptide linker with a length of 20 or less amino acids. In a more preferred embodiment, the peptide linker comprises 2 or more amino acids selected from the group consisting of glycine, serine, alanine and threonine. In a preferred embodiment of the disclosure, said flexible linker is a poly-glycine linker.

The linker peptide may comprise at least one, or two amino acids, such as at least three amino acids, for example at least five amino acids, such as at least ten amino acids, for example at least 15 amino acids, such as at least 20 amino acids, for example at least 30 amino acids, such as at least 40 amino acids, for example at least 50 amino acids, such as at least 60 amino acids, for example at least 70 amino acids, such as at least 80 amino acids, such as at least 90 amino acids such as approximately 100 amino acids.

In a particular embodiment of the disclosure, the IBF and/or IC may comprise Avidin: As defined herein, “avidin” includes avidin, streptavidin, neutravidin, and derivatives and analogs thereof that are capable of high affinity, multivalent or univalent binding of biotin. In a particular embodiment of the disclosure, the IBF and/or IC may comprise biotin. In a particular embodiment of the disclosure, the IBF peptide comprises avidin, streptavidin, neutravidin, and derivatives and analogs thereof while the IC peptide comprises biotin. In a particular embodiment of the disclosure, the IC peptide comprises avidin, streptavidin, neutravidin, and derivatives and analogs thereof while the IBF peptide comprises biotin.

In a particular embodiment of the disclosure, the polypeptides of the disclosure may comprise the amino acid sequence of a tag. Said tag includes but is not limited to: polyhistidine tags (His-tags) (for example H6 and H10, etc.) or other tags for use in IMAC systems, for example, Ni2+ affinity columns, etc., GST fusions, MBP fusions, streptavidine-tags, the BSP biotinylation target sequence of the bacterial enzyme BRA and tag epitopes that are directed by antibodies (for example c-myc tags, FLAG-tags, among others). As will be observed by a person skilled in the art, said tag peptide can be used for purification, inspection, selection and/or visualization of the fusion protein of the disclosure. In a particular embodiment of the disclosure, said tag is a detection tag and/or a purification tag. When the polypeptide of the disclosure comprises an N-terminal His6× tag and a C-terminal epitope, these can be recognised by specific antibodies allowing immunoprecipitation, immunodetection (ELISA and Western-blot) and sub-cellular distribution by indirect immunofluorescence. Hence, in a preferred embodiment of the disclosure, said detection tag is the Sv5 epitope tag and said purification tag is a polyhistidine tag.

### Conditions

The disclosure provides for methods of treating conditions such as immune complex diseases or disorders by administration to a patient of vaccine compositions comprising IBF-IC, such as wherein the patient has a condition selected from the group consisting of cancer, abnormal immunity to antigen, abnormal FcR mediated immunity to antigen, and combinations thereof. The disclosure provides for methods of treating conditions by administration to a patient of vaccine compositions comprising IBF-IC, such as wherein the vaccine prevents and/or treats an immune complex disease or disorder.

The disclosure provides for methods of treating conditions by administration to a patient of vaccine compositions comprising IBF-IC, such as wherein the immune complex disease or disorder is selected from the group consisting of cancer, abnormal immunity to antigen, abnormal FcR mediated immunity to antigen, and combinations thereof.

The disclosure provides for methods of treating conditions by administration to a patient of vaccine compositions comprising IBF-IC, such as wherein the vaccine treats and/or prevents a pathological infection.

The disclosure provides for methods of treating conditions by administration to a patient of vaccine compositions comprising IBF-IC, such as wherein the pathological infection is selected from the group consisting of bacterial infection, viral infection, fungal infection, parasitic infection, and combinations thereof.

The disclosure provides for methods of treating conditions by administration to a patient of compositions comprising nucleic acids encoding IBF-IC, such as wherein the immune complex disease or disorder is selected from the group consisting of cancer, abnormal immunity to antigen, abnormal FcR mediated immunity to antigen, and combinations thereof.

The disclosure provides for methods of treating conditions by administration to a patient of vaccine compositions comprising nucleic acids encoding IBF-IC, such as wherein the vaccine treats and/or prevents a pathological infection.

The disclosure provides for methods of treating conditions by administration to a patient of vaccine compositions comprising nucleic acids encoding IBF-IC, such as wherein the pathological infection is selected from the group consisting of bacterial infection, viral infection, fungal infection, parasitic infection, and combinations thereof.

The disclosure provides for methods of treating conditions by administration to a patient of compositions comprising nucleic acids encoding IBF and/or IC, such as wherein the immune complex disease or disorder is selected from the group consisting of cancer, abnormal immunity to antigen, abnormal FcR mediated immunity to antigen, and combinations thereof.

The disclosure provides for methods of treating conditions by administration to a patient of vaccine compositions comprising nucleic acids encoding IBF and/or IC, such as wherein the vaccine treats and/or prevents a pathological infection.

The disclosure provides for methods of treating conditions by administration to a patient of vaccine compositions comprising nucleic acids encoding IBF and/or IC, such as wherein the pathological infection is selected from the group consisting of bacterial infection, viral infection, fungal infection, parasitic infection, and combinations thereof.

Examples of inflammatory and/or autoimmune disorders include, but are not limited to, primary immune thrombocytopenia (ITP), systemic lupus erythematosus (SLE), rheumatoid arthritis (RA), autoimmune haemolytic anaemia (AIHA), diabetes, Pemphigus vulgaris, Hashimoto's thyroiditis, autoimmune inner ear disease myasthenia gravis, pernicious anemia, Addison's disease, dermatomyositis, Sjögren's syndrome, dermatomyositis, multiple sclerosis, Reiter's syndrome, Graves disease, autoimmune hepatitis, familial adenomatous polyposis and ulcerative colitis.

According to a specific embodiment of the present disclosure, there is provided a method of immunotherapy for treatment or alleviating the symptoms of a disease related to the immune response, that is a neoplastic disease e.g. cancer/malignancy, in a patient suffering from such a disease, wherein abnormal Fc receptor mediated immunity to the invading antigen contributes to the pathology or symptoms of the disease, which method comprises administering to said patient an effective dose of an IBF-IC vaccine which reacts directly or indirectly with the Fc portion of antibody or immune complex enhancing the body's own endogenous cellular Fc receptor mediated immune response and overcoming the abnormal Fc receptor mediated immunity to the invading neoplastic antigen.

According to a specific embodiment of the present disclosure, there is provided a method of immunotherapy for treating carcinoma which comprises enhancing the body's own endogenous cellular Fc receptor mediated immune response towards the invading antigen by administering an effective amount of an IBF-IC vaccine. The treatment of a cancer patient will be given as exemplary not limiting. First the cancer patient with immune complex disease will be identified. The type of immune complex, and antigens bound, amount and properties of immune complex can vary for different cancer, different stages of cancer and between individual patients and between different phases of and responses to therapy.

According to a specific embodiment of the present disclosure, there is provided a method of immunotherapy for treating adenocarcinoma which comprises enhancing the body's own endogenous cellular Fc receptor mediated immune response towards the invading antigen by administering an effective amount of an IBF-IC vaccine.

According to a specific embodiment of the present disclosure, there is provided a method of immunotherapy for treating mammary adenocarcinoma which comprises enhancing the body's own endogenous cellular Fc receptor mediated immune response towards the invading antigen by administering an effective amount of an IBF-IC vaccine.

According to another embodiment of the present disclosure, there is provided a method of immunotherapy for treating or alleviating the symptoms of a disease related to the immune response, that is, transplant rejection in a patient undergoing an organ or tissue transplant, wherein abnormal Fc receptor mediated immunity to the transplantation antigen contributes to the pathology or symptoms of the disease, which comprises administering an effective immuno-suppressive amount of an IBF-IC vaccine which is capable of binding antibodies or immune complexes formed in response to the foreign graft providing an immuno-suppressive effect on the cellular Fc-receptor mediated graft rejection and preventing the formation of sensitized lymphocytes.

According to another embodiment of the present disclosure, there is provided a method of immunotherapy for treating or alleviating the symptoms of a disease related to the immune response, that is, an autoimmune diseases wherein the body has developed an immunity against some of its own tissue proteins and abnormal Fc receptor mediated immunity to the disease antigen contributes to the pathology or symptoms of the disease. Examples of such autoimmune diseases include multiple sclerosis, myasthenia gravis, pernicious anemia, Addison's disease, Goodpasture's disease, systemic lupus erythematosus, rheumatoid arthritis. Treatment and/or prophylaxis of these autoimmune diseases according to the present disclosure comprises administering to a patient in need of such treatment an immuno-suppressive effective amount of an IBF-IC vaccine, which is capable of binding antibodies or immune complexes produced as part of the bodies autoimmune response and preventing the formation of sensitized lymphocytes by antibodies or immune complexes.

According to another embodiment, there is provided a method for improving the compatibility of drugs which can cause allergic side reactions due to complement interaction with a cellular Fc receptor mediated immune response e.g. anaphylaxis. In the case of a combination of an exogenous multivalent Fc receptor with an exogenous monovalent Fc receptor, the ratio between monovalent and multivalent Fc receptors is in the range of from about 1:1 to about 10:1.

According to another embodiment of the present disclosure, there is provided a method of immunotherapy for reducing the severity of diseases which are caused by complement interaction with a cellular Fc receptor mediated immune response, such as glomerulonephritis or anaphylaxis which comprises administering an effective amount of an exogenous monovalent Fc receptor, which is sufficient to interfere with the attachment of antibodies on lymphocytes in a pattern which enhances complement interaction.

Neoplasia, Cancer, Tumors, Proliferative Diseases, Malignancies and their Metastases

The disclosure provides a method for treating neoplasia in a patient in need of such treatment, comprising: administering to the patient therapeutically effective amounts of a IBF-IC vaccine as exemplified herein, in combination with, for example, a neoplasia treating agent. The term “neoplasia” as used herein refers also to tumors, proliferative diseases, malignancies and their metastases. Examples for cancer diseases are adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, non-small cell lung cancer (NSCLC), breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, squamous cell carcinoma of the head and neck (SCCHN), prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

The neoplasia may be, for example, selected for the group consisting of hepatocellular carcinoma, esophageal squamous cell carcinoma, breast cancer, pancreatic cancer, squamous cell cancer or adenocarcinoma of the head and neck, colorectal cancer, renal cancer, brain cancer, prostate cancer, small and non-small cell lung cancer, bladder cancer, bone or joint cancer, uterine cancer, cervical cancer, multiple myeloma, hematopoietic malignancies, lymphoma, Hodgkin's disease, non-Hodgkin's lymphoma, skin cancer, melanoma, squamous cell carcinoma, leukemia, lung cancer, ovarian cancer, stomach cancer, Kaposi's sarcoma, laryngeal cancer, endocrine carcinomas, cancer of the thyroid gland, cancer of the parathyroid gland, cancer of the pituitary gland, cancer of the adrenal gland, and combinations thereof.

### Bacterial Pathogens

The compositions and methods of the disclosure may be used to treat and/or prevent a pathological infection, including, for example, bacterial infection. There are hundreds of bacterial pathogens in both the Gram-positive and Gram-negative families that cause significant illness and mortality around the word, despite decades of effort developing antibiotic agents. Antibiotic resistance is a growing problem in bacterial disease. Bacterial pathogens may be prevented and/or treated by the compositions and methods of the disclosure.

One of the bacterial diseases with highest disease burden is tuberculosis, caused by the bacterium Mycobacterium tuberculosis, which kills about 2 million people a year, mostly in sub-Saharan Africa. Pathogenic bacteria contribute to other globally important diseases, such as pneumonia, which can be caused by bacteria such as Streptococcus and Pseudomonas, and food borne illnesses, which can be caused by bacteria such as Shigella, Campylobacter, and Salmonella. Pathogenic bacteria also cause infections such as tetanus, typhoid fever, diphtheria, syphilis, and leprosy.

Conditionally pathogenic bacteria are only pathogenic under certain conditions, such as a wound facilitates entry of bacteria into the blood, or a decrease in immune function. For example, Staphylococcus or Streptococcus are also part of the normal human flora and usually exist on the skin or in the nose without causing disease, but can potentially cause skin infections, pneumonia, meningitis, and even overwhelming sepsis, a systemic inflammatory response producing shock, massive vasodilation and death. Some species of bacteria, such as Pseudomonas aeruginosa, Burkholderia cenocepacia, and Mycobacterium avium, are opportunistic pathogens and cause disease mainly in people suffering from immunosuppression or cystic fibrosis.

Other bacteria invariably cause disease in humans, such as obligate intracellular parasites (e.g., Chlamydophila, Ehrlichia, Rickettsia) that are capable of growing and reproducing only within the cells of other organisms. Still, infections with intracellular bacteria may be asymptomatic, such as during the incubation period. An example of intracellular bacteria is Rickettsia. One species of Rickettsia causes typhus, while another causes Rocky Mountain spotted fever. Chlamydia, another phylum of obligate intracellular parasites, contains species that can cause pneumonia or urinary tract infection and may be involved in coronary heart disease. Mycobacterium, Brucella, Francisella, Legionella, and Listeria can exist intracellular, though they are facultative (not obligate) intracellular parasites.

Gram-positive bacteria include Staphylococcus aureus; Staphylococcus epidermidis; Staphylococcus saprophyticus; Streptococcus pyogenes (Lancefield group A, beta-hemolytic); Streptococcus agalactiae (Lancefield group B, beta-hemolytic); Streptococcus Viridans group (most are alpha-hemolytic) including, for example, the mitus group (S. mitus, S. sanguis, S. parasanguis, S. gordonii, S. crista, S. infantis, S. oralis, S. peroris), the Salivarius group (S. salivarius, S. vestibularis, S. thermophilus), the Mutans group (S. mutans, S. sobrinus, S. criceti, S. rattus, S. downei, S. macacae), and the Anginosus group (S. anginosus, S. constellatus, S. intermedius); Streptococcus, e.g., S. bovis, S. equinus (Lancefield group D, alpha-hemolytic); Streptococcuspneumoniae (no Lancefield antigen; alpha-hemolytic); Peptostreptococcus and Peptococcus; Entercoccus faecalis; Enterococcus faeccium; Cornybacterium diphtheria; Bacillus anthracis; Bacillus cereus; Clostridium C. botulinum (more rarely, C. baratii and C. butyricum); Clostridium tetani; Clostridium perfringens; Clostridium difficile; Clostridium sordellii; Listeria monocytogenes; Actinomyces israelii; Nocardia asteroids; Streptomyces.

Gram-negative bacteria include Neisseria meningitides; Neisseria gonorrhoeae; Moraxella (subgenera Branhamella) catarrhalis; Kingella (most commonly kingae); Acinetobacter baumannii, Oligella ureolytica; Oligella urethralis; Escherichia coli; Shigella (S. dysenteriae, S. flexneri, S. boydii, S. sonnei); Salmonella non typhoidal, including S. enterica serotype enteritidis, S. enterica serotype typhimurium, S. enterica serotype Choleraesuis, S. bongori, Salmonella S. enterica serotype Typhi; Yersinia enterocolitica, Klebsiella pneumoniae; Proteus mirabilis; Enterobacter; Cronobacter (formerly called Enterobacter sakazakii); Serratia; Edwardsiella; Citrobacter; Hafnia; Providencia; Vibrio cholera; Vibrio parahemolyticus; Campylobacter; Helicobacter (formerly called Campylobacter) pylori, Pseudomonas aeruginosa; Burkholderia cepacia; Burkholderia mallei; Burkholderia pseudomallei; Stenotrophomonas maltophilia; Bacteroides fragilis, Bacteroides melaninogenicus; Fusobacterium; Haemophilus influenza; Haemophilus ducreyi; Gardnerella (formerly called Haemophilus) vaginalis; Bordetella pertussis; Legionella; Yersinia pestis; Francisella tularensis; Brucella B. melitensis (infects sheep/goats); B. abortus (abortions in cows); B. suis (pigs); B. canis (dogs); B. maris (marine animals); Pasteurella multocida; Streptobacillus moniliformis; Spirillum minus; Treponema pallidum; Treponema pallidum subspecies pertenue; Treponema pallidum subspecies endemicum; Treponema pallidum subspecies carateum; Borrelia burgdorferi; Borrelia; Leptospira; Chlamydia trachomatis; Chlamydia pneumonia; Chlamydia psittaci; Rickettsiae rickettsia; Rickettsiae akari; Rickettsiae prowazekii; Rickettsiae typhi; Rickettsiae tsutsugamushi; Rickettsiae parkeri; Rickettsiae africae; Rickettsia conorii; Rickettsia australis; Rickettsia siberica; Rickettsia japonica; Bartonella Quintana; Bartonella henselae; Bartonella bacilliformis; Coxiella burnetii; Ehrlichia; Anaplasma phagocytophilum; Neorickettsia; Orientia; Klebsiella granulomatis (formerly called Calymmatobacterium granulomatis); Capnocytophaga.

Other bacteria include Mycobacterium tuberculosis; Mycobacterium bovis; Mycobacterium leprae; Mycobacterium avium-intracellulare or avium complex (MAI or MAC); Mycobacterium ulcerans; Mycobacterium kansasii; Mycobacterium marinum; Mycobacterium scrofulaceum; Mycobacterium fortuitum; Mycobacterium chelonei; Mycobacterium abscessus; Mycoplasma pneumonia; Ureaplasma urealyticum.

### Viral Pathogens

The compositions and methods of the disclosure may be used to treat and/or prevent a pathological infection, including, for example, viral infection. Viruses include DNA and RNA viruses. These include respiratory viruses such as Adenoviruses, Avian influenza, Influenza virus type A, Influenza virus type B, Measles, Parainfluenza virus, Respiratory syncytial virus (RSV), Rhinoviruses, and SARS coronavirus, gastro-enteric viruses such as Coxsackie viruses, enteroviruses such as Poliovirus and Rotavirus, hepatitis viruses such as Hepatitis B virus, Hepatitis C virus, Bovine viral diarrhea virus (surrogate), herpes viruses such as Herpes simplex 1, Herpes simplex 2, Human cytomegalovirus, and Varicella zoster virus, retroviruses such as Human immunodeficiency virus 1 (HIV-1), and Human immunodeficiency virus 2 (HIV-2), as well as Dengue virus, Hantavirus, Hemorrhagic fever viruses, Lymphocytic choriomeningitis virus, Smallpox virus, Ebola virus, Rabies virus, West Nile virus (WNV) and Yellow fever virus. In certain embodiments as disclosed herein an IC-IBF vaccine as disclosed here has anti-viral activity, such as anti-coronavirus activity.

Examples of viruses which may be prevented and/or treated by the compositons and methods of the disclosure include Parvoviridae; Papovaviridae (Human papilloma virus (HPV); BK polyomavirus; JC polyomavirus); Adenoviridae (Adenovirus, types 40 and 41); Herpesviridae (simplex virus type 1 (HHV-1); Herpes simplex virus type 2 (HHV-2); Macacine herpesvirus 1; Varicella-zoster virus (VZV; HHV-3); Epstein-Barr virus (EBV; HHV-4); Cytomegalovirus (CMV; HHV-5); Human Herpesvirus 6 (HHV-6); HHV-7; Kaposi's sarcoma-associated herpesvirus (HHV-8); Hepadnaviridae (Hepatitis B virus); Poxviridae (Smallpox (Variola major); Alastrim (Variola minor); Vaccinia; Cowpox; Monkeypox; Goat pox, pseudocowpox virus, bovine papular stomatitis virus, tanapox, volepox and related pox viruses such as avipox, buffalopox, racoonpox, squirrelpox, etc.); Molluscum contagiosum; Picornaviridae (Polio virus; Coxsackie A virus; Coxsackie B; virus; Foot and mouth disease; ECHO virus; Hepatitis A virus; Rhinovirus); Astroviridae; Caliciviridae (Norwalk virus; Norovirus; Sapoviruses; Hepatitis E virus); Reoviridae (Rotavirus); Togaviridae (Alpha viruses; Western equine encephalitis (WEE) virus; Eastern equine encephalitis (EEE) virus; Venezuelan equine encephalitis (VEE) virus; Chikungunya virus; Rubivirus (rubella)); Flaviviridae (Yellow fever virus; Dengue virus; St. Louis encephalitis virus; Japanese encephalitis virus; Tick-borne encephalitis virus; Omsk hemorrhagic fever virus; Al Khumra virus; Kyasanur Forest disease virus; Louping ill virus; West Nile virus; Kunjin virus; Murray Valley fever virus; Powassan virus; Hepatitis C virus; Hepatitis G virus); Coronoviridae (Respiratory illness (cold); Severe Acute Respiratory Syndrom)-corona virus (SARS-CoV)); Bunyaviridae (California encephalitis virus; La Crosse virus; Rift Valley fever virus; Phleboviruses; Sandfly fever virus; Nairovirus; Hantavirus); Orthomyxoviridae (Influenza virus (types A, B & C); Paramyxoviridae (Parainfluenza virus; Respiratory syncytial virus (RSV); Hendra virus disease (formerly equine morbillivirus); Nipah virus encephalitis; Mumps Measles; Newcastle disease virus); Rhabdoviridae (Rabies virus); Filoviridae (Marburg virus (acute hemorrhagic fever); Ebola virus (acute hemorrhagic fever)); Arenaviridae (Lymphocytic choriomeningitis virus; Lassa fever virus; Lujo virus; Chapare virus; Junin virus; Machupo virus; Guanarito virus; Sabia virus); Retroviridae (Human Immunodeficiency virus (HIV) types I and II; Human T-cell leukemia virus (HLTV) type I; Human T-cell leukemia virus (HLTV) type II; Spumaviruses; Xenotropic murine leukemia virus-related (XMRV).

### Fungal Pathogens

The compositions and methods of the disclosure may be used to treat and/or prevent a pathological infection, including, for example, fungal infections. Pathogenic fungi are fungi that cause disease in humans or other organisms. The pathogenic fungi which may be prevented and/or treated by the compositions and methods of the disclosure include but are not limited to the following.

Candida species are important human pathogens that are best known for causing opportunist infections in immunocompromised hosts (e.g., transplant patients, AIDS sufferers, and cancer patients). Infections are difficult to treat and can be very serious. Aspergillus can and does cause disease in three major ways: through the production of mycotoxins; through induction of allergenic responses; and through localized or systemic infections. With the latter two categories, the immune status of the host is pivotal. The most common pathogenic species are Aspergillus fumigatus and Aspergillus flavus. Cryptococcus neoformans can cause a severe form of meningitis and meningo-encephalitis in patients with HIV infection and AIDS. The majority of Cryptococcus species lives in the soil and do not cause disease in humans. Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease in non-immunocompromised people. Histoplasma capsulatum can cause histoplasmosis in humans, dogs and cats. Pneumocystis jirovecii (or Pneumocystis carinii) can cause a form of pneumonia in people with weakened immune systems, such as premature children, the elderly, transplant patients and AIDS patients. Stachybotrys chartarum or “black mold” can cause respiratory damage and severe headaches. It frequently occurs in houses in regions that are chronically damp.

Examples include Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum.

### Nucleic Acids

One aspect of the present disclosure is polynucleotide sequences encoding IBF and/or IC sequences, derivatives thereof, and homologs thereof, the complement of these sequences, the RNA versions of both DNA strands and the information otherwise contained within the linear sequence of these polynucleotide sequences and fragments thereof. In the case of nucleic acid segments, sequences for use with the present disclosure are those that have greater than about 50 to 60% homology with any portion of the polynucleotide sequences described herein, sequences that have between about 61% and about 70%; sequences that have between about 71 and about 80%; or between about 81% and about 90%; or between 91% and about 99%; or which contain nucleotides that are identical, functionality equivalent, or functionally irrelevant, with respect to the nucleotides present in IBF and/or IC sequences are considered to be essentially similar. Also encompassed within the present disclosure are nucleic acids that encode polypeptides that are at least 40% identical or similar to IBF and/or IC amino acid sequences.

The disclosure also encompasses other nucleic acids or nucleic acid like molecules that are sufficient in any regard to mimic, substitute for, or interfere with the IBF and/or IC polynucleotide sequences, or fragments thereof. It will also be understood that the nucleic acid and amino acid sequences may include additional residues, such as additional 5′- or 3′-sequences, and yet still be essentially as set forth in one of the sequences disclosed herein, so long as the sequence meets the criteria set forth, including the maintenance of functionality, or for the purpose of engineering altered functionality with respect to IBF and/or IC.

Included within the disclosure are DNA or RNA segments including oligonucleotides, polynucleotides and fragments thereof, including DNA or RNA or nucleic acid-like sequences of genomic or synthetic origin, single or double stranded. The disclosure includes nucleic acid molecules, or nucleic acid-like molecules that are able to hybridize to the IBF and/or IC sequences, under stringent or under permissive hybridization conditions, or to the complement of said sequences.

The disclosure also includes oligonucleotide, or oligonucleotide-like sequences such as phosphorthioate, or peptide nucleic acid sequences that possess sufficient similarity with the sequences disclosed herein such that they are able to stably hybridize to the disclosed sequences, or their complements. Such sequences may be intended as antisense regulators of gene expression, or for the selective amplification or extension of adjoining sequences, for instance by PCR using a given annealing temperature, as would be determined by someone skilled in the art.

In addition to the sequences disclosed here, related sequences in other organisms, or homologs, will be readily identified by hybridization using the present sequences, with respect to IBF and/or IC sequences, and similar sequences. Thus, related genes, and related mRNA transcripts, can be identified by one skilled in the art. The disclosure thus encompasses methods for the use of the disclosed sequences in various screening procedures aimed at isolating such species. For instance, colony or plaque hybridization techniques can be performed using radiolabeled sequences as a probe to detect complementary sequences in genomic and cDNA libraries.

Hybridization conditions with respect to temperature, formamide and salt concentrations, in such studies are chosen by one skilled in the art and vary with respect to the organism from which sequences are being isolated, and the sequence similarity, or lack thereof, that is expected based on evolutionary distances. Similar techniques will apply to the isolation of the genomic sequences that encode IBF and/or IC, as well as those that encode related genes from organisms other than humans. Reference is particularly made to flanking regions, including upstream sequences that encode the core promoter and regulatory regions, as well as downstream regions, introns and intron/exon boundaries. Similar techniques will also apply to the identification of mutant alleles, polymorphisms, deletions, insertions, and so forth, in genomic and cDNA sequences. These may occur within the IBF and/or IC sequences themselves, or may occur in regulatory regions, introns, intron/exon boundaries, or may reflect various insertions, partial or whole gene deletions, or substitutions, any of which may affect biological activity of a gene and gene product. In the case of humans, the identification of interindividual genomic differences in the IBF and/or IC genes will be useful in diagnostic determinations.

Whole or partial sequences referred to above may also be identified and isolated using techniques that involve annealing of short oligonucleotides to complementary sequences, such as those as might be present in the genomic DNA of a particular organism, or in genomic or cDNA, including expression cDNA, libraries. Thus, PCR is used to obtain DNA sequences homologous to, and which lie between, two primers, usually between 15 to 30 nucleotides which have annealing temperatures typically between 60-80 degrees Celsius may be substantially purified. The choice of primer sequences, annealing conditions (temperature), number of amplification cycles, choice of polymerase, and so forth would be within the knowledge of one skilled in the art. Amplification assays will be generally applicable to the identification of sequences homologous to IBF and/or IC, to the identification of flanking genomic or cDNA sequences, to the identification of mutated alleles, and so forth, in a manner that lends itself to rapid diagnostics.

Variations in PCR technology are also relevant, such as reverse transcriptase mediated PCR, in which mRNA or total RNA is reverse transcribed typically with an oligo dT or gene specific primer prior to PCR amplification. Techniques are also available which utilize only one gene-specific primer, together with a linker or adapter primer as may be present in a vector or attached to the ends of the DNAs to be amplified. For instance, the Genome Walker (Clontech) technique allows the isolation of genomic DNA that flanks a given oligonucleotide primer. Techniques are also available in which altered oligonucleotides are employed to generate specific mutations, deletions, insertions, or fusions in the disclosed sequences, or fragments thereof, for instance site directed mutagenesis.

Naturally, it will be understood that this disclosure is not limited to the particular nucleic acid sequences presented herein. Recombinant vectors, including for example plasmids, phage, viruses, and other sequences, and isolated DNA or RNA segments may therefore variously include the IBF and/or IC gene sequences or their complements, and coding regions, as well as those that may bear selected alterations or modifications that nevertheless include IBF and/or IC segments or may encode biologically or experimentally relevant amino acid sequences. Such sequences may be created by the application of recombinant DNA technology, where changes are engineered based on the consideration of the nucleotides or amino acids being exchanged, deleted, inserted, fused, or otherwise modified.

Likewise, the current disclosure encompasses sequences that may be naturally present as extensions of, or insertions within, the sequences disclosed herein, including alternative or longer 5′ or 3′ mRNA sequences, or intronic and promoter genomic sequences, or allelic or polymorphic versions of a gene. Similarly, natural, artificial, or synthetic fusions of IBF and/or IC, and fragments thereof, with unrelated nucleic acids or amino acids such as those that encode epitope tags, binding proteins, marker proteins, and other amino acid sequences are included.

IBF and/or IC Proteins and Polypeptides

One aspect of the disclosure is the protein, polypeptide, oligopeptide, or amino acid sequences or fragments thereof, of IBF and/or IC. Sequences that have greater than about 40-50% homology with any portion of the amino acid sequences described herein, sequences that have between about 51% and about 60%; sequences that have between about 61% and about 70% sequences that have between about 70 and about 80%; or between about 81% and about 90%; or between 91% and about 99%; or those that contain amino acids that are identical, functionally equivalent, or functionally irrelevant, for instance those specified by conservative, evolutionarily conserved, and degenerate substitutions, with respect to IBF and/or IC amino acid sequences are included. Functions of the IBF and/or IC polypeptides of the instant disclosure include but are not limited to binding of FcR. The disclosure thus applies to IBF and/or IC polypeptide sequences, or fragments thereof, and nucleic acids which encode such polypeptides, such as those of other species. Reference is particularly, but not exclusively, made to the conserved regions of IBF and/or IC, in contrast to similarity throughout the entire length. The disclosure thus encompasses amino acid sequences, or amino acid-like molecules, that are sufficient in any regard to mimic, substitute for, or interfere with the IBF and/or IC amino acid sequences, or fragments thereof.

The disclosure encompasses IBF and/or IC amino acid sequences that have been altered in any form, either through the use of recombinant engineering, or through post-translational or chemical modifications, including those that may be produced by natural, biological, artificial, or chemical methods. Naturally, it will be understood that this disclosure is not limited to the particular amino acid sequences presented herein. Altered amino acid sequences include those which have been created by the application of recombinant technology such that specific residues, regions, or domains have been altered, and which may be functionally identical, or which may possess unique biological or experimental properties with regards to function or interactions with natural and artificial ligands.

For instance such modifications may confer longer or shorter half-life, reduced or increased sensitivity to ligands that modify function, ability to detect or purify polypeptides, solubility, and so forth. Alternatively, such sequences may be shorter oligopeptides that possess an antigenic determinant, or property that interferes, or competes, with the function of a larger polypeptide, and those that affect interactions between IBF and/or IC, other nucleic acid regions, and other proteins. Such sequences may be created by the application of the nucleotides or amino acids being exchanged, deleted, inserted, fused, or otherwise modified. Likewise, the current disclosure within, the sequences that may be naturally present as extensions of, or insertions within, the sequences disclosed herein, including alternative or longer N- and C-terminal sequences, or alternatively spliced protein isoforms.

Production and purification of polypeptides may be achieved in any of a variety of expression systems known to those skilled in the art, including recombinant DNA techniques, genetic recombination, and chemical synthesis. For instance, expression in prokaryotic cells may be achieved by placing protein coding nucleic sequences downstream of a promoter, such as T7, T3, lacI, lacZ, trp, or other cellular, viral, or artificially modified promoters including those that may be inducible by IPTG, tetracycline, maltose, and so forth. Such promoters are often provided for in commercially available recombinant DNA vectors such as pRSET ABC, pBluescript, pKK223-3, and others, or are easily constructed to achieve such a purpose, and often include the presence of multiple cloning sites (MCS) to facilitate typically contain efficient ribosome binding sites, and in some cases transcription termination signals.

Cells for the expression of such proteins are normally E. coli, but could include B. subtilus, B. thuringiensis, B. anthracis, Streptomyces or others prokaryotes. The incorporation of such recombinant DNA can be efficiently achieved by calcium chloride transformation, electroporation, and so forth. In the case of E. coli, cells typically grow in LB media with an appropriate antibiotic selection, for instance ampicillin, chloramphenicol, tetracycline and so forth in order to retain the recombinant vector, although vectors which integrate into the cellular chromosome are also possible. The promoter of many recombinant expression vectors require induction by an inducer compound, for instance IPTG, to facilitate high levels of transcription initiation and subsequent protein production. In some instances, nucleic acid sequences within the coding region may be altered to suit the codon usage patterns of a gives model expression system or organism.

Peptides, oligopeptides and polypeptides may also be produced by chemical synthesis, for instance solid phase techniques, either manually or under automated control such as Applied Biosystems 431 peptide synthesizer (Perkin Elmer). After synthesis, such molecules are often further purified by preparative high performance liquid chromatography. Thus, The disclosure provides methods for the production of epitopes for antibody production, or the production of small molecules that enhance or interfere with a specific function or interaction of the IBF and/or IC polypeptides.

Methods to produce and purify said polypeptides in eukaryotic systems are widely available and understood by those proficient in the art. Cells for such production are known to include yeast and other fungi, Drosophila and Sf9 cells, cells of other higher eukaryotic organisms such as HeLa, COS, CHO and others, as well as plant cells. Similarly, expression could be achieved in prokaryotic or eukaryotic extracts that are able to translate RNAs into proteins, such as rabbit reticulocyte lysates.

### Vectors

Vectors for expression in such systems are widely available both commercially or can be prepared. Such vectors typically are driven by promoters derived from cellular or viral genes, such as CMV, HSV, EBV, HSV, SV40, Adenovirus, LTRs, vaccinia, baculovirus polyhedrin promoter, CaMV, TMV, Rubisco, and so forth, and could obviously include the promoters for the IBF and/or IC genes themselves. Such vectors are often designed be regulated by the presence of enhancer or other regulatory element sequences. Introduction of such vectors into cells is often achieved by calcium phosphate or DEAE dextran technologies, liposome mediated techniques, electroporation, or viral mediated infection. Maintenance of such vectors may be achieved by selectable marker such as that conferred by HSV thymidine kinase, HGPRTase, herbicide resistance, visible markers, and so forth.

Selection of an appropriate methodology would be within the scope of those skilled in such methodologies, using the current disclosure, and would include any combination of host cell and vector which can achieve desired production goals. For instance, the ability of a host cell to drive efficient full-length polypeptide production, glycosylation, membrane anchoring, secretion, absence of contaminating mammalian proteins or infectious agents, proteolytic processing, lipid modification, phosphorylation and so forth may dictate the use of baculovirus/insect cell systems, mammalian cells systems, plant cell systems and so on. In the case of in vitro translation extracts, one embodiment is the coupled transcription and translation of a nonreplicable recombinant vector, where translation is often visualized by the incorporation of a radiolabeled amino acid. The system selected may further depend on the desirability of obtaining purified polypeptides for further characterization, on whether the intent is to evaluate the effect of the overexpressed proteins on cellular gene expression, in vivo or in vitro, to identify compounds that enhance or interfere with the function of the overexpressed polypeptides, or other purposes.

The disclosure also relates to cells which contain such recombinant constructs, where the host cell refers to mammalian, plant, yeast, insect, or other eukaryotic cells, or to prokaryotic, or archae, and vectors that are designed for a given host. Promoter-vector combinations could be chosen by a person skilled in these arts. In some cases, the desired outcome may not be protein, but RNA, and recombinant vectors would include those with inserts present in either forward or reverse orientations.

Many of the vectors and hosts have specific features that facilitate expression or subsequent purification. For instance DNA sequences to be expressed as proteins often appear as fusion with unrelated sequences that encode polyhistidine tags, or HA, FLAG, myc and other epitope tags for immunochemical purification and detection, or phosphorylation sites, or protease recognition sites, or additional protein domains such as glutathione S-transferase (GST), maltose binding protein (MBP), and so forth which facilitate purification. Vectors may also be designed which contain elements for polyadenylation, splicing and termination, such that incorporation of naturally occurring genomic DNA sequences that contain introns and exons can be produced and processed, or such that unrelated introns and other regulatory signals require RNA processing prior to production of mature, translatable RNAs. Proteins produced in the systems described above could be subject to a variety of post-translational modifications, such as glycosylation, phosphorylation, nonspecific or specific proteolysis or processing.

Purification of IBF and/or IC, or variants produced as described above can be achieved by any of several widely available methods. Cells may be subject to freeze-thaw cycles or sonication to achieve disruption, or may be fractionated into subcellular components prior to further purification. Purification may be achieved by one or more techniques such as precipitation with salts or organic solvents, ion exchange, hydrophobic interaction, HPLC and FPLC chromatograpic techniques. Affinity chromatographic techniques could include the use of polyclonal or monoclonal antibodies raised against the expressed polypeptide, or antibodies raised against or available for an epitopic tag such as HA or FLAG. Similarly, purification can be aided by affinity chromatography using fusions to the desired proteins such as GSH-affinity resin, maltose affinity resin, carbohydrate (lectin) affinity resin or, in a one embodiment, Ni-affinity resin, and so forth. In some instances purification is achieved in the presence of denaturing agents such as urea or guanidine, and subsequent dialysis techniques may be required to restore functionality, if desired.

### Antibodies and Antibody Compositions

Antibodies refer to single chain, two-chain, and multi-chain proteins and glycoproteins belonging to the classes of polyclonal, monoclonal, chimeric, and hetero immunoglobulins; it also includes synthetic and genetically engineered variants of these immunoglobulins. “Antibody fragment” includes Fab, Fab′, F(ab′)2, and Fv fragments, as well as any portion of an antibody having specificity toward a desired target epitope or epitopes. A humanized antibody is an antibody derived from a non-human antibody, typically murine, that retains or substantially retains the antigen-binding properties of the parent antibody but which is less immunogenic in humans, U.S. Pat. No. 5,530,101, incorporated herein by reference in its entirety.

An antibody composition of the present disclosure is characterized as containing antibody molecules that immunoreact with IBF and/or IC, or a related polypeptide of this disclosure, but is substantially free of antibodies that immunoreact with any other related protein.

In accordance with the present disclosure, immunoglobulins specifically reactive with IBF and/or IC related epitopes are provided. In accordance with the present disclosure, immunoglobulins specifically reactive with IBF and/or IC related epitopes are provided.

In accordance with the present disclosure, humanized immunoglobulins specifically reactive with IBF and/or IC related epitopes are provided. In accordance with the present disclosure, humanized immunoglobulins specifically reactive with IBF and/or IC related epitopes are provided.

An antibody composition of the present disclosure is typically produced by immunizing a laboratory mammal with an inoculum of the present disclosure and to thereby induce in the mammal antibody molecules having the appropriate polypeptide immunospecificity. The antibody molecules are then collected from the mammal and isolated to the extent desired by well known techniques such as, for example, by immunoaffinity chromatography. The antibody composition so produced can be used in, inter alia, the diagnostic methods and systems of the present disclosure to detect IBF and/or IC in a body sample.

The antibody compositions of this disclosure induced by a polypeptide of this disclosure, including an oligomeric polypeptide and a polypeptide polymer, can be described as being oligoclonal as compared to naturally occurring polyclonal antibodies since they are raised to an immunogen (the relatively small polypeptide) having relatively few epitopes as compared to the epitopes mimicked by an intact IBF and/or IC molecule. Consequently, receptors of this disclosure bind to epitopes of the polypeptide, whereas naturally occurring antibodies raised to the whole IBF and/or IC molecule bind to epitopes throughout the IBF and/or IC molecule and are referred to as being polyclonal.

Monoclonal antibody compositions are also contemplated by the present disclosure. A monoclonal antibody composition contains, within detectable limits, only one species of antibody combining site capable of effectively binding IBF and/or IC. Thus, a monoclonal antibody composition of the present disclosure typically displays a single binding affinity for IBF and/or ICeven though it may contain antibodies capable of binding proteins other than IBF and/or IC.

Suitable antibodies in monoclonal form, typically whole antibodies, can also be prepared using hybridoma technology described by Niman et al., Proc. Natl. Sci., U.S.A., 80:4949-4953 (1983), which description is incorporated herein by reference. Briefly, to form the hybridoma from which the monoclonal antibody composition is produced, a myeloma or other self-perpetuating cell line is fused with lymphocytes obtained from the spleen of a mammal hyperimmunized with a polypeptide of this disclosure.

Splenocytes are typically fused with myeloma cells using polyethylene glycol (PEG) 1500. Fused hybrids are selected by their sensitivity to HAT. Hybridomas secreting the receptor molecules of this disclosure are identified using the enzyme linked immunosorbent assay (ELISA).

A monoclonal antibody composition of the present disclosure can be produced by initiating a monoclonal hybridoma culture comprising a nutrient medium containing a hybridoma that secretes antibody molecules of the appropriate polypeptide specificity. The culture is maintained under conditions and for a time period sufficient for the hybridoma to secrete the antibody molecules into the medium. The antibody-containing medium is then collected. The antibody molecules can then be further isolated by well known techniques.

Media useful for the preparation of these compositions are both well known in the art and commercially available and include synthetic culture media, inbred mice and the like. An exemplary synthetic medium is Dulbecco's minimal essential medium (DMEM; Dulbecco et al., Virol. 8:396 (1959)) supplemented with 4.5 gm/1 glucose, 20 mm glutamine, and 20% fetal calf serum. An exemplary inbred mouse strain is the Balb/c.

The monoclonal antibody compositions produced by the above method can be used, for example, in diagnostic and therapeutic modalities wherein formation of a IBF and/or IC-containing immunoreaction product is desired.

### Diagnostic Systems and Kits

A diagnostic system in kit form of the present disclosure includes, in an amount sufficient for at least one assay, an IBF-IC composition, a polypeptide, antibody composition or monoclonal antibody composition of the present disclosure, as a packaged reagent. Instructions for use of the packaged reagent are also typically included.

As used herein, the term “package” refers to a solid matrix or material such as glass, plastic, paper, foil and the like capable of holding within fixed limits a polypeptide, antibody composition or monoclonal antibody composition of the present disclosure. Thus, for example, a package can be a glass vial used to contain milligram quantities of a contemplated polypeptide or it can be a microtiter plate well to which microgram quantities of a contemplated polypeptide have been operatively affixed, i.e., linked so as to be capable of being immunologically bound by an antibody.

“Instructions for use” typically include a tangible expression describing the reagent concentration or at least one assay method parameter such as the relative amounts of reagent and sample to be admixed, maintenance time periods for reagent/sample admixtures, temperature, buffer conditions and the like.

In preferred embodiments, a diagnostic system of the present disclosure further includes a label or indicating means capable of signaling the formation of a complex containing a polypeptide or antibody molecule of the present disclosure.

The word “complex” as used herein refers to the product of a specific binding reaction such as an antibody-antigen or receptor-ligand reaction. Exemplary complexes are immunoreaction products.

As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an expressed protein, polypeptide, or antibody molecule that is part of an antibody or monoclonal antibody composition of the present disclosure, or used separately, and those atoms or molecules can be used alone or in conjunction with additional reagents such labels are themselves well-known in clinical diagnostic chemistry and constitute a part of this disclosure only insofar as they are utilized with otherwise novel proteins methods and/or systems.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like. A description of immunofluorescence analysis techniques is found in DeLuca, “Immunofluorescence Analysis”, in Antibody As a Tool, Marchalonis, et al., eds., John Wiley & Sons, Ltd., pp. 189-231 (1982), which is incorporated herein by reference.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-azino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. The positrons so emitted produce gamma rays upon encounters with electrons present in the animal's body. Also useful is a beta emitter, such ¹¹¹indium or ³H.

The linking of labels, i.e., labeling of, polypeptides and proteins is well known in the art. For instance, antibody molecules produced by a hybridoma can be labeled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Aurameas, et al., Scand. J. Immunol., Vol. 8 Suppl. 7:7-23 (1978), Rodwell et al., Biotech., 3:889-894 (1984), and U.S. Pat. No. 4,493,795, which are all incorporated herein by reference.

The diagnostic systems can also include, preferably as a separate package, a specific binding agent. A “specific binding agent” is a molecular entity capable of selectively binding a reagent species of the present disclosure or a complex containing such a species, but is not itself a polypeptide or antibody molecule composition of the present disclosure. Exemplary specific binding agents are second antibody molecules, complement proteins or fragments thereof, S. aureus protein A, and the like. Preferably the specific binding agent binds the reagent species when that species is present as part of a complex.

In preferred embodiments, the specific binding agent is labeled. However, when the diagnostic system includes a specific binding agent that is not labeled, the agent is typically used as an amplifying means or reagent. In these embodiments, the labeled specific binding agent is capable of specifically binding the amplifying means when the amplifying means is bound to a reagent species-containing complex.

The diagnostic kits of the present disclosure can be used in an “ELISA” format to detect, for example, the presence or quantity of IBF and/or IC in a body fluid sample such as serum, plasma, or urine, etc. “ELISA” refers to an enzyme-linked immunosorbent assay that employs an antibody or antigen bound to a solid phase and an enzyme-antigen or enzyme-antibody conjugate to detect and quantify the amount of an antigen or antibody present in a sample. A description of the ELISA technique is found in Chapter 22 of the 4th Edition of Basic and Clinical Immunology by D. P. Sites et al., published by Lange Medical Publications of Los Altos, Calif. in 1982 and in U.S. Pat. Nos. 3,654,090; 3,850,752; and 4,016,043, which are all incorporated herein by reference.

Thus, in preferred embodiments, a polypeptide, antibody molecule composition or monoclonal antibody molecule composition of the present disclosure can be affixed to a solid matrix to form a solid support that comprises a package in the subject diagnostic systems.

The reagent is typically affixed to the solid matrix by adsorption from an aqueous medium although other modes of affixation, well known to those skilled in the art, can be used.

Useful solid matrices are also well known in the art. Such materials are water insoluble and include cross-linked dextran; agarose; beads of polystyrene beads about 1 micron to about 5 millimeters in diameter; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

The reagent species, labeled specific binding agent or amplifying reagent of any diagnostic system described herein can be provided in solution, as a liquid dispersion or as a substantially dry power, e.g., in lyophilized form. Where the indicating means is an enzyme, the enzyme's substrate can also be provided in a separate package of a system. A solid support such as the before-described microtiter plate and one or more buffers can also be included as separately packaged elements in this diagnostic assay system.

The packaging materials discussed herein in relation to diagnostic systems are those customarily utilized in diagnostic systems. Such materials include glass and plastic (e.g., polyethylene, polypropylene and polycarbonate) bottles, vials, plastic and plastic-foil laminated envelopes and the like. In one embodiment a diagnostic system of the present disclosure is useful for assaying for the presence of IBF and/or IC. Such a system comprises, in kit form, a package containing an antibody to IBF and/or IC.

### Host Cells

Host cells of the disclosure include, but are not limited to, bacterial cells, such as any Gram-positive, such as Bacillus subtilis, or Gram-negative bacterium, such as Escherichia coli, or any other suitable bacterial strain, fungal cells, such as the yeast Saccharomyces cerevisiae, animal cells, such as hamster, human, or monkey, plant cells, such as Arabidopsis thaliana, or insect cells, such as mosquito, or any other suitable cell. Host cells can be unicellular, or can be grown in tissue culture as liquid cultures, monolayers or the like. Host cells may also be derived directly or indirectly from tissues, such as liver, blood, or skin cells. Vectors can include plasmids, such as pBluescript SK, pBR322, and pACYC 184, cosmids, or virus/bacteriophage, such as pox virus vectors, baculovirus vectors, adenovirus vectors, and lambda, and artificial chromosomes, such as yeast artificial chromosomes (YAC), P1-derived artificial chromosomes (PACs) and bacterial artificial chromosomes (BACs), so long as they are compatible with the host cell, i.e., are stably maintained and replicated. Further, preferred prokaryotic vectors include plasmids such as those capable of replication in E. coli, for example, pBR322, ColE1, pSC101, pACYC 184, etc. (see Maniatis et al., Molecular Cloning: A Laboratory Manuel), Bacillus plasmids such as pC194, pC221, pT127, etc. (Gryczan, T., The Molecular Biology of the Bacilli, Academic Press, NY (1982), pp. 307-329); Streptomyces plasmids including pIJ101 (Kendall, K. J. et al., (1987) J. Bacteriol. 169:4177-4183); Streptomyces bacteriophages such as phiC31 (Chater, K. F. et al., in: Sixth International Symposium on Actinomycetal es Biology, Akademiai Kaido, Budapest, Hungary (1986), pp. 45-54), and Pseudomonas plasmids (John, J. F., et al. (1986) Rev. Infect. Dis. 8:693-704), and Izaki, K. (1978) Jpn. J. Bacteriol. 33:729-742). Preferred eukaryotic plasmids include BPV, vaccinia, SV40, 2-micron circle, etc., or their derivatives. Such plasmids are well known in the art (Botstein, D., et al. (1982) Miami Wint. Symp. 19:265-274; Broach, J. R., in: The Molecular Biology of the Yeast Saccharomyces: Life Cycle and Inheritance, Cold Spring Harbor Laboratory, Cold Spring Harbor, N.Y., pp. 445-470 (1981); Broach, J. R., (1982) Cell 28:203-204; Bollon, D. P., et al. (1980) J. Clin. Hematol. Oncol. 10:39-48; Maniatis, T., in: Cell Biology: A Comprehensive Treatise, Vol. 3: Gene Expression, Academic Press, N.Y., pp. 563-608 (1980)).

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5alpha, JM109, and KC8, as well as a number of commercially available bacterial hosts such as SURE®. Competent Cells and SOLOPACK® Gold Cells (STRATAGENE, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12, etc. Many host cells from various cell types and organisms are available and would be known to one of skill in the art. Similarly, a viral vector may be used in conjunction with either a eukaryotic or prokaryotic host cell, particularly one that is permissive for replication or expression of the vector.

While the disclosure has been described with reference to certain IBF and/or IC proteins, it is to be understood that this covers fragments of the naturally occurring protein and similar proteins with additions, deletions or substitutions which do not substantially affect the immunogenic properties of the recombinant protein.

### Therapeutic Agents

The disclosure provides for the administration of the IBF-IC vaccine prior to, concurrently with, subsequent to, or in combination with, for example, one or more therapeutic agent. The term “therapeutic agent” includes agents that are useful for the treatment of a disease or a physiological condition in an animal (e.g., a mammal such as a human) and thus includes known drugs. Thus, the term “therapeutic agent” includes but is not limited to known drugs and/or drugs that have been approved for sale in the United States. For example, therapeutic agents include but are not limited to chemotherapeutic agents, antibiotic agents, antifungal agents, antiparasitic agents and antiviral agents. The term “therapeutic agent” agent also includes “prodrugs” of such therapeutic agents or drugs. The term “therapeutic agent” agent also includes functional group derivatives of such therapeutic agents or drugs. Accordingly, the term “therapeutic agent” includes a therapeutic agent, a prodrug of a therapeutic agent and a functional group derivatives of therapeutic agent.

Examples of antifungal agents within the scope of the present disclosure include for example polyene, azole, allylamine, morpholine, and antimetabolite antifungal agents, e.g., amphotericin B, candicin, filipin, hamycin, natamycin, nystatin rimocidin, bifonazole, butoconazole, clotrimazole, econozole, fenticonazole, isoconazole, ketoconazole, luiconazole, miconazole, omoconazole, oxiconazole, sertaconazole, sulconazole, tioconazole, albaconazole, fluconazole, isavuconazole, traconazole, posaconazole, ravuconazole, terconazole, voriconazole, abafungin, amorolfin, butenafine, naftifine, terbinafine, anidulafungin, caspofungin, micafungin, benzoic acid, ciclopirox, griseofulvin, tolnaftate, and undecylenic acid.

Examples of antibiotic agent within the scope of the present disclosure include for example aminoglycosides (e.g., amikacin, gentamicin, kanamycine, neomycine, metilmicin, tobramycin, paromomycin, streptomycin, spectinomycin), anasamycins (e.g., geldanamycin, herbimycin, riflaximin), loracerbef, carbapenems (e.g., ertapenem, doripenem, cilastatin, meropenem), cephalosporin (e.g. cefadroxil, cefazolin, cephalexin, cefaclor, cefamandole, cefoxitin, cefprozil, cefuroxime, cefixime, cefdinir, cefdotoren, cefotaxime, ceftibuten, ceftizoxime, cefepime, ceftaroline, ceftobioprole, teichoplanin, vancomycin, telavancin, clindamycin, lincomycin, daptomycin, azithromycin, clarithromycin, dirithromycin, erythromycin, roxithromycin, troleandomycin, telithromycin, spiramycin, azetreonam, flurazolidone, linezolid, posizolid, radezolid, torezolid, ampicillin, azolocillin, carbenicillin, cloxacillin, dicloxaxillin, pencillin), polypeptides (e.g. bacitracin, colistin, polymyxin B), Quinolones (e.g., ciproflaxin, enoxacin, gemifloxacin, norfloxacin), sulfonamides (e.g., malfenide, sulfamethizole, sulfasalazine, sulfadiazine), tetracyclines (e.g., demeclocycline, minocycline, doxycycline, tetracycline), clofazimine, dapsone, capreomycin, cycloserine, ethambutol, ethionamide, isoniazid, pyrazinamide, riflampicin, rifabutin, rifapentine, streptomycin, arsphenamine, chloramthenicol, foffmycin, fusidic acid, metronidazole, mupirocin, platensimycin, thiamphenicol, tigecycline, tinidazole, and trimethoprim.

Examples of antiviral drugs within the scope of the present disclosure include for example Abacavir, Aciclovir, Acyclovir, Adefovir, Amantadine, Amprenavir, Ampligen, Arbidol, Atazanavir, Atripla (fixed dose drug), Boceprevir, Cidofovir, Combivir (fixed dose drug), Darunavir, Delavirdine, Didanosine, Docosanol, Edoxudine, Efavirenz, Emtricitabine, Enfuvirtide, Entecavir, Entry inhibitors, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Stavudine, Synergistic enhancer (antiretroviral), Tea tree oil, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine and the like.

Examples of non-opioid and anti-inflammatory agents within the scope of the present disclosure include for example acetaminophen, aspirin, diflunisal, choline magnesium trisalicylate, salsalate, ibuprofen, naproxen, ketoprofen, fluriprofen, oxaprozin, indomethacin, sulindac, nabumetone, diclofenac, ketorolac, tolectin, piroxicam, meloxicam, mefenamic acid, meclofenamate, celecoxib, allopurinol, dextromethorphan, pegloticase, dexibuprofen, etodolac, fenoprofen, flufenamic acid, flupbiprofen, lornoxicam, loxoprofen, meclofenamic acid, piroxicam, tenoxicam, tolmetin, and tolfenamic acid.

Examples of immunosuppresive agents within the scope of the present disclosure include for example alkylating agents, antimetabolites, high dose corticosteroids, azathioprine, mycophenolate mofetil, cyclosporine, methotrexate, leflunomide, cyclophosphamide, chlorambucil, nitrogen mustard, abacavir, abciximab, adalimumab, aldesleukin, altretamine, aminoglutethimide, amprevenir, anakinra, anastrozole, aspariginase, azathioprine, basiliximab, betamethasone, bleomycin, busulfan, capecitabine, carboplatin, carmustine, chlorambucil, cidofovir, cisplatin, cladribine, cortisone, cyclosporine, cytarabine, decarbazine, dacuzumab, dactinomycin, daunorubicin, delaviridine, dexamethasone, didanosine, doxorubicin, efavirenz, epirubicin, estramustine, etanercept, etoposide, exemestane, foxuridine, fludarabine, fluorouracil, flutamide, gemcitabine, gemtuzumab ozogamicin, hydrocortisone, hydroxychloroquine, hydroxyurea, idaubicin, ifosphamide, indinavir, infliximab, interferon alpha-2a, interferon alpha-2b, interferon beta-2b, interferon beta-2a, interferon gamma-1b, interleukin-2, irinotecan, isotretinoin, lamivudine, leflunomide, letrozole, leuprolide, mechloethamine, megestrol, melphalan, mercaptopurine, methotrexate, methylpregnisolone, mitomycin, mitotane, mitoxantrone, mycophenolate, nelfinavir, nevirapine, paclitaxel, pegaspargase, penicillamine, pentostatin, pimecroslimus, pipobroman, plicamycin, prednisolone, predisone, priliximab, procarbazine, ritonavir, rituximab, saquinavir, sargamomstim, stavudine, strepozocin, tacrolismus, temozolomide, teniposide, testolactone, thioguanine, thiotepa, trastuzumab, tretinoin, triamcinolone, uracil mustard, valrubucin, vinblastine, vincristine, vinorelbine, zalcitabine, zidovudine, metronidazole, mupirocin, platensimycin, thiamphenicol, tigecycline, tinidazole, and trimethoprim.

Examples of anti-cancer drugs within the scope of the present disclosure include for example bortezomib ([(1R)-3-methyl-1-[[(2S)-1-oxo-3-phenyl-2-[(pyrazinylcarbonyl) amino]propyl]amino]butyl] boronic acid; MG-341; VELCADE), MG-132 (N-[(phenylmethoxy)carbonyl]-L-leucyl N-[(1S)-1-formyl-3-methylbutyl]-L-leucinamide); pyrimidine analogs (e.g., 5-fluorouracil, floxuridine, capecitabine, gemcitabine and cytarabine); purine analogs; folate antagonists and related inhibitors (e.g., mercaptopurine, thioguanine, pentostatin and 2-chlorodeoxyadenosine [cladribine]); folic acid analogs (e.g., methotrexate); antimitotic agents, including vinca alkaloids (e.g., vinblastine, vincristine, and vinorelbine) and alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC); microtubule disruptors (e.g., paclitaxel, docetaxel, vincristin, vinblastin, nocodazole, epothilones and navelbine, and teniposide); actinomycin, amsacrine, anthracyclines, bleomycin, busulfan, camptothecin, carboplatin, chlorambucil, cisplatin, cyclophosphamide, Cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP 16); dactinomycin (actinomycin D), daunorubicin, doxorubicin (adriamycin), idarubicin, anthracyclines, mitoxantrone, bleomycins, plicamycin (mithramycin) and mitomycin; L-asparaginase; antiplatelet agents; platinum coordination complexes (e.g., cisplatin, carboplatin), procarbazine, hydroxyurea, mitotane, aminoglutethimide; hormones and hormone analogs (e.g., estrogen, tamoxifen, goserelin, bicalutamide, nilutamide); aromatase inhibitors (e.g., letrozole, anastrozole); anticoagulants (e.g., heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (e.g., breveldin); immunosuppressives (e.g., cyclosporine, tacrolimus (FK-506), sirolimus (rapamycin), azathioprine, mycophenolate mofetil); anti-angiogenic compounds (e.g., TNP-470, genistein) and growth factor inhibitors (e.g., vascular endothelial growth factor (VEGF) inhibitors, fibroblast growth factor (FGF) inhibitors, epidermal growth factor (EGF) inhibitors); angiotensin receptor blockers; nitric oxide donors; anti-sense oligonucleotides; antibodies (e.g., trastuzumab (HERCEPTIN), AVASTIN, ERBITUX); cell cycle inhibitors and differentiation inducers (e.g., tretinoin); mTOR (mammalian target of rapamycin) inhibitors (e.g., everolimus, sirolimus); topoisomerase inhibitors e.g., doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan); corticosteroids (e.g., cortisone, dexamethasone, hydrocortisone, methylpednisolone, prednisone, and prenisolone); growth factor signal transduction kinase inhibitors; mitochondrial dysfunction inducers; and caspase activators and the like.

Examples of anti-cancer drugs within the scope of the present disclosure include for example alemtuzumab; aminoglutethimide; amsacrine; anastrozole; asparaginase; bevacizumab; bicalutamide; bleomycin; bortezomib; buserelin; busulfan; campothecin; capecitabine; carboplatin; carmustine; CeaVac; cetuximab; chlorambucil; cisplatin; cladribine; clodronate; colchicine; cyclophosphamide; cyproterone; cytarabine; dacarbazine; daclizumab; dactinomycin; daunorubicin; dienestrol; diethylstilbestrol; docetaxel; doxorubicin; edrecolomab; epirubicin; epratuzumab; erlotinib; estradiol; estramustine; etoposide; exemestane; filgrastim; fludarabine; fludrocortisone; fluorouracil; fluoxymesterone; flutamide; gemcitabine; gemtuzumab; genistein; goserelin; huJ591; hydroxyurea; ibritumomab; idarubicin; ifosfamide; IGN-101; imatinib; interferon; irinotecan; ironotecan; letrozole; leucovorin; leuprolide; levamisole; lintuzumab; lomustine; MDX-210; mechlorethamine; medroxyprogesterone; megestrol; melphalan; mercaptopurine; mesna; methotrexate; mitomycin; mitotane; mitoxantrone; mitumomab; nilutamide; nocodazole; octreotide; oxaliplatin; paclitaxel; pamidronate; pentostatin; pertuzumab; plicamycin; porfimer; procarbazine; raltitrexed; rituximab; streptozocin; sunitinib; suramin; tamoxifen; temozolomide; teniposide; testosterone; thalidomide; thioguanine; thiotepa; titanocene dichloride; topotecan; tositumomab; trastuzumab; tretinoin; vatalanib; vinblastine; vincristine; vindesine; and vinorelbine and the like.

### Pharmaceutical Compositions and Administration

Administration of therapeutically effective amounts of an IBF and/or IC vaccine and/or additional therapeutic agent/s, is by any of the routes normally used for introducing protein or encoding nucleic acids into ultimate contact with the tissue to be treated. The protein or encoding nucleic acids are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington's Pharmaceutical Sciences, 17th ed. 1985)).

The protein or encoding nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064. Treated stem cells can be returned to a patient fortreatment of various diseases including, but not limited to, SCID and sickle-cell anemia.

### Dosages

The amount or dose of IBF-IC can be adjusted to desired clinical outcomes. “(The Merck Manual of Diagnosis and Therapy, thirteenth edition, 1977). Principles for the use of drugs affecting the immune response are described; e.g. as to corticosteroids Merck Manual, 1977, states at page 1903:” 8. All dosages should be individualized. The effective dose varies with different diseases, with different phases of the same disease, and from patient to patient. 9. The dosage should be kept flexible, being raised or lowered according to alterations in the course of the disease or the development of undesirable effects.” The type of IC amount and antigen in a given patient can vary considerably. It is a particular advantage of the disclosure that the therapy can be specifically designed for the conditions and needs of an individual patient.

For therapeutic applications, the dose administered to a patient, or to a cell which will be introduced into a patient, in the context of the present disclosure, should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy and Kd of the particular compound employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or vector in a particular patient.

In determining the effective amount of the compound which modulates, for example, IBF and/or IC expression or activity to be administered in the treatment or prophylaxis of disease, the physician evaluates circulating plasma levels of the compound which modulates, for example, IBF and/or IC expression or activity or nucleic acid encoding the compound which modulates expression, potential compounds which modulates, for example, IBF and/or IC toxicities, progression of the disease, and the production of antibodies. Administration can be accomplished via single or divided doses.

### ADDITIONAL REFERENCES

ABCAM. Using IgM antibodies for IP. www.abcam.com/protocols/immunoprecipitation-protocol-1. Downloaded Mar. 22, 2018.
Acchione M. et al. Impact of linker and conjugation chemistry on antigen binding, Fc receptor binding and thermal stability of model antibody-drug conjugates. mAbs, 4:3, 362-372, DOI: 10.4161/mabs.19449. 2012.
Karen S. Anderson, Joshua LaBaer. The sentinel within: exploiting the immune system for cancer biomarkers. Proteome Res. 2005; 4(4): 1123-1133.
Bartkowiak B. et al. Crosslinking Primary Antibody to Protein A/G resin using dimethylpimelimidate (DMP), for MS-Compatible Immunoprecipitation. Duke Proteomics Core. May 2012.
Anne Baerenwaldta, Anja Luxa, Heike Danzera, Bernd M. Spriewaldb, Evelyn Ullrichb, Gordon Heidkampc, Diana Dudziakc, Falk Nimmerjahna. Fcγ receptor IIB (FcγRIIB) maintains humoral tolerance in the human immune system in vivo. PNAS, 2011, 108, 46, 18772-18777.
Edward Bernton, David Haughey. Studies of the Safety, Pharmacokinetics and Immunogenicity of Repeated Doses of Intravenous Staphylococcal Protein A in Cynomolgus Monkeys. Basic & Clinical Pharmacology & Toxicology, 2014, 115, 448-455.
Edward Bernton, William Gannon, William Kramer, and Eduard Kranz. PRTX-100 and Methotrexate in Patients With Active Rheumatoid Arthritis: A Phase 1b Randomized, Double-Blind, Placebo-Controlled, Dose-Escalation Study. Clinical Pharmacology in Drug Development.
L Bracci, G Schiavoni, A Sistigu and F Belardelli. Immune-based mechanisms of cytotoxic chemotherapy: implications for the design of novel and rationale-based combined treatments against cancer. Cell Death and Differentiation (2014) 21, 15-25.
Barkas T. A simple, rapid and sensitive assay for immune complexes using a Staphylococcus aureus immunoadsorbent. J Clin Lab Immunol. 1981 January; 5(1):59-65.
Darren R. Brenner, Dominique Scherer, Kenneth Muir, Joellen Schildkraut, Paolo Boffetta, Margaret R. Spitz, Loic Le Marchand, Andrew T. Chan, Ellen L. Goode, Cornelia M.
Ulrich, Rayjean J. Hung. A Review of the Application of Inflammatory Biomarkers in Epidemiologic Cancer Research. Downloaded from cebp.aacrjournals.org on Jun. 18, 2016. © 2014 American Association for Cancer Research.
Bijsterbosch M K. Et al. Crosslinking Of Surface Immunoglobulin And Fc Receptors On B Lymphocytes Inhibits Stimulation Of Inositol Phospholipid Breakdown Via The Antigen Receptors. J. ExP. MED. Volume 162 December 1985. P. 1825-1836.
Blood plasma fractionation en.wikipedia.org/wiki/Blood_plasma_fractionation
Bruhns et al. Specificity and affinity of human Fcgamma receptors and their polymorphic variants for human IgG subclasses. Blood. 2009 Apr. 16; 113(16):3716-25.
Cheung et al. Measurement of circulating immune complexes in human sera by enzyme immunoassay. Aust J Exp Biol Med Sci. 1982 December; 60(6):593-605.
Anissa S. H. Chan, Adria Bykowski Jonas, Xiaohong Qiu, Nadine R. Ottoson, Richard M. Walsh, Keith B Gorden, Ben Harrison, Peter J. Maimonis, Steven M. Leonardo, Kathleen E. Ertelt, Michael E. Danielson, Kyle S. Michel, Mariana Nelson, Jeremy R. Graff, Myra L. Patchen, Nandita Bose* Imprime PGG-Mediated Anti-Cancer Immune Activation Requires Immune Complex Formation. PLOS ONE|DOI:10.1371/journal.pone.0165909 Nov. 3, 2016.
Anil K. Chauhan. Human CD4+ T-Cells: A Role for Low-Affinity Fc Receptors. Frontiers in Immunology|www.frontiersin.org June 2016 |Volume 7 |Article 215.
Campiat S, Dercle L, Ammari S, et al. Hyperprogressive disease (HPD) is a new pattern of progression in cancer patients treated by anti-PD-1/PD-L1. Clin Cancer Res. 2016; 23(8):1920-1928. doi: 10.1158/1078-0432.ccr-16-1741.
Lisa M. Coussens, Zena Werb. Inflammation and cancer. Nature 420, 860-867 2002.
Fred M. Cowan. Editorial Connecting science past and future. Journal of the Chinese Medical Association Volume 80, Issue 1, January 2017, Page 1-2.authors.elsevier.com/sd/article/S1726-4901(16)30184-8
Cowan, F. M. A Method for Treating Cellular Fc Receptor Mediated Immune Disorders. U.S. Pat. No. 5,189,014, Feb. 23, 1993 (original application filled 1979).
Cowan, F. M., Klein, D. L., Armstrong, G. R., Stylos, W. A. and Pearson, J. W. (1980). Fc receptor mediated immune regulation and gene expression. Biomedicine, 32, 108-110.
Cowan, F M, Broomfield, C A, Stojiljkovic, M P, Smith, W J. A Review of Multi-Threat Countermeasures against Chemical Warfare and Terrorism. MILITARY MEDICINE, 169, 11:850, 2004.
Brendon J Coventry, Martin L Ashdown. Complete clinical responses to cancer therapy caused by multiple divergent approaches: a repeating theme lost in translation. Cancer Management and Research 2012:4 137-149.
Rony Dahan, Emanuela Sega, John Engelhardt, Mark Selby, Alan J. Korman, Jeffrey V. Ravetch, FcgRs Modulate the Anti-tumor Activity of Antibodies Targeting the PD-1/PD-L1 Axis. Cancer Cell 28, 285-295, Sep. 14, 2015.
Daniel M. Czajkowsky, Jun Hu, Zhifeng Shaol, Richard J. Pleass. Fc-fusion proteins: new developments and future perspectives. EMBO Mol Med (2012) 4, 1015-1028.
Kavita M. Dhodapkar, Jacob L. Kaufman, Marc Ehlers, Devi K. Banerjee, Ezio Bonvini, Scott Koenig, Ralph M. Steinman, Jeffrey V. Ravetch, Madhav V. Dhodapkar. Selective blockade of inhibitory Fc_receptor enables human dendritic cell maturation with IL-12p70 production and immunity to antibody-coated tumor cells. PNAS, 2005, 102-8, 2910-2915.
Charles A. Dinarello. Anti-inflammatory Agents: Present and Future. Cell 140, 935-950, Mar. 19, 2010.
S. J. Drakeley, P. N. Furness. Heparin inhibits the binding of immune complexes to cultured rat mesangial cells. Nephrol Dial Transplant (1994) 9: 1084-1089.
Dima, Sjoquist et al (1983). Effect of protein A and its fragment B on the catabolic and Fc receptor sites of IgG. European Journal of Immunology, 1983, Volume 13, Issue 8, pages 605-614.
Paula Fernandez-Vizarra, Oscar Lopez-Franco, Ben at Mallavia, Alejandro Higuera-Matas,
Virginia Lopez-Parra, 1 Guadalupe Ortiz-Muñoz, Emilio Ambrosio, Jesus Egido, Osborne F. X. Almeida and Carmen Gomez-Guerrero. Immunoglobulin G Fc receptor deficiency prevents Alzheimer-like pathology and cognitive impairment in mice. Brain 2012: 135; 2826-2837.
Rafal Fudala, Agnieszka Krupa, Michael A. Matthay, Timothy C. Allen, Anna K. Kurdowska. Anti-IL-8 autoantibody:IL-8 immune complexes suppress spontaneous apoptosis of neutrophils. Am J Physiol Lung Cell Mol Physiol 293: L364-L374, 2007.
Fukuda et al. Modified Anti-C3 Immune Complex Assay Which Avoids Interference by Anti-F(ab′)2 Antibodies. The Tohoku Journal of Experimental Medicine. Vol. 146. 1985, p. 337-347.
GE Healthcare Life Sciences. Achieving self-sufficiency. Securing a reliable supply of fractionated blood plasma for your needs. 29-0164-85 AA 09/2012.
Gyka G et al. Crosslinkage of antibodies to staphylococcal protein A matrices. Journal of Immunological Methods, Vol. 57, Issue 1-3, pp. 227-233, Feb. 1, 1983.
Saito Yuki Hamano, Hisashi Arase, Hiromitsu Saisho, Takashi. Vivo Th Cell Responses Augmentation of Antigen Presentation for in Immune Complex and Fc Receptor-Mediated.
J Immunol 2000; 164:6113-6119.
Hellström K E, Hellström I, Snyder H W Jr, Balint J P, Jones F R. Blocking (suppressor) factors, immune complexes, and extracorporeal immunoadsorption in tumor immunity. Contemp Top Immunobiol. 1985; 15:213-38.
Hogarth P M, Pietersz G A. Fc receptor-targeted therapies for the treatment of inflammation, cancer and beyond. Nat Rev Drug Discov. 2012; 11:3Ile31
Immobilization of Antibody on Beads.
www.cd-bioparticles.com/s/Immobilization-of-Antibody-on-Beads_40.html
Ingavle G C. Et al. Affinity binding of antibodies to supermacroporous cryogel adsorbents with Immobilized protein A for removal of anthrax toxin protective antigen. Biomaterials. 50 (2015) 140-153.
Cynthia Jane, A. V. Nerurkar, F. R. Karjodkar. CIRCULATING IMMUNE COMPLEXES (CIC) AS MARKER FOR DISEASE PROGRESS IN ORAL CANCER. Indian Journal of Clinical Biochemistry, 2007/22 (2) 114-117.
Jessy T. Immunity over inability: The spontaneous regression of cancer. J Nat Sc Biol Med 2011; 2:43-9.
Hsin-Hsuan Juo, Edward Chiou, Keith B. Elkon and Christian Lood. Immune Complex-Driven Neutrophil Activation in Systemic Lupus Erythematosus—Novel Biomarkers of Disease Activity and Severity. ABSTRACT NUMBER: 2641 2017 ACR/ARHP Annual Meeting Sep. 18, 2017.
Orsolya Kiraly, Guanyu Gong, Werner Olipitz, Sureshkumar Muthupalani, Bevin P. Engelward. Inflammation-Induced Cell Proliferation Potentiates DNA Damage-Induced Mutations In Vivo. PLOS Genetics|DOI:10.1371/journal.pgen.1004901 Feb. 3, 2015 1/24
Junker F, Krishnarajah S, Qureshi O, Humphreys D, Fallah-Arani F. A simple method for measuring immune complex-mediated, Fc gamma receptor dependent antigen-specific activation of primary human T cells. J Immunol Methods. 2018 March; 454:32-39. doi: 10.1016/j.jim.2017.12.002. Epub 2017 Dec. 16.
Kapoor M. How to cross-link proteins. Mar. 13, 2015. www.researchgate.net/publication/267368798_How_to_cross-link_proteins
Knighton. Separation of Blood Proteins. www.mirinz.org.nz/docs/blood-protein-separation.pdf.
Jennifer Lambour, Mar Naranjo-Gomez, Marc Piechaczyk, Mireia Pelegrin. Converting monoclonal antibody-based immunotherapies from passive to active: bringing immune complexes into play. Emerging Microbes & Infections, 2016.
Clifton Leaf The Truth in Small Doses: Why We're Losing the War on Cancer—and How to Win It.
Léonetti M1, Galon J, Thai R, Sautès-Fridman C, Moine G, Menez A. Presentation of antigen in immune complexes is boosted by soluble bacterial immunoglobulin binding proteins. J Exp Med. 1999 Apr. 19; 189(8):1217-28.
Yun Lin, Li Zhang, Ann X. Cai, Mark Lee, Wandi Zhang, Donna Neuberg, Christine M. Canning, Robert J. Soiffer, Edwin P. Alyea, Jerome Ritz, Nir Hacohen, Terry K. Means, Catherine J. Wu. Effective posttransplant antitumor immunity is associated with TLR-stimulating nucleic acid-immunoglobulin complexes in humans. The Journal of Clinical Investigation 121-4, 1574-84, 2011.
J. D. Loike, D. Y. Shabtai, R. Neuhut, S. Malitzky, E. Lu, J. Husemann, I. J. Goldberg, S. C. Silverstein. Statin Inhibition of Fc Receptor-Mediated Phagocytosis by Macrophages Is Modulated by Cell Activation and Cholesterol. Arterioscler Thromb Vasc Biol. 2004, 2051-56.
Levinson et al. Evaluation of anti-Clq capture assay for detecting circulating immune complexes and comparison with polyethylene glycol-immunoglobulin G, Clq-binding, and Raji cell methods. J Clin Microbiol. 1987. P. 1567-1569.
Lindsay M. MacLellan, Jennifer Montgomery, Fujimi Sugiyama, Susan M. Kitson, KatjaThummler, Gregg J. Silverman, Stephen A. Beers, Robert J. B. Nibbs, lain B. McInnes, and Carl S. Goodyear. Co-Opting Endogenous Immunoglobulin for the Regulation of Inflammation and Osteoclastogenesis in Humans and Mice. Arthritis Rheum. 2011 December; 63(12): 3897-3907.
Ralph Moss. HISTORY OF IMMUNE CHECKPOINT INHIBITION. www.ralphmossblog.com/2017/02/history-of-immune-checkpoint-inhibition.html
Melin et al. Normal serum levels of immune complexes in postpolio patients. Results Immunol. 2014; 4:54-57.
Mellencamp et al. Isolation and characterization of circulating immune complexes from patients with pneumococcal pneumonia. Infect Immun. 1987 August; 55(8):1737-42.
Patel et al. Isolation and dissociation of immune complexes from pleural effusions oflymphoma patients. Cancer Immunology, Immunotherapy. September 1986, Volume 23, Issue 1, pp 51-55
Andres Peisajovich, Lorraine Marnell, Carolyn Mold, Terry W Du Clos. C-reactive Protein at the Interface Between Innate Immunity and Inflammation. Expert Rev Clin Immunol. 2008; 4(3):379-390.
C. Prull, “Part of a Scientific Master Plan” Paul Ehrlich and the Origins of his Receptor Concept,” 2003, Medical History 47: 332-356.
Barbara Platzer, Madeleine Stout, Edda Fiebiger. Antigen cross-presentation of immune complexes. www.frontiersin.org April 2014|Volume 5|Article 140|1
Khadija Rafiq, Amy Bergtold, Raphael Clynes. Immune complex-mediated antigen presentation induces tumor immunity. J. Clin. Invest. 110:71-79 (2002).
Elizabeth R. Rayburn, Scharri J. Ezell, and Ruiwen Zhang. Anti-Inflammatory Agents for Cancer Therapy. Mol Cell Pharmacol. 2009; 1(1): 29-43.
Saverino D, Simone R, Bagnasco M, Pesce G. The soluble CTLA-4 receptor and its role in autoimmune diseases: an update. Auto Immun Highlights. 2010 Nov. 4; 1(2):73-81.
Shabnam Shalapour, Michael Karin. Immunity, inflammation, and cancer: an eternal fight between good and evil. jci.org Volume 125 Number 9 Sep. 2015.
N. R. StC. Sinclair, “Why so many coinhibitory receptors?,” Scandinavian Journal of Immunology, 50(1): 10-3 Jul. 1999.
Sinclair N R StC. Immunoregulation by Antibody and Antigen-Antibody Complexes. Transplant. Proc. 1978, 10, 349-53.
Sinclair N R StC. & Panoskaltsis A. Immunoregulation by Fc signals A mechanism for self-nonself discrimination. Immunol Today. 1987; 8(3):76-9. doi: 10.1016/0167-5699(87)90849-8.
H. O. Sjögren, I. Hellström, S. C. Bansal, and K. E. Hellström. Suggestive Evidence That the “Blocking Antibodies” of Tumor-Bearing Individuals May Be Antigen-Antibody Complexes. Proc. Nat. Acad. Sci. USA Vol. 68, 6, 1372-1375, June 1971.
Mark J Smyth, Shin Foong Ngiow, Michele W L Teng. Using FcR to suppress cancer. Targeting regulatory T cells in tumor immunotherapy. Immunology and Cell Biology (2014) 92, 473-474.
Takemasa Tsuji, Sacha Gnjatic. Split T-cell tolerance as a guide for the development of tumor antigen-specific immunotherapy. Oncolmmunology 1:3, 40 406, 2012.
David S. Terman. PROTEIN A AND STAPHYLOCOCCAL PRODUCTS IN NEOPLASTIC DISEASE. CRC Critical Reviews in Ontology˜Hematology Volume 4, Issue 2 103-124, 1985.
Ingeborg Tinhofer, Volker Budach, Korinna Jöhrens and Ulrich Keilholz. The rationale for including immune checkpoint inhibition into multimodal primary treatment concepts of head and neck cancer. Tinhofer et al. Cancers of the Head & Neck (2016) 1:8.
Suzanne L. Topalian, Charles G. Drake, Drew M. Pardoll. Immune Checkpoint Blockade: A Common Denominator Approach to Cancer Therapy. Cancer Cell 27, Apr. 13, 2015.
U.S. 2008/0096233 (Robotti et al.)
Senbagavalli et al. Immune Complexes Isolated from Patients with Pulmonary Tuberculosis Modulate the Activation and Function of Normal Granulocytes. Clin Vaccine Immunol. 2012 December; 19(12): 1965-1971.
Sisson T H. et al. An improved method for immobilizing IgG antibodies on protein A-agarose. Journal of Immunological Methods, 127 (1990) p. 215-220.
Teshima et al. Circulating immune complexes detected by 125I-Clq deviation test in sera of cancer patients. J Clin Invest. 1977 June; 59(6): 1134-1142.
ThermoFisher Scientific. Avidin-Biotin Interaction.
Theofilopoulos et al. Isolation of circulating immune complexes using Raji cells. Separation of antigens from immune complexes and production of antiserum. J Clin Invest. 1978 June; 61(6): 1570-1581.
Theofilopoulos et al. Immune complexes in human diseases: a review. Am J Pathol. 1980 August; 100(2): 529-594.
Steindl et al. A simple method to quantify staphylococcal protein A in the presence of human or animal IgG in various samples. J Immunol Methods. 2000 Feb. 21; 235(1-2):61-9.
U.S. Pat. No. 7,211,258, “Protein A compositions and methods of use” filed 2002 and issued with method of treatment claims for RA, juvenile RA, and systemic lupus erythematosus (SLE).
Viosin G A. Immunity and Tolerance a Unified Concept. Cellular Immunology 1971, 2, 670-89.
Lisa T. C. Vogelpoel, Dominique L. P. Baeten, Esther C. de Jong, Jeroen den Dunnen. Control of cytokine production by human Fc gamma receptors: implications for pathogen defense and autoimmunity. FrontiersinImmunology. Inflammation February 2015 6, Article 79 2.
Anna Wasiuk, James Testa, Jeff Weidlick, Crystal Sisson, Laura Vitale, Jenifer Widger, Andrea Crocker, Lawrence J. Thomas, Joel Goldstein, Henry C. Marsh, Tibor Keler, Li-Zhen He.
CD27-Mediated Regulatory T Cell Depletion and Effector T Cell Costimulation Both Contribute to Antitumor Efficacy. The Journal of Immunology, 2017, 199: 4110-4123.
David J. J. Waugh, CatherineWilson. The Interleukin-8 Pathway in Cancer. Clin Cancer Res 2008, 6735 14(21) 2008.
Xuexiang Du, Fei Tang, Mingyue Liu, Juanjuan Su, Yan Zhang, Wei Wu, Martin Devenport, Christopher A Lazarski, Peng Zhang, Xu Wang, Peiying Ye, Changyu Wang, Eugene Hwang, Tinghui Zhu, Ting Xu, Pan Zheng, Yang Liu. A reappraisal of CTLA-4 checkpoint blockade in cancer immunotherapy. Cell Research (2018) 0:1-17; doi.org/10.1038/s41422-018-0011-0
Yu-mei Wen, Libing Mu, Yan Shi. Immunoregulatory functions of immune complexes in vaccine and therapy. EMBO Molecular Medicine. Published online: Aug. 29, 2016.
Zengguang Xu, Fengying Wu, Chunhong Wang, Xiyu Liu, Baoli Kang, Shan Shan, Xia Gu, Kailing Wang, Tao Ren. The stimulatory activity of plasma in patients with advanced non-small cell lung cancer requires TLR-stimulating nucleic acid immunoglobulin complexes and discriminates responsiveness to chemotherapy. Xu et al. Cancer Cell International 2014, 14:80 www.cancerci.com/content/14/1/80.
Lab Corp Immune Complex Panel TEST: 054494 commercial test www.labcorp.com/test-menu/29291/immune-complexes-profile#
WhatIsBiotechnology checkpoint historic timeline. www.whatisbiotechnology.org/index.php/science/summary/checkpoint-inhibitor/immune-checkpoint-inhibitors-are-key-cancer-treatment-tool
History of Immune Checkpoint Inhibition. Moss R W. www.mossreports.com/history-of-immune-checkpoint-inhibition/
Genocea Unveils Possible Mechanism for Tumor Resistance to Immunotherapy at Society for Immunotherapy of Cancer's (SITC) 33rd Annual Meeting. Nov. 6, 2018 08:00 ET Source: Genocea Biosciences, Inc. ATLAS™-identified “inhibitory” neoantigens promote tumor growth in mouse model. www.globenewswire.com/news-release/2018/11/06/164594I/O/en/Genocea-Unveils-Possible-Mechanism-for-Tumor-Resistance-to-Immunotherapy-at-Society-for-Immunotherapy-of-Cancer-s-SITC-33rd-Annual-Meeting.html
Nobel Prize press release: www.nobelprize.org/prizes/medicine/2018/press-release/
Batchelor, J R. The Use of Enhancement in Studying Tumor Antigens. Cancer Res 1968; 28:1410-1414.
Biguzzi S. Interaction of anti-staphylococcal protein A antisera with Fc receptor-bearing human normal lymphocytes. Eur J Immunol. 1979 January; 9(1):52-60.
Cabel L, Proudhon C, Romano E, Girard N, Lantz O, Stern M-H, Pierga J-Y, Bidard F C. Clinical potential of circulating tumour DNA in patients receiving anticancer immunotherapy Nature Reviews Clinical Oncologyvolume 15, pages 639-650 (2018).
Choe W, Durgannavar T A, Chung S J. Fc-Binding Ligands of Immunoglobulin G. An Overview of High Affinity Proteins and Peptides. Materials 2016, 9, 994; doi:10.3390/ma9120994.
Connelly S. Progression Issues Surface With NSCLC Immunotherapy. OncologyLive—Vol. 18/No. 20. Published: Friday, Oct. 27, 2017. (ct)DNA hyperprogression and efficacy. www.onclive.com/publications/oncology-live/2017/vol-18-no-20/progression-issues-surface-with-nsclc-immunotherapy?p=2
Cowan F M, Klein D L, Armstrong G R, Ablashi D V, Pearson J W, Bensinger W I. Inhibition of rat adenocarcinoma metastases by “Staphylococcus aureus” protein A. Biomed Pharmacother. 1982 January; 36(1):29-31.
Eftimiadi G, Vinai P, Eftimiadi C. Staphylococcal Protein A as a Pharmacological Treatment for Autoimmune Disorders. 2017 J Autoimmune Disord Vol 3:40.
Engelman R W, Tyler R D, Trang L Q, Liu W T, Good R A, Day N K. Clinicopathologic responses in cats with feline leukemia virus-associated leukemia-lymphoma treated with staphylococcal protein A. Am J Pathol. 1985 March; 118(3): 367-378.
Hoption Cann S A, van Netten J P, van Netten C. Dr William Coley and tumour regression: a place in history or in the future. Postgraduate Medical Journal 2003; 79:672-680.
Lo Russo G, Moro M, Sommariva M, Cancila V, Boeri M, Centonze G, Ferro S, Ganzinelli M, Gasparini P, Huber V, Milione M, Porcu L, Proto C, Pruneri G, Signorelli D, Sangaletti S, Sfondrini L, Storti C, Tassi E, Bardelli A, Marsoni S, Torri V, Tripodo C, Colombo M P, Anichini A, Rivoltini L, Balsari A, Sozzi G, Garassino M. Antibody-Fc/FcR interaction on macrophages as a mechanism for hyperprogressive disease in non-small cell lung cancer subsequent to PD-1/PD-L1 blockade. Clin Cancer Res 2019; 25:989-999.
Lu J, Mold C, Du Clos T W, Sun P D. Pentraxins and Fc Receptor-Mediated Immune Responses.
Frontiers in Immunology|www.frontiersin.org 2 Nov. 2018 |Volume 9 |Article 2607
Khagi Y, Goodman A M, Daniels G A, Patel S P, Sacco A G, Randall J M, Bazhenova L A, Kurzrock R. Hyper-Mutated Circulating Tumor DNA: Correlation with Response to Checkpoint Inhibitor-Based Immunotherapy. Clin Cancer Res. 2017 Oct. 1; 23(19): 5729-5736. doi:10.1158/1078-0432.CCR-17-1439.
Knorr D, Ravetch J V. Immunotherapy and hyperprogression: unwanted outcomes, unclear mechanism. Clin Cancer Res 2019; 25; 904-906.
Nilsson B, Moks T, Jansson B, Abrahmsen L, Elmblad A, Holmgren E, Henrichson C, Jones T. A, Uhlen M.
A synthetic IgG-binding domain based on staphylococcal protein A. Protein Engineering vol. 1 no. 2 pp. 107-113, 1987.
Ray, P. K., Bandyopadhyay, S., Dohadwala, M. et al. Antitumor activity with nontoxic doses of protein A. Cancer Immunology, Immunotherapy, October 1984, Volume 18, Issue 1, pp 29-34
Schuurhuis D H, Ioan-Facsinay A, Nagelkerken B, van Schip J J, Sedlik C, Melief C J, Verbeek J S, Ossendorp F. Antigen-Antibody Immune Complexes Empower Dendritic Cells to Efficiently Prime Specific CD8+ CTL Responses In Vivo. J Immunol 2002; 168:2240-2246.
Simpson T R, James P. Allison J P. Fc Gamma R. #Springer Science+Business Media New York 2017 J. L. Marshall (ed.), Cancer Therapeutic Targets, DOI 10.1007/978-1-4419-0717-2_140.
Sinclair N R. Letter: The role of the Fc portion in regulation of immune responses by antibody. Cell Immunol. 1975 September; 19(1):162-3.
Sinclair, N. R. StC., Lees, R. K., Elliott, E. V. (1968) Role of Fc fragment in the regulation of the primary immune response. Nature, 220, 1048 1049.)
Steele G Jr, Ankerst J, Sjögren H O, Vang J, Lannerstad O. Absorption of blocking activity from human tumor-bearer sera by Staphylococcus aureus, Cowan I. Int J Cancer. 1975 Feb. 15; 15(2):180-9.
Teige I, Mirtensson L, Frendéus B L. Targeting the Antibody Checkpoints to Enhance Cancer Immunotherapy-Focus on Fc RIIB. Frontiers in Immunology|www.frontiersin.org 1 Mar. 2019 Volume 10 Article 481.
Wen Y M, Mu L, Shi Y. Immunoregulatory functions of immune complexes in vaccine and therapy. EMBO Mol Med. 2016 Oct. 4; 8(10):1120-1133. doi: 10.15252/emmm.201606593. Print 2016 October
Wirth T C, Kühnel F. Neoantigen Targeting—Dawn of a New Era in Cancer Immunotherapy?Front Immunol. 2017; 8: 1848. Published online 2017 Dec. 19.
High COVID-19 virus replication rates, the creation of antigen-antibody immune complexes and indirect haemagglutination resulting in thrombosis. Kevin Roe. First published: 21 May 2020 doi.org/10.1111/tbed.13634
COVID-19 type III hypersensitivity reaction. Batool Mutar Mahdi Med Hypotheses. 2020 July; 140: 109763. Published online 2020 Apr. 23. doi: 10.1016/j.mehy.2020.109763 PMCID: PMC7177147 PMID: 32344307 www.ncbi.nlm.nih.gov/pmc/articles/PMC7177147/
Bad News Wrapped in Protein: Inside the Coronavirus Genome By Jonathan Corum and Carl ZimmerApr. 3, 2020
www.nytimes.com/interactive/2020/04/03/science/coronavirus-genome-bad-news-wrapped-in-protein.html?auth=login-email&login=email&utm_source=pocket-newtab
High-Dose IVIg Reversed Worsening COVID-19 in Case Reports. Mar. 25, 2020 KENNETH BENDER, PHARMD, M A www.contagionlive.com/news/highdose-ivig-reversed-worsening-covid19-in-case-reports
Therapeutic strategies in an outbreak scenario to treat the novel coronavirus originating in Wuhan, China. Robert L. Kruse. Furin cleavage site in the SARS-CoV-2 coronavirus glycoprotein. 13 Feb. 2020 www.virology.ws/2020/02/13/furin-cleavage-site-in-the-sars-cov-2-coronavirus-glycoprotein/
Autoimmun Rev. 2019 October; 18(10):102366. doi: 10.1016/j.autrev.2019.102366. Epub 2019 Aug. 9. Next-generation Fc receptor-targeting biologics for autoimmune diseases.
Zuercher AW1, Spirig R2, Baz Morelli A3, Rowe T3, Käsermann F4. Front Immunol. 2014; 5: 674. Published online 2015 Jan. 21. doi: 10.3389/fimmu.2014.00674
PMCID: PMC4301001 PMID: 25653650 Immunomodulation by IVIg and the Role of Fc-Gamma Receptors: Classic Mechanisms of Action after all? Sietse Q. Nagelkerkel and Taco W. Kuijpers1,2, www.ncbi.nlm.nih.gov/pmc/articles/PMC4301001/
Immunology. 2002 December; 107(4): 387-393. doi: 10.1046/j.1365-2567.2002.01545: Immunomodulatory action of intravenous immunoglobulin. W A C Sewell* and S Jolles†
N Engl J Med. 1982 May 27; 306(21):1254-8. Transient reversal of thrombocytopenia in idiopathic thrombocytopenic purpura by high-dose intravenous gamma globulin. Fehr J, Hofmann V, Kappeler U.
Tai, W., He, L., Zhang, X. et al. Characterization of the receptor-binding domain (RBD) of 2019 novel coronavirus: implication for development of RBD protein as a viral attachment inhibitor and vaccine. Cell Mol Immunol (2020). doi.org/10.1038/s41423-020-0400-4
J Immunol. 1966 December; 97(6):822-7. “Protein A” from S. aureus. I. Pseudo-immune reaction with human gamma-globulin. Forsgren A, Sjöquist J.
“Protein A” from Staphylococcus Aureus II. Arthus-like Reaction Produced in Rabbits by Interaction of Protein a and Human γ-Globulin G. T. Gustafson, J. Sjöquist and G. Stilenheim. J Immunol Jun. 1, 1967, 98 (6) 1178-1181;
A Medical Worker Describes Terrifying Lung Failure From COVID-19 Even in His Young Patients. www.propublica.org/article/a-medical-worker-describes--terrifying-lung-failure-from-covid19-even-in-his-young-patients
Clin Exp Immunol. 1983 April; 52(1): 164-172. Circulating immune complexes in patients with diabetes mellitus. C K Abrass, D Heber, and J Lieberman. Acta Diabetol Lat. 1982 April-June; 19(2):107-39. The role of circulating immune complexes in the pathogenesis of diabetes mellitus. Kilpatrick J M, Virella G.
Immune Mechanisms in Vascular Disease and Stroke Volume 2014|Article ID 504045 9 pagesdoi.org/10.1155/2014/504045
BioMed Research International/2014/Antibodies in the Pathogenesis of Hypertension www.hindawi.com/journals/bmri/2014/504045/mmunol Lett. 1986 August; 13(1-2):45-9. Characterization of circulating immune complexes in heart disease. Cristea A, Rus H, Niculescu F, Bedeleanu D, Vlaicu R.
Warning: Autoimmunity on the Rise in the U.S. Prevalence of antinuclear antibodies reached almost 16% of the population in 2011-2012. by Nancy Walsh, Senior Staff Writer, MedPage Today Apr. 8, 2020. www.medpagetoday.com/rheumatology/generalrheumatology/85842?xid=nl_mpt_DHE_2020-04-09&eun=g1350833d0r&utm_source=Sailthru&utm_medium=email&utm_campaign=Daily%20 Headlines%20Top%20Cat%20HeC%20%202020-04-09&utm_term=NL_Daily_DHE_dual-gmail-definition
Antinuclear antibodies: cause of disease or caused by disease? R. J. T. Smeenk Rheumatology, Volume 39, Issue 6, June 2000, Pages 581-584, doi.org/10.1093/rheumatology/39.6.581
Hypertension, Obesity Common in U.S. COVID-19 Hospitalizations—African Americans disproportionately affected www.medpagetoday.com/infectiousdisease/covid19/85855?xid=nl_popmed_2020-04-09&eun=g1350833d0r&utm_source=Sailthru&utm_medium=email&utm_campaign=CoronaBreak_040920&utm_term=NL_Daily_Breaking_News_Active.
J Int AIDS Soc. 2012 Jul. 10; 15(2):17393. doi: 10.7448/IAS.15.2.17393.
Abacavir alters the transcription of inflammatory cytokines in virologically suppressed, HIV-infected women. www.ncbi.nlm.nih.gov/pubmed/22789611
Effect of beta-blockers on circulating levels of inflammatory and anti-inflammatory cytokines in patients with dilated cardiomyopathy www.onlinejacc.org/content/37/2/412
Drummond, G. R., Vinh, A., Guzik, T. J. et al. Immune mechanisms of hypertension. Nat Rev Immunol 19, 517
Hypertension. 2018 April; 71(4): 719-728. Plasma cell depletion attenuates hypertension in an experimental model of autoimmune disease Erin B. Taylor, Michelle Barati, David W. Powell, Hannah R. Turbeville, Michael J. Ryan,
Anticoagulation Guidance Emerging for Severe COVID-19—Pragmatic choices dominate as guidelines are shaping up by Crystal Phend, Senior Editor, MedPage Today Apr. 8, 2020 www.medpagetoday.com/infectiousdisease/covid19/85865?utm_source=Sailthru&utm_medium=email&utm_campaign=Weekly%20Review %202020-04-12&utm_term=NL_DHE_Weekly_Active
What We Don't Know About the Coronavirus. By Clifford Marks and Trevor Pour Apr. 29, 2020. www.newyorker.com/science/medical-dispatch/what-we-dont-know-about-covid-19
Camussi G., Gavosto F. (1985) Immune Complexes and Thrombosis. In: Cajozzo A., Perricone R., Di Marco P., Palazzolo P. (eds) Advances in Hemostasis and Thrombosis. Ettore Majorana International Science Series, vol 20. Springer, Boston, Mass. link.springer.com/chapter/10.1007/978-1-4615-9424-6_16#citeas
Increasing the breadth and potency of response to the seasonal influenza virus vaccine by immune complex immunization
Jad Maamary, Taia T. Wang, Gene S. Tan, Peter Palese, and View ORCID ProfileJeffrey V. Ravetch PNAS Sep. 19, 2017 114 (38) 10172-10177; first published Sep. 5, 2017 doi.org/10.1073/pnas.1707950114
1. Contributed by Jeffrey V. Ravetch, Aug. 4, 2017 (sent for review May 16, 2017; reviewed by Anthony S. Fauci and Diane Mathis)
Amesh A. Adalja Are Immune Complexes Responsible for Severe Cases of 2009 H1N1 Influenza? Clinicians' Biosecurity News >2010 www.centerforhealthsecurity.org/cbn/2010/cbnreport_12172010.html
J. Am. Chem. Soc. 2006, 128, 35, 11463-11470 A Synthetic Mimic of Human Fc Receptors: Defined Chemical Modification of Cell Surfaces Enables Efficient Endocytic Uptake of Human Immunoglobulin-G Siwarutt Boonyarattanakalin, Scott E. Martin, Qi Sun, Blake R. Peterson doi.org/10.1021/ja062377w

What is claimed is:

1. A vaccine comprising:
an immunoglobulin binding factor (IBF) reagent;
At least one antigen or antigen immune complex (IC) reagent.

2. The vaccine of claim 1 wherein the IBF reagent is an Fc reagent, with collateral Fab reagent activity.

3. The vaccine of claim 1, wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized with FcR-Fab reagent binding units that can be further polymerized to form FcR-Fab reagent, an FcR fragmented to give a plurality of monovalent single (added because of difference in action MSPA) of FcR-Fab reagent binding units, an FcR comprising at least one FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof.

4. The vaccine of claim 1 wherein the IBF reagent is an FcR mimetic agent selected from the group consisting of complement reactive protein (CRP), complement Clq, microbial immunoglobulin binding factors (IBF), corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof.

5. The vaccine of claim 1 wherein the IBF is free from attachment to immune complex (IC).

6. The vaccine of claim 1 wherein the IBF is free from attachment to immune complex (IC) and is selected from the group consisting of a corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof.

7. The vaccine of claim 1 which has anti-viral activity.

8. The vaccine of claim 1 wherein the IC reagent is selected from the group consisting of synthetic, genetically engineered, monovalent, multivalent, isolated from a patient, a checkpoint inhibitor, and combinations thereof.

9. The vaccine of claim 1 for use in preventing and/or treating an immune complex disease or disorder in a patient.

10. The vaccine of claim 1 for use in preventing and/or treating a condition selected from the group consisting of cancer, abnormal immunity to antigen, abnormal FcR mediated immunity to antigen, and combinations thereof, in a patient.

11. The vaccine of claim 1 wherein the vaccine prevents and/or treats an immune complex disease or disorder in a patient.

12. The vaccine of claim 1 wherein the immune complex disease or disorder is selected from the group consisting of cancer, abnormal immunity to antigen, abnormal FcR mediated immunity to antigen, and combinations thereof.

13. The vaccine of claim 1 wherein the IBF reagent and IC reagent will not dissociate upon administration to a patient.

14. The vaccine of claim 1 wherein the binding between the IBF reagent and IC reagent is selected from the group consisting of van der Waals forces, hydrogen bonds, ionic bonds, hydrophobic interactions, and combinations thereof.

15. The vaccine of claim 1 wherein the IBF reagent and IC reagent are covalently bound.

16. The vaccine of claim 1 wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin.

17. The vaccine of claim 1 wherein IBF reagent is covalently bound to IC reagent by a peptide, by a non-peptide linker, or combinations thereof.

18. The vaccine of claim 1 wherein the vaccine treats and/or prevents a pathological infection.

19. The vaccine of claim 1 wherein the pathological infection is selected from the group consisting of bacterial infection, viral infection, fungal infection, parasitic infection, and combinations thereof.

20. A method of preventing and/or treating antigen antibody immune complex diseases and disorders in a patient, the method comprising the steps of:
selecting a patient in need of preventing and/or treating antigen antibody immune complex diseases and disorders;
administering to the patient a vaccine which comprises:
an immunoglobulin binding factor (IBF) reagent;
at least one immune complex (IC) reagent,
thereby preventing and/or treating antigen antibody immune complex diseases and disorders in the patient.

21. The method of claim 20 wherein the antigen antibody immune complex diseases and disorders involve cellular Fc receptor mediated pathological dysregulation or dysfunction of immune responses.

22. The method of claim 20 wherein the IBF reagent is an Fc reagent.

23. The method of claim 20, wherein the IBF reagent is an Fc reagent selected from the group consisting of a microbial Fc receptor mimic, a microbial Fc receptor mimic comprising both Fab and FcR activities, an Fc receptor mimic comprising both Fab and FcR activities, a checkpoint inhibitor, an Fc receptor isolated from a patient, a synthetic Fc Receptor, a genetically engineered Fc Receptor, a multivalent FcR, a leukocyte Fc receptor polypeptide modified to also bind antibody Fab, a microbial FcR polypeptide, an FcR isolated or polymerized with FcR-Fab reagent binding units that can be further polymerized to form FcR-Fab reagent, an FcR fragmented to give a plurality of monovalent single (added because of difference in action MSPA) of FcR-Fab reagent binding units, an FcR comprising at least one FcR-Fab reagent binding units, a microbial FcR mimic polypeptide, a bacterial FcR mimic polypeptide, a bacterial FcR mimic polypeptide with the albumin binding site removed, an IgG binding bacterial polypeptide, a Staphylococcus aureus protein A, a fragment of Staphylococcus protein A, a Staphylococcus aureus protein A with the albumin binding site removed, a streptococcal Protein G, a streptococcal Protein G with the albumin binding site removed, and combinations thereof.

24. The method of claim 20 wherein the IBF reagent is an FcR mimetic agent selected from the group consisting of complement reactive protein (CRP), complement Clq, microbial immunoglobulin binding factors (IBF), corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof.

25. The method of claim 20 wherein the IBF is free from attachment to immune complex (IC).

26. The method of claim 20 wherein the IBF is free from attachment to immune complex (IC) and is selected from the group consisting of a corona virus spike (S) protein, a fragment of a corona virus spike protein (S), a SARS-Cov2 spike protein (S), a fragment of a SARS-Cov2 spike protein (S), a fragment of a SARS-Cov2 spike protein (S) with the IBF FcR mimetic activity region, and combinations thereof, wherein the IBF can engage circulating IC to increase efficacious immunity, or diminish immunity, or immune complex hypersensitivity disease.

27. The method of claim 20, wherein the administration of a COVID-19 antigen(s) Spike (S)-protein with an IBF FcR mimetic region results in long lasting protective immunity with IBF interference.

28. The method of claim 20 wherein the vaccine has anti-viral activity.

29. The method of claim 20 wherein the IC reagent is selected from the group consisting of, synthetic, genetically engineered, monovalent, multivalent, isolated from a patient, a checkpoint inhibitor, and combinations thereof.

30. The method of claim 20 wherein the patient has an immune complex disease or disorder.

31. The method of claim 20 wherein the patient has a condition selected from the group consisting of cancer, abnormal immunity to antigen, abnormal FcR mediated immunity to antigen, and combinations thereof.

32. The method of claim 20 wherein the vaccine prevents and/or treats an immune complex disease or disorder.

33. The method of claim 20 wherein the immune complex disease or disorder is selected from the group consisting of cancer, abnormal immunity to antigen, abnormal FcR mediated immunity to antigen, and combinations thereof.

34. The method of claim 20 wherein the IBF reagent and IC reagent will not dissociate upon administration to a patient.

35. The method of claim 20 wherein the binding between the IBF reagent and IC reagent is selected from the group consisting of van der Waals forces, hydrogen bonds, ionic bonds, hydrophobic interactions, and combinations thereof.

36. The method of claim 20 wherein the IBF reagent and IC reagent are covalently bound.

37. The method of claim 20 wherein either IBF reagent or IC reagent is bound to avidin, streptavidin, or NeutrAvidin, or combinations thereof, and the other reagent is bound to biotin, and IBF reagent is bound to IC reagent by the interaction of biotin with avidin, streptavidin, or NeutrAvidin.

38. The method of claim 20 wherein IBF reagent is covalently bound to IC reagent by a peptide, by a non-peptide linker, or combinations thereof.

39. The method of claim 20 wherein the vaccine treats and/or prevents a pathological infection.

40. The method of claim 20 wherein the pathological infection is selected from the group consisting of bacterial infection, viral infection, fungal infection, parasitic infection, and combinations thereof.

41. The method of claim 20 wherein the vaccine is administered at a dose selected from the group consisting of about 0.1 ng to about 100 mg per day, or about 1 ng to about 10 mg per day, or about 10 ng to about 1 mg per day.

42. The method of claim 20 wherein the vaccine is administered to the patient on a regimen of, for example, one, two, three, four, five, six, or other doses per day.

43. The method of claim 20 wherein the vaccine is administered for example, for one day, two days, three days, four days, five days, six days, a week, two weeks, three weeks, four weeks, five weeks, six weeks, a month, two months, three months, four months, or more.

44. The method of claim 20 wherein the dose and treatment schedule of Fc reagent is flexible, individualized and varies with different phases of the immune complex disease, and from patient to patient, being raised or lowered according to alterations in the course of the disease or the development of undesirable effects and levels of biomarkers that predict efficacy or toxicity.

45. The method of claim 20 further comprising the step of the administering at least one therapeutic agent to the patient.

46. The method of claim 20 wherein the at least one additional therapeutic agent is administered prior to, concurrently with, subsequent to, or in combination with, the vaccine.
